(19) United States
(12) Patent Application Publication
(10) Pub. No.: US 2025/0205361 A1
(43) Pub. Date: Jun. 26, 2025

(54) TREATMENT REGIMENS FOR THERAPEUTIC EFFECTOR COMPONENTS THAT ACT VIA AN INTRACELLULAR MOLECULAR TARGET

(71) Applicant: Sapreme Technologies B.V., Bilthoven (NL)

(72) Inventors: Miriam Verena Bujny, Bilthoven (NL); Ingrid Jolanda de Vries-van Leeuwen, Bilthoven (NL); Lars Stefan Vorthoren, Bilthoven (NL)

(73) Assignee: Sapreme Technologies B.V., Bilthoven (NL)

(21) Appl. No.: 18/986,821

(22) Filed: Dec. 19, 2024

Related U.S. Application Data
(62) Division of application No. PCT/EP2024/064626, filed on May 28, 2024.

(30) Foreign Application Priority Data
Jun. 2, 2023 (NL) 2034978

(51) Int. Cl. A61K 48/00; A61K 9/00

(52) U.S. Cl. CPC A61K 48/0016; A61K 9/0019; A61K 48/0075; A61K 48/0083

(57) ABSTRACT

The present invention relates to the field of therapy. More in particular, the invention relates to therapeutic methods wherein an effector component is administered that requires cellular uptake to become effective and/or that exerts its effect via an intracellular (molecular) target, such as, but not limited to, nucleic acid or oligonucleotide therapeutics. The present invention provides modalities to extend the duration of effect of the effector component and/or extend the dosing interval of the effector component and/or reduce the dosing frequency of the effector component and/or to cause a (delayed) boost of the effect of the effector component.

### FIELD OF THE INVENTION

The present invention relates to the field of therapy. More in particular, the invention relates to therapeutic methods wherein an effector component is administered that requires cellular uptake to become effective and/or that exerts its effect via an intracellular (molecular) target, such as, but not limited to, nucleic acid or oligonucleotide therapeutics. The present invention provides modalities to extend the duration of effect of the effector component and/or extend the dosing interval of the effector component and/or reduce the dosing frequency of the effector component and/or to cause a (delayed) boost of the effect of the effector component.

### BACKGROUND OF THE INVENTION

The development of new drugs requires two major steps: the identification of a therapeutically relevant target and the development of a compound capable of modulating its function. Over the past century, drug development efforts were focused on targeting proteins with different types of compounds including small molecules and monoclonal antibodies. Many drugs have thus been developed for the treatment of a large spectrum of pathologies and, to date, protein targeting remains a privileged avenue in drug discovery. However, the development of a compound capable of inhibiting or activating the function of a protein requires the recognition of its complicated spatial conformation. Although some classes of proteins such as membrane receptors, enzymes, ion channels, or transport proteins can be therapeutically approached using conventional protein-targeting strategies, other targets like transcription factors, scaffold proteins, or structural proteins are much less druggable using traditional modalities.

An alternative to modulating the function of a protein is to modulate its expression and/or expression level, and this can be achieved by acting on its mRNA (messenger ribonucleic acid). Oligonucleotides are a class of single- or double-stranded small synthetic nucleic acid polymers (˜20-mer) that can be used to modulate gene expression.

Oligonucleotides act on gene expression via various mechanisms. They can target pre-mRNA, mRNA, or non-coding RNA to induce degradation, modulate splicing events, or interfere with protein translation. An example is a small interfering RNA (siRNA) for gene silencing, which results in inhibiting expression of the protein encoded by the silenced gene, in a process called RNA interference (RNAi). Oligonucleotides such as antisense oligonucleotides (AON, also referred to as ASO) can target an RNA transcript of a gene, wherein binding of the AON can result for example in restoration of a reading frame leading to e.g. translation into a partial functional protein. Transcriptional activation can also be achieved, using a specific class of oligonucleotide called small activating RNA (saRNA) through direct interaction with gene promoters. Since they execute their function by complete Watson-Crick base pairing with DNA or RNA, oligonucleotides can in theory target any gene of interest since only the right nucleotide sequence along the targeted DNA or RNA needs to be selected. This considerably expands the number of proteins that can be targeted through the modulation of their mRNA expression. In addition, non-coding RNA, including microRNA (miRNA or miR) or long non-coding RNA (lncRNA), which are emerging as potential therapeutic targets, can also be modulated by oligonucleotides. Furthermore, since the action of oligonucleotides requires high complementarity with the target sequence, oligonucleotides should, in principle, be much more specific than small molecule drugs.

The use of oligonucleotides as therapeutic agents was first proposed in the late 1970s. Extensive research programs aimed at chemically optimizing oligonucleotides have been undertaken since. Modifications of the phosphodiester bonds and of the sugar groups have been developed to improve oligonucleotide stability in plasma by increasing their resistance to nucleases and their affinity for serum proteins as well as their specificity for their target sequence. Formulations and conjugations with specific chemical groups were developed to overcome delivery limitations and tissue specificity. As a result of these efforts, antisense oligonucleotide (AON) therapies are now coming of age, with multiple approved drugs and dozens of late phase clinical trials ongoing.

Despite the progress that has been made over the past decades, efficient delivery of the oligonucleotide therapeutic to the target organ or tissue, penetration of the target tissue and, last but not least, the cellular uptake, still pose major challenges in the development of any new oligonucleotide therapeutic. Another major challenge stems from the fact that oligonucleotides only have transient effects and treatment has to be repeated, due to the turnover and clearance of oligonucleotides, target transcripts and proteins. The frequency depends on the target tissue, but also the dynamics of the target transcript and proteins.

Approaches to overcome these difficulties include, in particular, the conjugation of oligonucleotide therapeutics with targeting ligands combined with extensive chemical modifications. The development of liver-directed oligonucleotide therapeutics was transformed by direct conjugation of siRNA to a multivalent N-acetylgalactosamine (GalNAc) ligand combined with extensive chemical modifications to stabilize the siRNA allowed for selective targeting of hepatocytes in the liver through the asialoglycoprotein receptor (ASGPR). An early generation of GalNAc-conjugated siRNA, with only a few further modifications designated as standard template chemistry (STC), achieved clinical proof of concept but required a high and frequent dose regimen. Subsequent design improvements, which include the substitution of the two terminal phosphodiester linkages at the antisense 3′ and 5′ ends and the sense strand 5′ end with phosphorothioate linkages, led to the enhanced stabilization chemistry (ESC) design, with improved metabolic stability and potency enabling a reduction in total dose amount required and allowing for less frequent administration. GIVLAARI, the first GalNAc-conjugated siRNA with ESC design, received regulatory approval in 2019. It is dosed at 2.5 mg/kg monthly by subcutaneous injection. Continued refinement of the chemical modification pattern has led to the development of advanced ESC designs with increased metabolic stability (Foster et al., Advanced siRNA Designs Further Improve In Vivo Performance of GalNAc-siRNA Conjugates, Molecular Therapy Vol. 26 No 3, pp. 708-717, March 2018), and inclusion of seed destabilizing modifications, like glycol nucleic acid, provides improved specificity, designated as ESC+ design or AdvESC. These advances have resulted in prolonged duration of target protein reduction in nonclinical and clinical studies. Vutrisiran, an advanced ESC GalNAc-conjugated siRNA that has recently received regulatory approval, for treatments involving subcutaneous (s.c.) administration once every 3 months, demonstrated sustained pharmacodynamic (PD) effect lasting up to 10 months after a single 25-mg s.c. dose.

Currently approved oligonucleotide therapeutics mainly target rare diseases and therapies are often very expensive. Moreover, most oligonucleotide therapeutics require treatment in a hospital (e.g. because they are administered intrathecally and/or intravenously), and thus give rise to significant additional costs and burden to health care systems that are already under (growing) strain. Hence, it would be highly desirable for new modalities to become available that can extend the duration of effect of oligonucleotide therapeutics, which, ideally, have general/wide applicability, would not require treatment in the hospital setting and offer the prospect of becoming (clinically) available at reasonable cost.

It is an objective of the present invention to provide such new modalities for improving oligonucleotide based therapies, in particular for extending the duration of effect of an oligonucleotide therapeutic and/or extending the dosing interval of an oligonucleotide therapeutic and/or reducing of the dosing frequency of an oligonucleotide therapeutic.

### SUMMARY OF THE INVENTION

The present invention resides in the finding that nucleic acid or oligonucleotide therapeutics, as well as other therapeutic effector molecules that require cellular uptake to become effective, such as a toxin, an enzyme, or a small molecule therapeutic (hereinafter collectively referred to as an ‘effector component’), persist for very long periods of time in cellular endosomes and that such effector components can be released from these endosomes by the action of a saponin component, long after the effector component was administered, in amounts sufficient to cause a therapeutically meaningful boost in the effect of said effector component (without actually administering a further dose of it). The durability of this response has proven to be remarkably high. More in particular, as shown in the experimental section of this document (‘Examples’), this principle has been demonstrated in an in vivo experiment, by treating mice with GN3-siTTR (an siRNA with advanced ESC design) followed by the administration of GN3-SO1861 (a trimeric GalNAc—saponin conjugate, also referred to as a trivalent GalNAc—saponin conjugate) at different points in time. It has been shown in these experiments that GN3-siTTR depots are accessible with GN3-saponin to release the siRNA, with a maximum response (suppression of TTR protein expression) higher than achieved with GN3-siTTR treatment alone, even when the interval between GN3-siTTR and GN3-saponin administration was 28 days. The experiment showed that GN3-saponin administration did not cause endosomal disruption or tolerability issues. Multiple in vitro experiments have shown that comparable effects can be attained with other oligonucleotide therapeutics as well as other (non-oligonucleotide) effector components. Taken together, the data currently available shows that, surprisingly, saponins can increase the potency of a preloaded effector component in a timed and inducible manner, after a resting period. The effects observed thus support the use of a saponin component to attain a meaningful extension of the duration of effect and/or a meaningful extension of the dosing interval and/or a meaningful reduction of the dosing frequency of an effector component and/or a meaningful (delayed) boost in the effect of the effector component, such as, in particular, an oligonucleotide therapeutic. The experiments underlying the present invention included oligonucleotide therapeutics with advanced ESC designs. Even with these oligonucleotides, which already possess significantly increased metabolic stability, substantial further extension of the duration of effect has been demonstrated. From the literature, it is known that pharmacokinetic and/or pharmacodynamic effects of oligonucleotide therapeutics observed in animals/animal models, can reliably be extrapolated/translated to other species, including humans. McDougal et al. (The Nonclinical Disposition and Pharmacokinetic/Pharmacodynamic Properties of N-Acetylgalactosamine-Conjugated Small Interfering RNA Are Highly Predictable and Build Confidence in Translation to Human; Drug Metab Dispos 50:781-797, June 2022), for instance report the results of studies demonstrating that the PK/PD and ADME properties of GalNAc-conjugated siRNAs are highly conserved across species. Based on their results McDougal et al. state that results obtained in animals can accurately be scaled to human, allowing to identify efficacious and safe clinical dosing regimens in the absence of human liver PK profiles.

According to the inventor's best knowledge, the literature concerning the use of saponins for endosomal escape enhancement has never alluded to the administration regimens and/or effects that are the subject of the present invention. At present, most of the literature concerning saponins as endosomal escape enhancing moieties teaches or hints at the concurrent use of the saponin and the effector component. Table D, incorporated in the detailed description below, recites a number of (prior art) examples wherein combinations of a saponin component and an effector component have been tested for use in the treatment or prophylaxis of a variety of diseases. These prior art teachings do not in any way disclose or hint at the general concept underlying the present invention, according to which the saponin component is used/administered with the purpose of extending the duration of effect, extending the dosing interval, reducing the dosing frequency and/or creating a (delayed) boost in the effect of the effector component. More in particular, in these examples the effector component and the saponin component are invariably administered together in the in vitro and in vivo models for assessing the stimulatory effect of the saponin on the activity and efficacy of the effector molecule.

To the extent that sequential treatment has been disclosed in the art, it involved priming with a saponin, followed by the administration of the effector component. For instance, Mitdank et al. (Suicide nanoplasmids coding for ribosome-inactivating proteins; European Journal of Pharmaceutical Sciences 170 (2022) 106107), describe an in vivo study (in mice) wherein AG1856 was administered (subcutaneously) 1 hour prior to the (intravenous) administration of ‘suicide nanoplexes’. Bachran et al. (The distribution of saponins in vivo affects their synergy with chimeric toxins against tumours expressing human epidermal growth factor receptors in mice; British Journal of Pharmacology (2010) 159 345-352), describe the results of an in vivo study (in tumour-bearing mice) relying on the sequential administration of Saponinum album followed by the administration of a chimeric toxin against the epidermal growth factor receptor, ErbB1. Bachran et al. report that there was high antitumour efficacy (66% inhibition of tumour growth) when the toxin was administered after 60 minutes, following pre-treatment with the saponin, but no significant inhibition when it was administered already after 10 minutes following pre-treatment. Panjideh et al. (Improved Therapy of B-Cell Non-Hodgkin Lymphoma by Obinutuzumab-Dianthin Conjugates in Combination with the Endosomal Escape Enhancer SO1861; Toxins (2022) 14, 478, doi.org/10.3390/toxins14070478) describe the results of an in vivo study (in mice), where mice in the treatment group received the saponin SO1861 subcutaneously, followed by the administration (intraperitoneally) of obinutuzumab-dianthin, one hour later.

The extension of the duration of effect, extension of the dosing interval, reduction in the dosing frequency and/or the delayed boost in the effect are particularly pronounced and advantageous in case the effector component is a nucleic acid or oligonucleotide therapeutic. However, as will be apparent to those skilled in the art, based on the present teachings, similar advantageous effects can be attained in case of other types of effector components (that require cellular uptake to become effective), such as toxins, enzymes, small molecule therapeutics, etc., and such embodiments are thus also encompassed by the present invention.

Hence, generally stated, the invention concerns a therapeutic method of treatment of a subject suffering from a disease or condition; said therapeutic method of treatment comprising:
i) the administration, to said subject, preferably the repeated administration, of an effector component that is capable of modulating an intracellular process involved in the disease or condition or of an intracellular process that can aid in the ailment of said disease or condition, including the relief of a symptom of the disease or condition; and
ii) the administration, to said subject, of a saponin component;
wherein the administration of the saponin component results in an extension of the effect of the effector component and/or in an extension of the dosing interval of the effector component and/or in a reduction of the dosing frequency of the effector component and/or in a (delayed) boost of the effect of the effector component.

A first particular aspect of the invention concerns a therapeutic method of treatment of a subject suffering from a disease or condition related to a defect in (the expression of) a gene and/or a disease or condition that is treatable by modulating the expression and/or expression level of a gene; said therapeutic method of treatment comprising:
i) the administration, to said subject, preferably the repeated administration, of a nucleic acid or oligonucleotide therapeutic that is capable of modulating the expression and/or expression level of said gene; and
ii) the administration, to said subject, of a saponin component;
wherein the administration of the saponin component results in an extension of the duration of effect of the nucleic acid or oligonucleotide therapeutic and/or in an extension of the dosing interval of the nucleic acid or oligonucleotide therapeutic and/or in a reduction of the dosing frequency of the nucleic acid or oligonucleotide therapeutic and/or in a (delayed) boost in the effect of the nucleic acid or oligonucleotide therapeutic.

In a second aspect, the present invention concerns a saponin component for use in a therapeutic method of treating a subject suffering from a disease or condition related to a defect in (the expression of) a gene and/or a disease or condition that is treatable by modulating the expression and/or expression level of a gene; said therapeutic method comprising:
i) the administration, to said subject, preferably the repeated administration, of a nucleic acid or oligonucleotide therapeutic that is capable of modulating the expression and/or expression level of said gene; and
ii) the administration, to said subject, of the saponin component;
wherein the administration of the saponin component results in an extension of the duration of effect of the nucleic acid or oligonucleotide therapeutic and/or in an extension of the dosing interval of the nucleic acid or oligonucleotide therapeutic and/or in a reduction of the dosing frequency of the nucleic acid or oligonucleotide therapeutic and/or in a (delayed) boost in the effect of the nucleic acid or oligonucleotide therapeutic. A further aspect, concerns a nucleic acid or oligonucleotide therapeutic that is capable of modulating the expression and/or expression level of a gene for use in a therapeutic method of treating a subject suffering from a disease or condition related to a defect in (the expression of) said gene and/or a disease or condition that is treatable by modulating the expression and/or expression level of said gene; said therapeutic method comprising:
i) the administration, to said subject, preferably the repeated administration, of said nucleic acid or oligonucleotide therapeutic; and
ii) the administration, to said subject, of a saponin component;
wherein the administration of the saponin component results in an extension of the duration of effect of the nucleic acid or oligonucleotide therapeutic and/or in an extension of the dosing interval of the nucleic acid or oligonucleotide therapeutic and/or in a reduction of the dosing frequency of the nucleic acid or oligonucleotide therapeutic and/or in a (delayed) boost in the effect of the nucleic acid or oligonucleotide therapeutic.

A further aspect of the invention concerns the use of a saponin component in the manufacture of a medicament for use in a therapeutic method of treatment of a subject suffering from a disease or condition related to a defect in (the expression of) a gene and/or a disease or condition that is treatable by modulating the expression and/or expression level of a gene; said therapeutic method of treatment comprising:
i) the administration, to said subject, preferably the repeated administration, of a nucleic acid or oligonucleotide therapeutic that is capable of modulating the expression and/or expression level of said gene; and
ii) the administration, to said subject, of the saponin component;
wherein the administration of the saponin component results in an extension of the duration of effect of the nucleic acid or oligonucleotide therapeutic and/or in an extension of the dosing interval of the nucleic acid or oligonucleotide therapeutic and/or in a reduction of the dosing frequency of the nucleic acid or oligonucleotide therapeutic and/or in a (delayed) boost in the effect of the nucleic acid or oligonucleotide therapeutic. A further aspect concerns the use of a nucleic acid or oligonucleotide therapeutic that is capable of modulating the expression and/or expression level of a gene, in the manufacture of a medicament for use in a therapeutic method of treatment of a subject suffering from a disease or condition related to a defect in (the expression of) said gene and/or a disease or condition that is treatable by modulating the expression and/or expression level of said gene; said therapeutic method of treatment comprising:
i) the administration, to said subject, preferably the repeated administration, of said nucleic acid or oligonucleotide therapeutic; and
ii) the administration, to said subject, of a saponin component;
wherein the administration of the saponin component results in an extension of the duration of effect of the nucleic acid or oligonucleotide therapeutic and/or in an extension of the dosing interval of the nucleic acid or oligonucleotide therapeutic and/or in a reduction of the dosing frequency of the nucleic acid or oligonucleotide therapeutic and/or in a (delayed) boost in the effect of the nucleic acid or oligonucleotide therapeutic.

Yet, a further aspect of the invention concerns a pharmaceutical combination comprising a saponin component and a nucleic acid or oligonucleotide therapeutic that is capable of modulating the expression and/or expression level of a gene, for use in a therapeutic method of treatment of a disease or condition related to a defect in (the expression of) said gene and/or a disease or condition that is treatable by modulating the expression and/or expression level of said gene, said therapeutic method comprising:
i) the administration, to said subject, preferably the repeated administration, of said nucleic acid or oligonucleotide therapeutic; and
ii) the administration, to said subject, of the saponin component;
wherein the administration of the saponin component results in an extension of the duration of effect of the nucleic acid or oligonucleotide therapeutic and/or in an extension of the dosing interval of the nucleic acid or oligonucleotide therapeutic and/or in a reduction of the dosing frequency of the nucleic acid or oligonucleotide therapeutic and/or in a (delayed) boost in the effect of the nucleic acid or oligonucleotide therapeutic.

Yet, a further aspect of the invention concerns a pharmaceutical kit comprising a package comprising a) one or more dosage units comprising a saponin component, and b) printed instructions to use the dosage units comprised in the kit in a therapeutic method of treatment of a disease or condition related to a defect in (the expression of) a gene and/or a disease or condition that is treatable by modulating the expression and/or expression level of a gene, said method of treatment comprising:
i) the administration, preferably the repeated administration, of a nucleic acid or oligonucleotide therapeutic that is capable of modulating the expression and/or expression level of said gene,
ii) the administration, to said subject, of the saponin component;
wherein the administration of the saponin component results in an extension of the duration of effect of the nucleic acid or oligonucleotide therapeutic and/or in an extension of the dosing interval of the nucleic acid or oligonucleotide therapeutic and/or in a reduction of the dosing frequency of the nucleic acid or oligonucleotide therapeutic and/or in a (delayed) boost in the effect of the nucleic acid or oligonucleotide therapeutic.

Yet, a further aspect of the invention concerns a pharmaceutical kit comprising a package comprising a) one or more dosage units comprising a saponin component; b) one or more dosage units comprising a nucleic acid or oligonucleotide therapeutic that is capable of modulating the expression and/or expression level of a gene; and c) printed instructions to use the dosage units comprised in the kit in a therapeutic method for the treatment of a disease or condition related to a defect in (the expression of) said gene and/or a disease or condition that is treatable by modulating the expression and/or expression level of said gene; said method comprising:
i) the administration, to said subject, preferably the repeated administration, of said nucleic acid or oligonucleotide therapeutic; and
ii) the administration, to said subject, of the saponin component;
wherein the administration of the saponin component results in an extension of the duration of effect of the nucleic acid or oligonucleotide therapeutic and/or in an extension of the dosing interval of the nucleic acid or oligonucleotide therapeutic and/or in a reduction of the dosing frequency of the nucleic acid or oligonucleotide therapeutic and/or in a (delayed) boost in the effect of the nucleic acid or oligonucleotide therapeutic.

It will be apparent to those skilled in the art, based on the present disclosure, that the main aspects of the invention, as defined here above, are based on the same innovative concepts. The innovative concepts presented herein will be described with respect to particular embodiments. Unless stated otherwise and/or unless something else is apparent from the context, the particular embodiments described herein below apply, indiscriminately, to each and every one of the aspects of the invention as defined herein above. Particular embodiments described herein should be regarded as descriptive and not limiting beyond of what is described in the claims. The embodiments as described herein can operate in combination and cooperation, unless specified otherwise.

### Definitions

As used herein, the term “saponin component” has its regular scientific meaning and here refers to a component comprising a saponin moiety or consisting of a saponin molecule.

The term “saponin” has its regular scientific meaning and here refers to a group of amphipathic glycosides which comprise one or more hydrophilic glycone moieties combined with a lipophilic aglycone core which is a sapogenin. The saponin may be naturally occurring or synthetic (i.e. non-naturally occurring). The term “saponin” includes naturally-occurring saponins, derivatives of naturally-occurring saponins as well as saponins synthesized de novo through chemical and/or biotechnological synthesis routes.

The term “aglycone core structure” has its regular scientific meaning and here refers to the aglycone core of a saponin without the one or two carbohydrate antenna or saccharide chains (glycans) bound thereto. For example, quillaic acid is the aglycone glycoside core structure for SO1861, QS-7, and QS-21.

The term “saccharide chain” or “carbohydrate chain” has its regular scientific meaning and here refers to any of a glycan, a carbohydrate antenna, a single saccharide moiety (monosaccharide) or a chain comprising multiple saccharide moieties (oligosaccharide, polysaccharide). The saccharide chain can consist of only saccharide moieties or may also comprise further moieties such as any one of 4E-Methoxycinnamic acid, 4Z-Methoxycinnamic acid, and 5-O-[5-O-Ara/Api-3,5-dihydroxy-6-methyl-octanoyl]-3,5-dihydroxy-6-methyl-octanoic acid), such as for example present in QS-21.

The term “Api/Xyl-” or “Api- or Xyl-” in the context of the name of a saccharide chain has its regular scientific meaning and here refers to the saccharide chain either comprising an apiose (Api) moiety, or comprising a xylose (Xyl) moiety.

The term “conjugate” has its regular scientific meaning and here refers to at least a first molecule that is covalently bound to at least a second molecule, therewith forming a covalently coupled assembly comprising or consisting of the first molecule and the second molecule. Typical conjugates are (GalNAc)3-siRNA (or GN3-siRNA), an ADC, an AOC, and SO1861-EMCH (EMCH linked to the aldehyde group of the aglycone glycoside core structure of the saponin, according to formula (I) (see below)). As used herein, the term “conjugate” is thus to be construed as a combination of two or more different molecules that have been and are covalently bound. For example, different molecules forming a conjugate as disclosed herein may include one or more saponins or saponin molecules with one or more ligands that bind to an endocytic receptor present on a surface of a muscle cell, a hepatocyte, a tumor cell, preferably wherein the ligand is one or more GalNAc moieties, an antibody or a binding fragment thereof, such as an IgG, a monoclonal antibody (mAb), a single domain antibody such as a VHH domain or another nanobody type, a bivalent nanobody molecule comprising two single domain antibodies, etc. In some aspects, the disclosed herein conjugates may be made by covalently linking different molecules via one or more intermediate molecules such as linkers, such as for example via linking to a central or further linker. In a conjugate, not all of the two or more, such as three, different molecules need to be directly covalently bound to each other. Different molecules in the conjugate may also be covalently bound by being both covalently bound to the same intermediate molecule such as a linker or each by being covalently bound to an intermediate molecule such as a further linker or a central linker wherein these two intermediate molecules such as two (different) linkers, are covalently bound to each other. According to this definition even more intermediate molecules, such as linkers, may be present between the two different molecules in the conjugate as long as there is a chain of covalently bound atoms in between.

The term “Saponinum album” has its normal meaning and here refers to a mixture of saponins produced by Merck KGaA (Darmstadt, Germany) containing saponins from Gypsophila paniculata and Gypsophila arostii, containing SA1657 and mainly SA1641.

The term “Quillaja saponin” has its normal meaning and here refers to the saponin fraction of Quillaja saponaria and thus the source for all other QS saponins, mainly containing QS-18 and QS-21.

“QS-21” or “QS21” has its regular scientific meaning and here refers to a mixture of QS-21 A-apio (˜63%), QS-21 A-xylo (˜32%), QS-21 B-apio (˜3.3%), and QS-21 B-xylo (˜1.7%).

Similarly, “QS-21A” has its regular scientific meaning and here refers to a mixture of QS-21 A-apio (˜65%) and QS-21 A-xylo (˜35%).

Similarly, “QS-21 B” has its regular scientific meaning and here refers to a mixture of QS-21 B-apio (˜65%) and QS-21 B-xylo (˜35%).

The term “Quil-A” refers to a commercially available semi-purified extract from Quillaja saponaria and contains variable quantities of more than 50 distinct saponins, many of which incorporate the triterpene-trisaccharide substructure Gal-(1→2)-[Xyl-(1→3)]-GIcA- at the C-3beta-OH group found in QS-7, QS-17, QS-18, and QS-21. The saponins found in Quil-A are listed in van Setten (1995), Table 2 [Dirk C. van Setten, Gerrit van de Werken, Gijsbert Zomer and Gideon F. A. Kersten, Glycosyl Compositions and Structural Characteristics of the Potential Immuno-adjuvant Active Saponins in the Quillaja saponaria Molina Extract Quil A, RAPID COMMUNICATIONS IN MASS SPECTROMETRY, VOL. 9,660-666 (1995)]. Quil-A and also Quillaja saponin are fractions of saponins from Quillaja saponaria and both contain a large variety of different saponins with largely overlapping content. The two fractions differ in their specific composition as the two fractions are gained by different purification procedures.

The term “QS1861” and the term “QS1862” refer to QS-7 and QS-7 api. QS1861 has a molecular mass of 1861 Dalton, QS1862 has a molecular mass of 1862 Dalton. QS1862 is described in Fleck et al. (2019) in Table 1, row no. 28 [Juliane Deise Fleck, Andresa Heemann Betti, Francini Pereira da Silva, Eduardo Artur Troian, Cristina Olivaro, Fernando Ferreira and Simone Gasparin Verza, Saponins from Quillaja saponaria and Quillaja brasiliensis: Particular Chemical Characteristics and Biological Activities, Molecules 2019, 24, 171; doi:10.3390/molecules24010171]. The described structure is the api-variant QS1862 of QS-7. The molecular mass is 1862 Dalton as this mass is the formal mass including proton at the glucuronic acid. At neutral pH, the molecule is deprotonated. When measuring in mass spectrometry in negative ion mode, the measured mass is 1861 Dalton.

The terms “SO1861” and “SO1862” refer to the same saponin of Saponaria officinalis, though in deprotonated form or api form, respectively. The molecular mass is 1862 Dalton as this mass is the formal mass including a proton at the glucuronic acid. At neutral pH, the molecule is deprotonated. When measuring the mass using mass spectrometry in negative ion mode, the measured mass is 1861 Dalton.

As used herein, the term “effector component” has its regular scientific meaning and here refers to a component comprising or consisting of an effector molecule or moiety. An example of an effector component according to the invention is a nucleic acid therapeutic or oligonucleotide therapeutic, in which the nucleic acid or oligonucleotide is the effector molecule or the effector moiety.

The term “effector molecule”, or “effector moiety” when referring to the effector molecule as part of e.g. a covalent conjugate such as an effector component comprising a ligand for binding to an endocytic cell-surface receptor and comprising e.g. a nucleic acid, has its regular scientific meaning and here refers to a molecule that can selectively bind to for example any one or more of the target molecules: a protein, a peptide, a carbohydrate, a saccharide such as a glycan, a (phospho)lipid, a nucleic acid such as DNA, RNA, an enzyme, and that regulates the biological activity of such one or more target molecule(s). In the effector molecule according to the invention the effector moiety for example exerts its effect in the cytosol (cytoplasm) and/or in the cell nucleus, and/or is delivered intracellularly in the endosome and/or lysosome and/or is active after exiting or escaping the endosomal-lysosomal pathway (therewith entering the cytoplasm). The effector molecule is for example a molecule selected from any one or more of a small molecule such as a drug molecule, a toxin such as a protein toxin, a nucleic acid or polynucleotide such as a BNA, an ASO, a PMO, a xeno nucleic acid or an siRNA, an enzyme, a peptide, a protein, or an active fragment or active domain thereof, or any combination thereof. Thus, for example, an effector molecule or an effector moiety is a molecule or moiety selected from any one or more of a small molecule such as a drug molecule, a toxin such as a protein toxin, a nucleic acid or polynucleotide such as a BNA, an ASO, a PMO, a xeno nucleic acid or an siRNA, an enzyme, a peptide, a protein, or any combination thereof, that can selectively bind to any one or more of the target molecules: a protein, a peptide, a carbohydrate, a saccharide such as a glycan, a (phospho)lipid, a nucleic acid such as DNA, RNA, an enzyme, and that upon binding to the target molecule regulates the biological activity of such one or more target molecule(s). For example, an effector moiety is a toxin or an active toxic fragment thereof or an active toxic derivative or an active toxic domain thereof. Typically, an effector molecule can exert a biological effect inside a cell such as a mammalian cell such as a human cell, such as in the cytosol of said cell or in the nucleus of said cell. An effector molecule or moiety of the invention is thus any substance that affects the metabolism of a cell by interaction with an intracellular effector molecule target, wherein this effector molecule target is any molecule or structure inside cells excluding the lumen of compartments and vesicles of the endocytic and recycling pathway but including the membranes of these compartments and vesicles. Said structures inside cells thus include the nucleus, mitochondria, chloroplasts, endoplasmic reticulum, Golgi apparatus, other transport vesicles, the inner part of the plasma membrane and the cytosol. Typical effector molecules are thus drug molecules, an enzyme, a nucleic acid such as plasmid DNA or an ASO or an siRNA or a PMO, toxins such as toxins comprised by antibody-drug conjugates (ADCs), polynucleotides such as siRNA, BNA, nucleic acids comprised by an antibody-polynucleotide conjugate (AOC). For example, an effector molecule/moiety is a molecule which can act as a ligand that can increase or decrease (intracellular) enzyme activity, gene expression (e.g. gene silencing), or cell signalling. Typically, an effector moiety comprised by the conjugate exerts its therapeutic (for example toxic, enzymatic, inhibitory, gene silencing, etc.) effect in the cytosol and/or in the cell nucleus. Typically, the effector moiety is delivered intracellularly in the endosome and/or in the lysosome, and typically the effector moiety is active after exiting or escaping the endosomal-lysosomal pathway. Within the saponin component according to the invention saponin is not considered an effector molecule nor an effector moiety in the saponin component according to the invention. Thus, in the saponin components comprising a saponin, the saponin is not an effector moiety, and in the effector components comprising an effector moiety, the effector moiety is a different molecule than a conjugated saponin. In the context of the saponin component of the invention, the term saponin refers to those saponins which exert an endosomal/lysosomal escape enhancing activity, when present in the endosome and/or lysosome of a mammalian cell such as a human cell, towards an effector moiety comprised by the effector component of the invention and present in said endosome/lysosome together with the saponin.

As used herein, the terms “nucleic acid” and “oligonucleotide” are synonymous to one another and are to be construed as encompassing any polymeric molecule made of units, wherein a unit comprises a nucleobase (or simply “base” e.g. being a canonical nucleobase like adenine (A), cytosine (C), guanine (G), thymine (T), or uracil (U), or any known non-canonical, modified, or synthetic nucleobase like 5-methylcytosine, 5-hydroxymethylcytosine, xanthine, hypoxanthine, 7-methylguanine; 5,6-dihydrouracil etc.) or a functional equivalent thereof, which renders said polymeric molecule capable of engaging in hydrogen bond-based nucleobase pairing (such as Watson-Crick base pairing) under appropriate hybridisation conditions with naturally-occurring nucleic acids such as deoxyribonucleic acid (DNA) or ribonucleic acid (RNA), which naturally-occurring nucleic acids are to be understood being polymeric molecules made of units being nucleotides.

Hence, from a chemistry perspective, the term nucleic acid and the term oligonucleotide under the present definition can be construed as encompassing polymeric molecules that chemically are DNA or RNA, as well as polymeric molecules that are nucleic acid analogues, also known as xeno nucleic acids (XNA) or artificial nucleic acids, which are polymeric molecules wherein one or more (or all) of the units are modified nucleotides or are functional equivalents of nucleotides. Nucleic acid analogues are well known in the art and due to various properties, such as improved specificity and/or affinity, higher binding strength to their target and/or increased stability in vivo, they are extensively used in research and medicine. Typical examples of nucleic acid analogues include but are not limited to locked nucleic acid (LNA) (that is also known as bridged nucleic acid (BNA)), phosphorodiamidate morpholino oligomer (PMO also known as Morpholino), peptide nucleic acid (PNA), glycol nucleic acid (GNA), threose nucleic acid (TNA), hexitol nucleic acid (HNA), 2′-deoxy-2′-fluoroarabinonucleic acid (FANA or FNA), 2′-deoxy-2′-fluororibonucleic acid (2′-F RNA or FRNA); altritol nucleic acids (ANA), cyclohexene nucleic acids (CeNA) etc.

In accordance with the canon, length of a nucleic acid (oligonucleotide) is expressed herein the number of units from which a single strand of a nucleic acid is build. Because each unit corresponds to exactly one nucleobase capable of engaging in one base pairing event, the length is frequently expressed in so called “base pairs” or “bp” regardless of whether the nucleic acid in question is a single stranded (ss) or double stranded (ds) nucleic acid. In naturally-occurring nucleic acids 1 bp corresponds to 1 nucleotide, abbreviated to 1 nt. For example, a single stranded nucleic acid made of 1000 nucleotides (or a double stranded nucleic acid made of two complementary strands each of which is made of 1000 nucleotides) is described as having a length of 1000 base pairs or 1000 bp, which length can also be expressed as 1000 nt or as 1 kilobase that is abbreviated to 1 kb. 2 kilobases or 2 kb are equal to the length of 2000 base pair which equates 2000 nucleotides of a single stranded RNA or DNA. To avoid confusion however, in view of the fact the nucleic acids as defined herein may comprise or consist of units not only chemically being nucleotides but also being functional equivalents thereof, the length of nucleic acids will preferentially be expressed herein in “bp” or “kb” rather than in the equally common in the art denotation “nt”.

In advantageous embodiments as disclosed herein, the nucleic acids (or oligonucleotides) are no longer than 1kb, preferably no longer than 500 bp, most preferably no longer than 250 bp.

In particularly advantageous embodiments, the nucleic acid is an oligonucleotide (or simply an oligo) defined as nucleic acid being no longer than 150 bp, i.e. in accordance with the above provided definition, being any polymeric molecule made of no more than 150 units, wherein each unit comprises a nucleobase or a functional equivalent thereof, which renders said oligonucleotide capable of engaging in hydrogen bond-based nucleobase pairing under appropriate hybridisation conditions with DNA or RNA. Within the ambit of said definition, it will immediately be appreciated that the disclosed herein oligonucleotides can comprise or consist of units not only being nucleotides but also being synthetic equivalents thereof. In other words, from a chemistry perspective, as used herein the term oligonucleotide will be construed as possibly comprising or consisting of RNA, DNA, or a nucleic acid analogue such as but not limited to LNA (BNA), PMO (Morpholino), PNA, GNA, TNA, HNA, FANA, FRNA, ANA, CeNA and/or the like.

The term “proteinaceous” has its regular scientific meaning and here refers to a molecule that is protein-like, meaning that the molecule possesses, to some degree, the physicochemical properties characteristic of a protein, is of protein, relating to protein, containing protein, pertaining to protein, consisting of protein, resembling protein, or being a protein. The term “proteinaceous” as used in for example ‘proteinaceous molecule’ refers to the presence of at least a part of the molecule that resembles or is a protein, wherein ‘protein’ is to be understood to include a chain of amino-acid residues at least two residues long, thus including a peptide, a polypeptide and a protein and an assembly of proteins or protein domains. In the proteinaceous molecule, the at least two amino-acid residues are for example linked via (an) amide bond(s), such as (a) peptide bond(s). In the proteinaceous molecule, the amino-acid residues are natural amino-acid residues and/or artificial amino-acid residues such as modified natural amino-acid residues. In a preferred embodiment, a proteinaceous molecule is a molecule comprising at least two amino-acid residues, preferably between two and about 2.000 amino-acid residues. In one embodiment, a proteinaceous molecule is a molecule comprising from 2 to 20 (typical for a peptide) amino acids. In one embodiment, a proteinaceous molecule is a molecule comprising from 21 to 1.000 (typical for a polypeptide, a protein, a protein domain, such as an antibody, a Fab, an scFv, a ligand for a receptor such as EGF) amino acids. Preferably, the amino-acid residues are (typically) linked via (a) peptide bond(s). According to the invention, said amino-acid residues are or comprise (modified) (non-)natural amino acid residues.

As used herein, the term “antibody or a binding fragment thereof or a binding domain thereof” refers to a polypeptide that includes at least one immunoglobulin variable domain or at least one antigenic determinant, e.g., paratope that specifically binds to an antigen. In some embodiments, an antibody is a full-length antibody. In some embodiments, an antibody is a chimeric antibody. In some embodiments, an antibody is a humanized antibody. However, in some embodiments, an antibody is a Fab fragment, a F(ab′) fragment, a F(ab′)2 fragment, a Fv fragment or a scFv fragment. In some embodiments, an antibody is a nanobody derived from a camelid antibody or a nanobody derived from a shark antibody. In some embodiments, an antibody is a diabody. In some embodiments, an antibody comprises a framework having a human germline sequence. In another embodiment, an antibody comprises a heavy chain constant domain selected from the group consisting of IgG, IgGI, IgG2, IgG2A, IgG2B, IgG2C, IgG3, IgG4, IgAI, IgA2, IgD, IgM, and IgE constant domains. In some embodiments, an antibody comprises a heavy (H) chain variable region (abbreviated herein as VH), and/or (e.g., and) a light (L) chain variable region (abbreviated herein as VL). In some embodiments, an antibody comprises a constant domain, e.g., an Fc region. An immunoglobulin constant domain refers to a heavy or light chain constant domain. Human IgG heavy chain and light chain constant domain amino acid sequences and their functional variations are known. With respect to the heavy chain, in some embodiments, the heavy chain of an antibody described herein can be an alpha (a), delta (D), epsilon (e), gamma (g) or mu (m) heavy chain. In some embodiments, the heavy chain of an antibody described herein can comprise a human alpha (a), delta (D), epsilon (e), gamma (g) or mu (m) heavy chain. In a particular embodiment, an antibody described herein comprises a human gamma 1 CHI, CH2, and/or (e.g., and) CH3 domain. In some embodiments, the amino acid sequence of the VH domain comprises the amino acid sequence of a human gamma (g) heavy chain constant region, such as any known in the art. Non-limiting examples of human constant region sequences have been described in the art, e.g., see U.S. Pat. No. 5,693,780 and Kabat E A et al, (1991) supra. In some embodiments, the VH domain comprises an amino acid sequence that is at least 70%, 75%, 80%, 85%, 90%, 95%, 98%, or at least 99% identical to any of the variable chain constant regions provided herein. In some embodiments, an antibody is modified, e.g., modified via glycosylation, phosphorylation, sumoylation, and/or (e.g., and) methylation. In some embodiments, an antibody is a glycosylated antibody, which is conjugated to one or more sugar or carbohydrate molecules. In some embodiments, the one or more sugar or carbohydrate molecule are conjugated to the antibody via N-glycosylation, O-glycosylation, C-glycosylation, glypiation (GPI anchor attachment), and/or (e.g., and) phosphoglycosylation. In some embodiments, the one or more sugar or carbohydrate molecule are monosaccharides, disaccharides, oligosaccharides, or glycans. In some embodiments, the one or more sugar or carbohydrate molecule is a branched oligosaccharide or a branched glycan. In some embodiments, the one or more sugar or carbohydrate molecule includes a mannose unit, a glucose unit, an N-acetylglucosamine unit, an N-acetylgalactosamine unit, a galactose unit, a fucose unit, or a phospholipid unit. In some embodiments, an antibody is a construct that comprises a polypeptide comprising one or more antigen binding fragments of the disclosure linked to a linker polypeptide or an immunoglobulin constant domain. Linker polypeptides comprise two or more amino acid residues joined by peptide bonds and are used to link one or more antigen binding portions. Examples of linker polypeptides have been reported (see e.g., Holliger, P, et al. (1993) Proc. Natl. Acad. Sci. USA 90:6444-6448; Poljak, R. J., et al. (1994) Structure 2:1121-1123). Still further, an antibody may be part of a larger immunoadhesion molecule, formed by covalent or noncovalent association of the antibody or antibody portion with one or more other proteins or peptides. Examples of such immunoadhesion molecules include use of the streptavidin core region to make a tetrameric scFv molecule (Kipriyanov, S. M., et al. (1995) Human Antibodies and Hybridomas 6:93-101) and use of a cysteine residue, a marker peptide and a C-terminal polyhistidine tag to make bivalent and biotinylated scFv molecules (Kipriyanov, S. M., et al. (1994) Mol. Immunol. 31:1047-1058).

The term “single domain antibody”, or “sdAb”, in short, or ‘nanobody’, has its regular scientific meaning and here refers to an antibody fragment consisting of a single monomeric variable antibody domain, unless referred to as more than one monomeric variable antibody domain such as for example in the context of a bivalent sdAb, which comprises two of such monomeric variable antibody domains in tandem. A bivalent nanobody is a molecule comprising two single domain antibodies targeting epitopes on molecules present at the extracellular side of a cell, such as epitopes on the extracellular domain of a cell surface molecule that is present on the cell. Preferably the cell-surface molecule is a cell-surface receptor. A bivalent nanobody is also named a bivalent single domain antibody. Preferably the two different single domain antibodies are directly covalently bound or covalently bound through an intermediate molecule that is covalently bound to the two different single domain antibodies. Preferably the intermediate molecule of the bivalent nanobody has a molecular weight of less than 10,000 Dalton, more preferably less than 5000 Dalton, even more preferably less than 2000 Dalton, most preferably less than 1500 Dalton.

The term “GalNAc” has its regular scientific meaning and here refers to N-acetylgalactosamine and to the IUPAC name thereof: 2-(acetylamino)-2-deoxy-D-galactose.

The term “(GalNAc)3Tris” has its regular scientific meaning in for example the field of siRNA-based therapy, and here refers to a moiety comprising three GalNAc units each separately covalently bound to the hydroxyl groups of tris(hydroxymethyl)aminomethane (Tris) (IUPAC name: 2-amino-2-(hydroxymethyl) propane-1 3-diol), preferably via at least one linker. (GalNAc)3Tris can exist as a free amine comprising molecule or may be further functionalized via the remaining amine binding site, for example to form the (GalNAc)3Tris-moiety comprising conjugates described herein.

As used herein, the term “covalently linked” refers to a characteristic of two or more molecules being linked together via at least one covalent bond, i.e. directly, or via a chain of covalent bonds, i.e. via a linker comprising at least one or more atoms.

The term “moiety” has its regular scientific meaning and here refers to a molecule that is bound, linked, conjugated to a further molecule, linker, assembly of molecules, etc., and therewith forming part of a larger molecule, conjugate, assembly of molecules. Typically, a moiety is a first molecule that is covalently bound to a second molecule, involving one or more chemical groups initially present on the first and second molecules. For example, when a saponin molecule is covalently linked via at least one linker to one or more GalNAc molecules, both the saponin molecule is a saponin moiety in the formed saponin-GalNAc conjugate and the GalNAc molecule(s) is/are a moiety/moieties in said conjugate. For example, a nucleic acid such as an antisense oligonucleotide, that is conjugated to an endocytic receptor binding ligand such as an antibody or one or more GalNAc molecules, is a nucleic acid moiety in the nucleic acid —GalNAc conjugate.

As used herein, the terms “administering” or “administration” means to provide a complex to a subject in a manner that is physiologically and/or (e.g., and) pharmacologically useful (e.g., to treat a condition in the subject).

As used herein, the term “approximately” or “about,” as applied to one or more values of interest, refers to a value that is similar to a stated reference value. In certain embodiments, the term “approximately” or “about” refers to a range of values that fall within 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

The terms first, second, third and the like in the description and in the claims, are used for distinguishing between for example similar elements, compositions, constituents in a composition, or separate method steps, and not necessarily for describing a sequential or chronological order. The terms are interchangeable under appropriate circumstances and the embodiments of the invention can operate in other sequences than described or illustrated herein, unless specified otherwise.

The term “comprising”, used in the claims, should not be interpreted as being restricted to for example the elements or the method steps or the constituents of a compositions listed thereafter; it does not exclude other elements or method steps or constituents in a certain composition. It needs to be interpreted as specifying the presence of the stated features, integers, (method) steps or components as referred to, but does not preclude the presence or addition of one or more other features, integers, steps or components, or groups thereof. Thus, the scope of the expression “a method comprising steps A and B” should not be limited to a method consisting only of steps A and B, rather with respect to the present invention, the only enumerated steps of the method are A and B, and further the claim should be interpreted as including equivalents of those method steps. Thus, the scope of the expression “a composition comprising components A and B” should not be limited to a composition consisting only of components A and B, rather with respect to the present invention, the only enumerated components of the composition are A and B, and further the claim should be interpreted as including equivalents of those components.

In addition, reference to an element or a component by the indefinite article “a” or “an” does not exclude the possibility that more than one of the element or component are present, unless the context clearly requires that there is one and only one of the elements or components. The indefinite article “a” or “an” thus usually means “at least one”.

The use of terms in brackets in the text, with the exception of chemical and/or mathematical formulae, usually means that the term within brackets specifies a possible option or a possible meaning and should thus not be considered limiting.

The embodiments as described herein can operate in combination and cooperation, unless specified otherwise. Furthermore, the various embodiments, although referred to as “preferred” or “e.g.” or “for example” or “in particular” and the like are to be construed as exemplary manners in which the disclosed herein concepts may be implemented rather than as limiting.

### BRIEF DESCRIPTION OF THE DRAWINGS

FIG. 1: Structure of trivalent GalNAc-oligonucleotide, for example trivalent GalNAc-siRNA also referred to as GN3-siRNA, or in a specific example GN3-siTTR.

FIG. 2A, 2B, 2C, 2D, 2E: Depot release by saponin components in vivo: efficacy and durability of effect (here, serum TTR protein reduction) of co-administration of saponin component (here, GN3-SC-SO1861) and an oligonucleotide, here GN3-siTTR. GN3-siTTR was always administered on day 0 and GN3-SC-SO1861 was administered at the timepoints indicated by the arrow; n=6 mice in all groups except vehicle, where n=3; shown is mean TTR serum level ±SD. (A) GN3-siTTR administered together with saponin component at day 0, (B) GN3-siTTR administered at day 0, saponin component administered at day 7 (arrow), (C) GN3-siTTR administered at day 0, saponin component administered at day 14 (arrow), (D) GN3-siTTR administered at day 0, saponin component administered at day 21 (arrow), (E) GN3-siTTR administered at day 0, saponin component administered at day 28 (arrow).

FIG. 3A, 3B, 3C, 3D, 3E: Depot release of liver targeted oligonucleotides, here GN3-siTTR, by saponin components in vitro measured by reduction in Ttr RNA levels. (A) Incubation scheme of depot release from murine primary hepatocytes (MPH) with saponin components with 6 hr loading of GN3-siTTR, wash out and subsequent release by saponin components (or PBS for a control condition), (B) Ttr RNA quantification following 6 hr loading with GN3-siTTR (wherein the oligonucleotide was designed as an oligonucleotide with advanced enhanced stabilization chemistry (AdvESC) with DV18 chemistry) followed by different saponin components (or PBS) for 24 hrs (C) Ttr RNA quantification following 6 hr loading with GN3-siTTR (wherein the oligonucleotide was designed as an oligonucleotide with advanced enhanced stabilization chemistry (AdvESC) with DV18 chemistry) followed by different saponin components (or PBS) for 48 hrs, (D) Incubation scheme of depot release with saponin components with a 6 hr loading of GN3-siTTR, then wash and resting period in medium, followed by short saponin component pulse for 3 hrs (top scheme) or 6 hrs (bottom scheme), followed by wash and resting period before analysis, (E) Ttr RNA quantification following loading of GN3-siTTR, wash and resting period, and then saponin component pulse for 6 hrs for release, before analysis after an additional 24 hrs.

FIG. 4A, 4B, 4C, 4D, 4E, 4F, 4G, 4H, 4I, 4J, 4K, 4L: Depot release in various cancer cell lines by saponin components in vitro: efficacy and durability enhancement of STAT3 ASO by saponin components measured by STAT3 RNA levels. (A) Incubation scheme of co-administration of STAT3 ASO with saponin components for 48 hrs (positive control), (B) Incubation scheme of depot release with saponin components, by first loading cells with STAT3 ASO followed by wash and release incubation with saponin components for 48 hrs, (C) Incubation scheme of loading with STAT3 ASO, followed by wash and a 6 hr resting period in medium, followed by saponin components for 48 hrs, (D) Incubation scheme of loading with STAT3 ASO, followed by wash and a 24 hr resting period in medium, followed by saponin components for 48 hrs, (E-H) Co-treatment in an EGFR-high expressing (EGFR++) epidermoid carcinoma cell line (A431): STAT3 RNA quantification following co-administration of STAT3 ASO and saponin components (or PBS): (E) for co-administration of STAT3 ASO and saponin component as described in FIG. 4A, (F) for STAT3 ASO loading followed by saponin component as described in FIG. 4B, (G) for STAT3 ASO loading, 6 hrs resting and after that saponin component as described in FIG. 4C, (H) for STAT3 ASO loading, 24 hrs resting and after that saponin component as described in FIG. 4D. (I-L): Co-treatment in a metastatic melanoma cell line (A2058), lacking the EGFR receptor (EGFR-), the target for cetuximab: STAT3 RNA quantification following the timed release by different saponin component treatments (or PBS): (I) for co-administration of STAT3 ASO and saponin component as described under (FIG. 4A), (J) for STAT3 ASO loading followed by saponin component as described in FIG. 4B, (K) for STAT3 ASO loading, 6 hrs resting and after that saponin component as described in FIG. 4C, (L) for STAT3 ASO loading, 24 hrs resting and after that saponin component as described in FIG. 4D.

FIG. 5A, 5B: Visualized gradual depot release by different saponin components in A431 cells. (A) Incubation scheme for imaging gradual depot release with saponin components, by first loading cells with Cy5-labeled cetuximab (Cet-Cy5) for 48 hrs, followed by wash and resting phase in medium for 24 hrs, before release trigger with saponin components, or PBS as control, (B) Quantification of Cy5-release by measuring total integrated Cy5-fluorescence intensity under influence of different saponin components.

FIG. 6A, 6B, 6C, 6D, 6E, 6F, 6G: Depot release of proteinaceous payload (toxin) in epidermoid carcinoma cells by saponin components: in vitro efficacy and durability enhancement of an immunotoxin anti-CD71-saporin (aCD71-SPRN or protein toxin) under influence of saponin components of in A431 cells data. (A) Incubation scheme of co-administration of aCD71-SPRN with saponin components (positive control), (B) Incubation scheme of timed depot release with saponin components. After 24 hr loading with aCD71-SPRN, followed by wash, saponin components were added and cells were analyzed after 48 hrs, (C) Incubation scheme of timed depot release with saponin components after short resting phase: after 24 hr loading with aCD71-SPRN, followed by wash and a 6 hr resting period in medium, saponin components were added and cells were analyzed after 48 hrs, (D) Incubation scheme of depot release with saponin components after longer resting phase: after 24 hr loading with aCD71-SPRN, followed by wash and a 24 hr resting period in medium, saponin components were added and cells were analyzed after 48 hrs, (E) Relative cell viability for the different treatment conditions (FIG. 6A-6D) for a concentration range of saponin component 1 (SO1861) at a fixed concentration of 5 pM aCD71-SPRN in A431 cells, (F) Relative cell viability for the different treatment conditions (FIG. 6A-6D) for a concentration range of saponin component 2 (SO1861-AH-Maleimide-Block, also referred to as Sa1861-AH-Block, a saponin molecule according to formula (V)) at a fixed concentration of 5 pM aCD71-SPRN in A431 cells, (G) Relative cell viability for the different treatment conditions (FIG. 6A-6D) for a concentration range of saponin component 3 (cetuximab-AH-SO1861) at a fixed concentration of 5 pM aCD71-SPRN in A431 cells.

FIG. 7A, 7B: Depot release of proteinaceous payload in a metastatic melanoma cells under influence of saponin components: in vitro efficacy and durability enhancement of of immunotoxin anti-CD71-saporin (aCD71-SPRN) under influence of saponin components in A2058 cells data. (A) Relative cell viability for the different treatment conditions (FIG. 6A-6D) for a concentration range of saponin component 1 (SO1861; see the formula of SO1861 in FIG. 8) at a fixed concentration of 5 pM aCD71-SPRN in A2058 cells, (B) Relative cell viability for the different treatment conditions (FIG. 6A-6D) for a concentration range of saponin component 2 (SO1861-AH-Maleimide-Block, also referred to as SO1861-AH-Block, a saponin molecule according to formula (V) at a fixed concentration of 5 pM aCD71-SPRN in A2058 cells.

FIG. 8: Synthesis and chemical structure of SO1861-AH-azide

FIG. 9: Synthesis and chemical structure of SO1861-SC-azide

FIG. 10: Synthesis and chemical structure of GN3-AH-SO1861

FIG. 11: Synthesis and chemical structure of GN3-SC-SO1861

FIG. 12A, FIG. 12B, FIG. 12C: Efficacy enhancement by saponin compounds at different co-doses (of either 0, 0.3, 1 or 3 mg/kg GN3-AH-SO1861, also referred to as GN3-saponin, or GN3-SPT) an oligonucleotide of 0.3 mg/kg GN3-siAT3 in the non-human primate (NHP). Shown are AT3 serum levels. (A) GN3-siAT3 administered alone, or together with saponin component (at 0.3 mg/kg) at day 0, (B) GN3-siAT3 administered alone at day 0, or together with saponin component (at 1 mg/kg) administered at day 0, (C) GN3-siAT3 administered alone at day 0, or together with saponin component administered (3 mg/kg) at day 0. In all cases, no marked AT3 serum protein reduction is observed when GN3-siAT3 is administered alone, but in combination with saponin compound, efficacy is observed as AT3 protein reduction: here, already 0.3 mg/kg saponin compound show marked efficacy increase that is not further increased by a higher saponin compound dose, indicating that already 0.3 mg/kg is at or above a maximum release efficacy dose.

FIG. 13: Depot release by saponin components in vivo in NHP: efficacy and durability of effect with co-administration of saponin component (here, GN3-AH-SO1861, also referred to as GN3-saponin, or GN3-SPT) and an oligonucleotide, here GN3-siAT3. GN3-siAT3 was administered on day 0 in n=2 NHPs; in one NHP, 0.1 mg/kg saponin compound (in the form of GN3-saponin) was administered at day 28 (indicated by the arrow); shown are AT3 serum levels. Only when the saponin compound was added, a marked decrease in AT3 serum levels was observed, and to levels considered clinically meaningful (between 15-35%, also referred to as the AT3 target range).

### DETAILED DESCRIPTION OF THE INVENTION

### The Saponin Component

The term “saponin component” has its regular scientific meaning and here refers to a component comprising a saponin moiety or consisting of a saponin molecule, wherein the saponin moiety or the saponin molecule is:
a penta-cyclic triterpene saponin of the 12,13-dehydrooleanane type; and
preferably comprising an aldehyde function in position C-23 of the aglycone core structure of the saponin molecule or optionally comprising an aldehyde function in position C-23 of the aglycone core structure of the saponin moiety; and/or
mono-desmosidic or bi-desmosidic, preferably bi-desmosidic; and/or
comprising a first saccharide chain bound to its aglycone core structure, selected from Group A listed in Table 1A and/or comprising a second saccharide chain bound to its aglycone core structure, selected from Group B listed in Table 1 A, and preferably a first saccharide chain and a second saccharide chain are comprised by the saponin molecule or saponin moiety:

TABLE 1A
GLYCANS

Group A
Ara/Xyl-(1→4)-Rha/Fuc-(1→2)-Glc/Gal-(1→2)-Rha/Fuc-(1→2)-GlcA-
Gal-
Gal-(1→2)-[Xyl-(1→3)]-GlcA-
Glc-
Glc-(1→2)-[Glc-(1→4)]-GlcA-
Glc-(1→2)-Ara-(1→3)-[Gal-(1→2)]-GlcA-
GlcA-
Rha-(1→2)-Ara-
Rha-(1→2)-Gal-(1→3)-[Glc-(1→2)]-GlcA-
Xyl-(1→2)-Ara-(1→3)-[Gal-(1→2)]-GlcA-
Group B
[4,6-di-OAc-Glc-(1→3)]-[Xyl-(1→4)]-Rha-(1→2)-[3,4-di-OAc-Qui-(1→4)]-Fuc-
6-OAc-Glc-(1→3)-[Xyl-(1→4)]-Rha-(1→2)-Fuc-
6-OAc-Glc-(1→3)-Xyl-(1→4)-Rha-(1→2)-[3-OAc-Rha-(1→3)]-Fuc-
Api-(1→3)-Xyl-(1→4)-[Glc-(1→3)]-Rha-(1→2)-[R-(→4)]-Fuc-
wherein R is 5-O-[5-O-Ara/Api-3,5-dihydroxy-6-methyl-octanoyl]-3,5-dihydroxy-6-methyl-
octanoic acid
Api-(1→3)-Xyl-(1→4)-[Glc-(1→3)]-Rha-(1→2)-[Rha-(1→3)]-4OAc-Fuc-
Api-(1→3)-Xyl-(1→4)-[Glc-(1→3)]-Rha-(1→2)-[Rha-(1→3)]-4-OAc-Fuc-
Api-(1→3)-Xyl-(1→4)-[Glc-(1→3)]-Rha-(1→2)-Fuc-
Api-(1→3)-Xyl-(1→4)-Rha-(1→2)-[R-(→3)]-Fuc-
wherein R is 5-O-[5-O-Ara/Api-3,5-dihydroxy-6-methyl-octanoyl]-3,5-dihydroxy-6-methyl-
octanoic acid
Api-(1→3)-Xyl-(1→4)-Rha-(1→2)-[R-(→4)]-Fuc-
wherein R is 5-O-[5-O-Ara/Api-3,5-dihydroxy-6-methyl-octanoyl]-3,5-dihydroxy-6-methyl-
octanoic acid
Api-(1→3)-Xyl-(1→4)-Rha-(1→2)-[Rha-(1→3)]-4-OAc-Fuc-
Api/Xyl-(1→3)-Xyl-(1→4)-[Glc-(1→3)]-Rha-(1→2)-[R-(→4)]-Fuc-
wherein R is 5-O-[5-O-Ara/Api-3,5-dihydroxy-6-methyl-octanoyl]-3,5-dihydroxy-6-methyl-
octanoic acid
Api/Xyl-(1→3)-Xyl-(1→4)-[Glc-(1→3)]-Rha-(1→2)-[R-(→4)]-Fuc-
wherein R is 5-O-[5-O-Rha-(1→2)-Ara/Api-3,5-dihydroxy-6-methyl-octanoyl]-3,5-dihydroxy-
6-methyl-octanoic acid
Api/Xyl-(1→3)-Xyl-(1→4)-[Glc-(1→3)]-Rha-(1→2)-[Rha-(1→3)]-4OAc-Fuc-
Ara/Xyl-
Ara/Xyl-(1→3)-Ara/Xyl-(1→4)-Rha/Fuc-(1→2)-[4-OAc-Rha/Fuc-(1→4)]-Rha/Fuc-
Ara/Xyl-(1→4)-Rha/Fuc-(1→4)-[Glc/Gal-(1→2)]-Fuc-
Glc-(1→3)-[Glc-(1→6)]-Gal-
Glc-(1→3)-[Xyl-(1→3)-Xyl-(1→4)]-Rha-(1→2)-Fuc-
Glc-(1→3)-[Xyl-(1→4)]-Rha-(1→2)-[Qui-(1→4)]-Fuc-
Glc-(1→3)-[Xyl-(1→4)]-Rha-(1→2)-Fuc-
Glc-(1→3)-Xyl-(1→4)-[Glc-(1→3)]-Rha-(1→2)-4-OAc-Fuc-
Glc-(1→3)-Xyl-(1→4)-Rha-(1→2)-[3-OAc--Rha-(1→3)]-Fuc-
Glc-(1→3)-Xyl-(1→4)-Rha-(1→2)-[Xyl-(1→3)-4-OAc-Qui-(1→4)]-Fuc-
Glc-(1→3)-Xyl-(1→4)-Rha-(1→2)-4-OAc-Fuc-
Glc/Gal-
Rha-(1→2)-[Ara-(1→3)-Xyl-(1→4)]-Rha-
Rha-(1→2)-[Xyl-(1→4)]-Rha-
Xyl-(1→3)-Xyl-(1→4)-Rha-(1→2)-[3,4-di-OAc-Qui-(1→4)]-Fuc-
Xyl-(1→3)-Xyl-(1→4)-Rha-(1→2)-[Qui-(1→4)]-Fuc-
Xyl-(1→3)-Xyl-(1→4)-Rha-(1→2)-[R-(→3)]-Fuc-
wherein R is 5-O-[5-O-Ara/Api-3,5-dihydroxy-6-methyl-octanoyl]-3,5-dihydroxy-6-methyl-
octanoic acid
Xyl-(1→3)-Xyl-(1→4)-Rha-(1→2)-[R-(→4)]-Fuc-
wherein R is 5-O-[5-O-Ara/Api-3,5-dihydroxy-6-methyl-octanoyl]-3,5-dihydroxy-6-methyl-
octanoic acid
Xyl-(1→3)-Xyl-(1→4)-Rha-(1→2)-[Xyl-(1→3)-4-OAc-Qui-(1→4)]-Fuc-
Xyl-(1→4)-[Gal-(1→3)]-Rha-(1→2)-4-OAc-Fuc-
Xyl-(1→4)-[Gal-(1→3)]-Rha-(1→2)-Fuc-
Xyl-(1→4)-[Glc-(1→3)]-Rha-(1→2)-[R-(→4)]-3-OAc-Fuc-
wherein R is 4E-Methoxycinnamic acid)
Xyl-(1→4)-[Glc-(1→3)]-Rha-(1→2)-3,4-di-OAc-Fuc-
Xyl-(1→4)-[Glc-(1→3)]-Rha-(1→2)-Fuc-
Xyl-(1→4)-Rha-(1→2)-[R-(→4)]-Fuc-
wherein R is 4E-Methoxycinnamic acid
Xyl-(1→4)-Rha-(1→2)-[R-(→4)]-Fuc-
wherein R is 4Z-Methoxycinnamic acid

and/or
preferably comprising a first saccharide chain bound to position C-3 of its aglycone core structure, selected from Group A listed in Table 1A, wherein preferably said first saccharide chain of the saponin molecule comprises a glucuronic acid group or optionally said first saccharide chain of the saponin moiety comprises a glucuronic acid group; and/or
preferably comprising the first saccharide chain which comprises a terminal glucuronic acid residue and/or comprising the second saccharide chain which comprises at least four sugar residues in a branched configuration; and/or
preferably comprising the first saccharide chain Gal-(1→2)-[Xyl-(1→3)]-GIcA and/or a branched second saccharide chain of at least four sugar residues comprising a terminal fucose residue and/or a terminal rhamnose residue, preferably selected from Table 1 A; and/or
preferably comprising a first saccharide chain at position C-3 of the saponin's aglycone core structure and/or a second saccharide chain at position C-28 of the saponin's aglycone core structure, preferably wherein the first saccharide chain is a carbohydrate substituent at the C-3beta-OH group of the saponin's aglycone core structure and/or wherein the second saccharide chain is a carbohydrate substituent at the C-28-OH group of the saponin's aglycone core structure; and/or
optionally comprising at least one acetoxy (Me(CO)O-) group in the first saccharide chain and/or in the second saccharide chain, preferably in the second saccharide chain; and/or
comprising an aglycone core structure selected from:
quillaic acid;
gypsogenin;
2alpha-hydroxy oleanolic acid;
16alpha-hydroxy oleanolic acid;
hederagenin (23-hydroxy oleanolic acid);
16alpha,23-dihydroxy oleanolic acid;
protoaescigenin-21(2-methylbut-2-enoate)-22-acetate;
23-oxo-barringtogenol C-21,22-bis(2-methylbut-2-enoate);
23-oxo-barringtogenol C-21(2-methylbut-2-enoate)-16,22-diacetate;
3,16,28-trihydroxyoleanan-12-en;
gypsogenic acid; and
a derivative thereof; and/or
preferably comprising an aglycone core structure selected from quillaic acid, gypsogenin, and a derivative thereof; and/or
preferably comprising the aglycone core structure quillaic acid; and/or
selected from any one or more of the saponins listed in Table 2A:

TABLE 2A
Saponins displaying (late) endosomal/lysosomal escape enhancing activity,
and with a aglycone core of the 12,13-dehydrooleanane type⁴⁾

| | Aglycone core | Carbohydrate | |
|---|---|---|---|
| | with an aldehyde | substituent at the | |
| | group at the C-23 | C-3beta-OH | Carbohydrate substituent at the C-28- |
| Saponin Name | position | group | OH group |
| NP-017777 | Gypsogenin | Gal-(1→2)-[Xyl- | Xyl-(1→4)-Rha-(1→2)-[R-(→4)]-Fuc- (R = |
| | | (1→3)]-GlcA- | 4E-Methoxycinnamic acid) |
| NP-017778 | Gypsogenin | Gal-(1→2)-[Xyl- | Xyl-(1→4)-Rha-(1→2)-[R-(→4)]-Fuc- (R = |
| | | (1→3)]-GlcA- | 4Z-Methoxycinnamic acid) |
| NP-017774 | Gypsogenin | Gal-(1→2)-[Xyl- | Xyl-(1→4)-[Gal-(1→3)]-Rha-(1→2)-4-OAc- |
| | | (1→3)]-GlcA- | Fuc- |
| NP-018110^{c}, | Gypsogenin | Gal-(1→2)-[Xyl- | Xyl-(1→4)-[Glc-(1→3)]-Rha-(1→2)-3,4-di- |
| NP-017772^{d} | | (1→3)]-GlcA- | OAc-Fuc- |
| NP-018109 | Gypsogenin | Gal-(1→2)-[Xyl- | Xyl-(1→4)-[Glc-(1→3)]-Rha-(1→2)-[R- |
| | | (1→3)]-GlcA- | (→4)]-3-OAc-Fuc- (R = 4E- |
| | | | Methoxycinnamic acid) |
| NP-017888 | Gypsogenin | Gal-(1→2)-[Xyl- | Glc-(1→3)-Xyl-(1→4)-[Glc-(1→3)]-Rha- |
| | | (1→3)]-GlcA- | (1→2)-4-OAc-Fuc- |
| NP-017889 | Gypsogenin | Gal-(1→2)-[Xyl- | Glc-(1→3)-Xyl-(1→4)-Rha-(1→2)-4-OAc- |
| | | (1→3)]-GlcA- | Fuc- |
| NP-018108 | Gypsogenin | Gal-(1→2)-[Xyl- | Ara/Xyl-(1→3)-Ara/Xyl-(1→4)-Rha/Fuc- |
| | | (1→3)]-GlcA- | (1→2)-[4-OAc-Rha/Fuc-(1→4)]-Rha/Fuc- |
| SA1641^{a}, | Gypsogenin | Gal-(1→2)-[Xyl- | Xyl-(1→3)-Xyl-(1→4)-Rha-(1→2)-[Qui- |
| AEX55^{b} | | (1→3)]-GlcA- | (1→4)]-Fuc- |
| SO1658 | Gypsogenin | Gal-(1→2)-[Xyl- | Glc-(1→3)-[Xyl-(1→3)-Xyl-(1→4)]-Rha- |
| | | (1→3)]-GlcA- | (1→2)-Fuc- |
| gypsoside A⁶⁾ | Gypsogenin | Gal-(1→4)-Glc | Xyl-(1→3)-Fuc-(1→4)-[Xyl-(1→3)-Xyl- |
| | | (1→4)-[Ara- | (1→3)]-Rha- |
| | | (1→3)]-GlcA- | |
| phytolaccagenin | Gypsogenin | Absent | absent |
| Gypsophila | Quillaic acid | Gal-(1→2)-[Xyl- | Glc-(1→3)-[Xyl-(1→4)]-Rha-(1→2)-Fuc- |
| saponin 1 (Gyp1) | | (1→3)]-GlcA- | |
| NP-017674 | Quillaic acid | Gal-(1→2)-[Xyl- | Api-(1→3)-Xyl-(1→4)-[Glc-(1→3)]-Rha- |
| | | (1→3)]-GlcA- | (1→2)-Fuc- |
| NP-017810 | Quillaic acid | Gal-(1→2)-[Xyl- | Xyl-(1→4)-[Gal-(1→3)]-Rha-(1→2)-Fuc- |
| | | (1→3)]-GlcA- | |
| AG1 | Quillaic acid | Gal-(1→2)-[Xyl- | Xyl-(1→4)-[Glc-(1→3)]-Rha-(1→2)-Fuc- |
| | | (1→3)]-GlcA- | |
| NP-003881 | Quillaic acid | Gal-(1→2)-[Xyl- | Ara/Xyl-(1→4)-Rha/Fuc-(1→4)-[Glc/Gal- |
| | | (1→3)]-GlcA- | (1→2)]-Fuc- |
| NP-017676 | Quillaic acid | Gal-(1→2)-[Xyl- | Api-(1→3)-Xyl-(1→4)-[Glc-(1→3)]-Rha- |
| | | (1→3)]-GlcA- | (1→2)-[R-(→4)]-Fuc- |
| | | | (R = 5-O-[5-O-Ara/Api-3,5-dihydroxy-6- |
| | | | methyl-octanoyl]-3,5-dihydroxy-6-methyl- |
| | | | octanoic acid) |
| NP-017677 | Quillaic acid | Gal-(1→2)-[Xyl- | Api-(1→3)-Xyl-(1→4)-Rha-(1→2)-[R-(→4)]- |
| | | (1→3)]-GlcA- | Fuc- |
| | | | (R = 5-O-[5-O-Ara/Api-3,5-dihydroxy-6- |
| | | | methyl-octanoyl]-3,5-dihydroxy-6-methyl- |
| | | | octanoic acid) |
| NP-017706 | Quillaic acid | Gal-(1→2)-[Xyl- | Api-(1→3)-Xyl-(1→4)-Rha-(1→2)-[Rha- |
| | | (1→3)]-GlcA- | (1→3)]-4-OAc-Fuc- |
| NP-017705 | Quillaic acid | Gal-(1→2)-[Xyl- | Api-(1→3)-Xyl-(1→4)-[Glc-(1→3)]-Rha- |
| | | (1→3)]-GlcA- | (1→2)-[Rha-(1→3)]-4-OAc-Fuc- |
| NP-017773 | Quillaic acid | Gal-(1→2)-[Xyl- | 6-OAc-Glc-(1→3)-Xyl-(1→4)-Rha-(1→2)- |
| | | (1→3)]-GlcA- | [3-OAc-Rha-(1→3)]-Fuc- |
| NP-017775 | Quillaic acid | Gal-(1→2)-[Xyl- | Glc-(1→3)-Xyl-(1→4)-Rha-(1→2)-[3-OAc-- |
| | | (1→3)]-GlcA- | Rha-(1→3)]-Fuc- |
| SA1657 | Quillaic acid | Gal-(1→2)-[Xyl- | Xyl-(1→3)-Xyl-(1→4)-Rha-(1→2)-[Qui- |
| | | (1→3)]-GlcA- | (1→4)]-Fuc- |
| AG2 | Quillaic acid | Gal-(1→2)-[Xyl- | Glc-(1→3)-[Xyl-(1→4)]-Rha-(1→2)-[Qui- |
| | | (1→3)]-GlcA- | (1→4)]-Fuc- |
| GE1741 | Quillaic acid | Gal-(1→2)-[Xyl- | Xyl-(1→3)-Xyl-(1→4)-Rha-(1→2)-[3,4-di- |
| | | (1→3)]-GlcA- | OAc-Qui-(1→4)]-Fuc- |
| SO1542 | Quillaic acid | Gal-(1→2)-[Xyl- | Glc-(1→3)-[Xyl-(1→4)]-Rha-(1→2)-Fuc- |
| | | (1→3)]-GlcA- | |
| SO1584 | Quillaic acid | Gal-(1→2)-[Xyl- | 6-OAc-Glc-(1→3)-[Xyl-(1→4)]-Rha-(1→2)- |
| | | (1→3)]-GlcA- | Fuc- |
| SO1674 | Quillaic acid | Gal-(1→2)-[Xyl- | Glc-(1→3)-[Xyl-(1→3)-Xyl-(1→4)]-Rha- |
| | | (1→3)]-GlcA- | (1→2)-Fuc- |
| SO1700³⁾ | Quillaic acid | Gal-(1→2)-[Xyl- | Xyl-(1→4)-Rha-(1→2)-[Xyl-(1→3)-4-OAc- |
| | | (1→3)]-GlcA- | Qui-(1→4)]-Fuc- |
| Saponarioside B¹⁾ | Quillaic acid | Gal-(1→2)-[Xyl- | Xyl-(1→3)-Xyl-(1→4)-Rha-(1→2)-[4-OAc- |
| | | (1→3)]-GlcA- | Qui-(1→4)]-Fuc- |
| SO1730³⁾ | Quillaic acid | Gal-(1→2)-[Xyl- | Glc-(1→3)-Xyl-(1→4)-Rha-(1→2)-[-4-OAc- |
| | | (1→3)]-GlcA- | Qui-(1→4)]-Fuc- |
| SO1772³⁾ | Quillaic acid | Gal-(1→2)-[Xyl- | 6-OAc-Glc-(1→3)-[Xyl-(1→4)]-Rha-(1→2)- |
| | | (1→3)]-GlcA- | [4-OAc-Qui-(1→4)]-Fuc-- |
| SO1832¹⁾ | Quillaic acid | Gal-(1→2)-[Xyl- | Xyl-(1→3)-Xyl-(1→4)-Rha-(1→2)-[Xyl- |
| (protonated | | (1→3)]-GlcA- | (1→3)-4-OAc-Qui-(1→4)]-Fuc- |
| SO1831) = | | | |
| Saponarioside A | | | |
| SO1861 | Quillaic acid | Gal-(1→2)-[Xyl- | Glc-(1→3)-Xyl-(1→4)-Rha-(1→2)-[Xyl- |
| (deprotonated | | (1→3)]-GlcA- | (1→3)-4-OAc-Qui-(1→4)]-Fuc- |
| SO1862) | | | |
| SO1862 | Quillaic acid | Gal-(1→2)-[Xyl- | Glc-(1→3)-Xyl-(1→4)-Rha-(1→2)-[Xyl- |
| (protonated | | (1→3)]-GlcA- | (1→3)-4-OAc-Qui-(1→4)]-Fuc- |
| SO1861), also | | | |
| referred to as | | | |
| Sapofectosid⁵⁾ | | | |
| SO1904³⁾ | Quillaic acid | Gal-(1→2)-[Xyl- | 6-OAc-Glc-(1→3)-[Xyl-(1→4)]-Rha-(1→2)- |
| | | (1→3)]-GlcA- | [Xyl-(1→3)-4-OAc-Qui-(1→4)]-Fuc- |
| QS-7 (also | Quillaic acid | Gal-(1→2)-[Xyl- | Api/Xyl-(1→3)-Xyl-(1→4)-[Glc-(1→3)]-Rha- |
| referred to as | | (1→3)]-GlcA- | (1→2)-[Rha-(1→3)]-4OAc-Fuc- |
| QS1861) | | | |
| QS-7 api (also | Quillaic acid | Gal-(1→2)-[Xyl- | Api-(1→3)-Xyl-(1→4)-[Glc-(1→3)]-Rha- |
| referred to as | | (1→3)]-GlcA- | (1→2)-[Rha-(1→3)]-4OAc-Fuc- |
| QS1862) | | | |
| QS-17 | Quillaic acid | Gal-(1→2)-[Xyl- | Api/Xyl-(1→3)-Xyl-(1→4)-[Glc-(1→3)]-Rha- |
| | | (1→3)]-GlcA- | (1→2)-[R-(→4)]-Fuc- |
| | | | (R = 5-O-[5-O-Rha-(1→2)-Ara/Api-3,5- |
| | | | dihydroxy-6-methyl-octanoyl]-3,5- |
| | | | dihydroxy-6-methyl-octanoic acid) |
| QS-18 | Quillaic acid | Gal-(1→2)-[Xyl- | Api/Xyl-(1→3)-Xyl-(1→4)-[Glc-(1→3)]-Rha- |
| | | (1→3)]-GlcA- | (1→2)-[R-(→4)]-Fuc- |
| | | | (R = 5-O-[5-O-Ara/Api-3,5-dihydroxy-6- |
| | | | methyl-octanoyl]-3,5-dihydroxy-6-methyl- |
| | | | octanoic acid) |
| QS-21 A-apio | Quillaic acid | Gal-(1→2)-[Xyl- | Api-(1→3)-Xyl-(1→4)-Rha-(1→2)-[R-(→4)]- |
| | | (1→3)]-GlcA- | Fuc- |
| | | | (R = 5-O-[5-O-Ara/Api-3,5-dihydroxy-6- |
| | | | methyl-octanoyl]-3,5-dihydroxy-6-methyl- |
| | | | octanoic acid) |
| QS-21 A-xylo | Quillaic acid | Gal-(1→2)-[Xyl- | Xyl-(1→3)-Xyl-(1→4)-Rha-(1→2)-[R-(→4)]- |
| | | (1→3)]-GlcA- | Fuc- |
| | | | (R = 5-O-[5-O-Ara/Api-3,5-dihydroxy-6- |
| | | | methyl-octanoyl]-3,5-dihydroxy-6-methyl- |
| | | | octanoic acid) |
| QS-21 B-apio | Quillaic acid | Gal-(1→2)-[Xyl- | Api-(1→3)-Xyl-(1→4)-Rha-(1→2)-[R-(→3)]- |
| | | (1→3)]-GlcA- | Fuc- |
| | | | (R = 5-O-[5-O-Ara/Api-3,5-dihydroxy-6- |
| | | | methyl-octanoyl]-3,5-dihydroxy-6-methyl- |
| | | | octanoic acid) |
| QS-21 B-xylo | Quillaic acid | Gal-(1→2)-[Xyl- | Xyl-(1→3)-Xyl-(1→4)-Rha-(1→2)-[R-(→3)]- |
| | | (1→3)]-GlcA- | Fuc- |
| | | | (R = 5-O-[5-O-Ara/Api-3,5-dihydroxy-6- |
| | | | methyl-octanoyl]-3,5-dihydroxy-6-methyl- |
| | | | octanoic acid) |
| QS-21 | Quillaic acid | Gal-(1→2)-[Xyl- | Combination of the carbohydrate chains |
| | | (1→3)]-GlcA- | depicted for QS-21 A-apio, A-xylo, B-apio, |
| | | | B-xylo, for this position at the aglycone |
| | | | (see also the structure depicted as |
| | | | (Scheme Q)) |
| Agrostemmoside E | Quillaic acid | Gal-(1→2)-[Xyl- | [4,6-di-OAc-Glc-(1→3)]-[Xyl-(1→4)]-Rha- |
| (AG1856, AG2.8)²⁾ | | (1→3)]-GlcA- | (1→2)-[3,4-di-OAc-Qui-(1→4)]-Fuc- |

| | Aglycone core | | |
|---|---|---|---|
| | without an | | |
| | aldehyde group at | | Carbohydrate substituent at the C-28- |
| Saponin Name | the C-23 position | | OH group |
| | | Carbohydrate substituent | |
| | | at the C-3beta-OH group | |
| NP-005236 | 2alpha- | GlcA- | Glc/Gal- |
| | Hydroxyoleanolic | | |
| | acid | | |
| AMA-1 | 16alpha- | Glc- | Rha-(1→2)-[Xyl-(1→4)]-Rha- |
| | Hydroxyoleanolic | | |
| | acid | | |
| AMR | 16alpha- | Glc- | Rha-(1→2)-[Ara-(1→3)-Xyl-(1→4)]-Rha- |
| | Hydroxyoleanolic | | |
| | acid | | |
| alpha-Hederin | Hederagenin (23- | Rha-(1→2)-Ara- | Not present |
| | Hydroxyoleanolic | | |
| | acid) | | |
| NP-012672 | 16alpha,23- | Ara/Xyl-(1→4)- | Ara/Xyl- |
| | Dihydroxyoleanolic | Rha/Fuc-(1→2)- | |
| | acid | Glc/Gal-(1→2)- | |
| | | Rha/Fuc-(1→2)- | |
| | | GlcA- | |
| beta-Aescin | Protoaescigenin- | Glc-(1→2)-[Glc- | Not present |
| (described: | 21(2-methylbut-2- | (1→4)]-GlcA- | |
| Aescin Ia) | enoate)-22-acetat | | |
| Aescinate⁷⁾ | Aglycone core | present | Not present |
| | without an aldehyde | | |
| | group at the C-23 | | |
| | position | | |
| dipsacoside B⁸⁾ | Aglycone core | present | present |
| | without an aldehyde | | |
| | group at the C-23 | | |
| | position | | |
| esculentoside A⁹⁾ | Aglycone core | present | Not present |
| | without an aldehyde | | |
| | group at the C-23 | | |
| | position | | |
| Teaseed saponin I | 23-Oxo- | Glc-(1→2)-Ara- | Not present |
| | barringtogenol C - | (1→3)-[Gal- | |
| | 21,22-bis(2- | (1→2)]-GlcA- | |
| | methylbut-2-enoate) | | |
| Teaseedsaponin J | 23-Oxo- barringtogenol C - | Xyl-(1→2)-Ara- | Not present |
| | 21,22-bis(2- | (1→3)-[Gal- | |
| | methylbut-2-enoate) | (1→2)]-GlcA- | |
| Assamsaponin F | 23-Oxo- | Glc-(1→2)-Ara- | Not present |
| | barringtogenol C - | (1→3)-[Gal- | |
| | 21(2-methylbut-2- | (1→2)]-GlcA- | |
| | enoate)-16,22- | | |
| | diacetat | | |
| Primula acid 1 | 3,16,28- | Rha-(1→2)-Gal- | Not present |
| | Trihydroxyoleanan- | (1→3)-[Glc- | |
| | 12-en | (1→2)]-GlcA- | |
| AS64R | Gypsogenic acid | Absent | Glc-(1→3)-[Glc-(1→6)]-Gal- |
| Macranthoidin | Aglycone core | present | present |
| A¹⁰⁾ | without an aldehyde | | |
| | group at the C-23 | | |
| | position | | |
| saikosaponin A¹¹⁾ | Aglycone core | present | absent |
| | without an aldehyde | | |
| | group at the C-23 | | |
| | position | | |
| saikosaponin D¹²⁾ | Aglycone core | present | absent |
| | without an aldehyde | | |
| | group at the C-23 | | |
| | position | | |
| | | Carbohydrate substituent | |
| | | at the C-23-OH group | |
| AS6.2 | Gypsogenic acid | Gal- | Glc-(1→3)-[Glc-(1→6)]-Gal- |

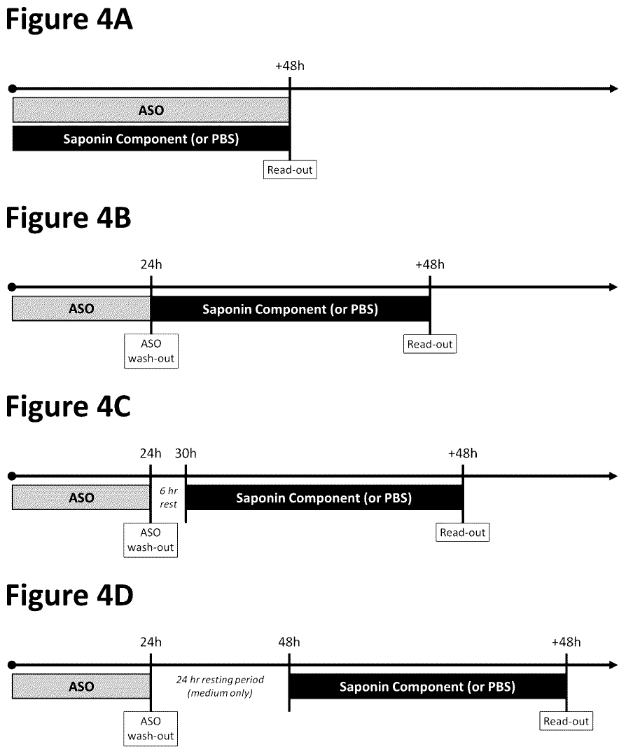

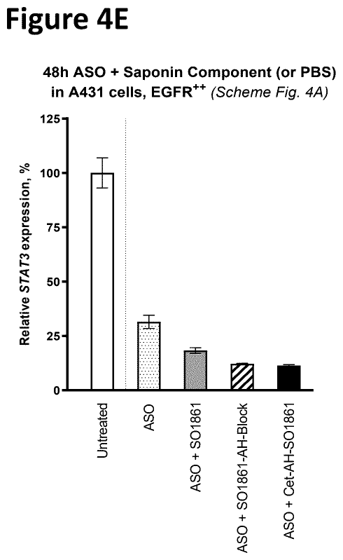

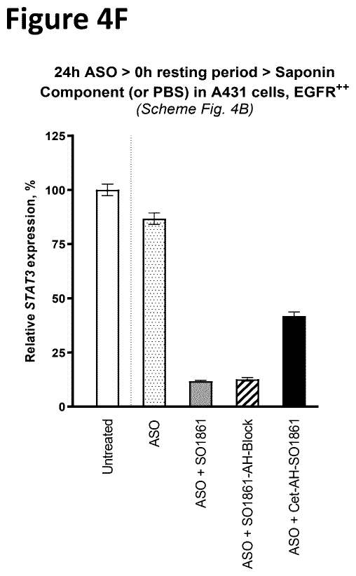

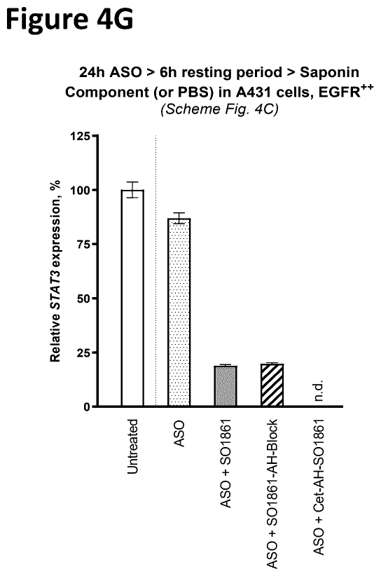

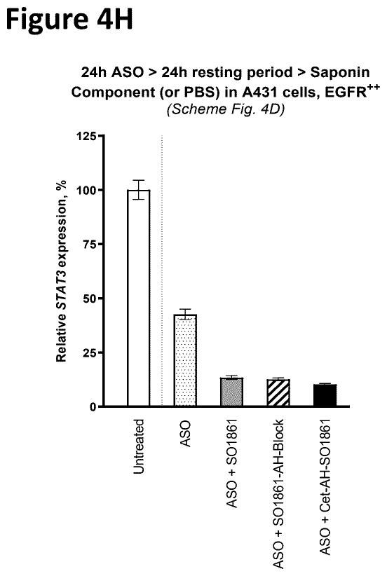

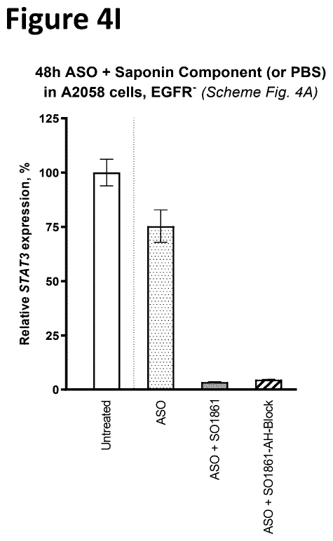

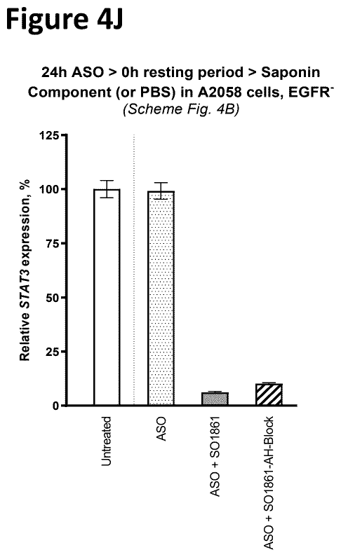

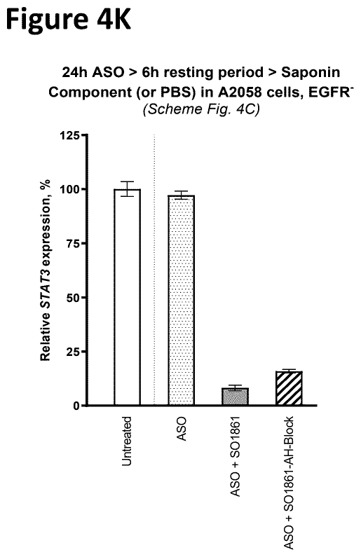

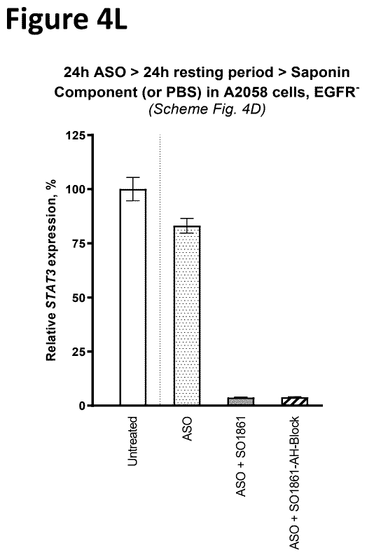

^{a}, ^{b}Different names refer to different isolates of the same structure
^{c}, ^{d}Different names refer to different isolates of the same structure
¹⁾Jia et al., Major Triterpenoid Saponins from Saponaria officinalis, J. Nat. Prod. 1998, 61, 11, 1368-1373, Publication Date: Sep. 19, 1998, https://doi.org/10.1021/np980167u
²⁾The structure of Agrostemmoside E (also referred to as AG1856 or AG2.8) is given in FIG. 4 of J. Clochard et al, A new acetylated triterpene saponin from Agrostemma githago L. modulates gene delivery efficiently and shows a high cellular tolerance, International Journal of Pharmaceutics, Volume 589, 15 Nov. 2020, 119822.
³⁾Structures of SO1700, SO1730, SO1772, SO1904 are given in Moniuszko-Szajwaj et al., Highly Polar Triterpenoid Saponins from the Roots of Saponaria officinalis L., Helv. Chim. Acta, V99, pp. 347-354, 2016 (doi.org/10.1002/hlca.201500224).
⁴⁾See for example:
thesis by Dr Stefan Böttger (2013): Untersuchungen zur synergistischen Zytotoxizität zwischen Saponinen und Ribosomen inaktivierenden Proteinen Typ I; and
Sama et al., Structure-Activity Relationship of Transfection-Modulating Saponins - A Pursuit for the Optimal Gene Trafficker, Planta Med. Volume 85, pp. 513-518, 2019 (doi: 10.1055/a-0863-4795); and
Fuchs et al., Glycosylated Triterpenoids as Endosomal Escape Enhancers in Targeted Tumor Therapies, Biomedicine, Volume 5, issue 14, 2017 (doi: 10.3390/biomedicines5020014).
⁵⁾Sama et al., Sapofectosid - Ensuring non-toxic and effective DNA and RNA delivery, International Journal of Pharmaceutics, Volume 534, Issues 1-2, 20 Dec. 2017, Pages 195-205 (dx.doi.org/10.1016/j.ijpharm.2017.10.016) & Moniuszko-Szajwaj et al., Highly Polar Triterpenoid Saponins from the Roots of Saponaria officinalis L., Helv. Chim. Acta, V99, pp. 347-354, 2016 (doi.org/10.1002/hlca.201500224).
⁶⁾See for example: doi: 10.1016/s0040-4039(01)90658-6, Tetrahedron Letters No. 8, pp. 477-482, 1963 and and “Gipsoside.” National Center for Biotechnology Information. PubChem Compound Database, U.S. National Library of Medicine, 8 Aug. 2005, pubchem.ncbi.nlm.nih.gov/compound/Gipsoside.
⁷⁾The structure of Sodium Aescinate is for example given in the National Library of Medicine PubChem Compound Database (“Sodium Aescinate.” National Center for Biotechnology Information. PubChem Compound Database, U.S. National Library of Medicine, 26 Mar. 2005, pubchem.ncbi.nlm.nih.gov/compound/Sodium-aescinate,). The structure of Aescin (also referred to as Escin) is for example given in the National Library of Medicine PubChem Compound Database (“Escin.” National Center for Biotechnology Information. PubChem Compound Database, U.S. National Library of Medicine, 12 Jul. 2007, pubchem.ncbi.nlm.nih.gov/compound/16211024#section=Other-Identifiers.)
⁸⁾The structure of Dipsacoside B is for example given in the National Library of Medicine PubChem Compound Database (“Dipsacoside B.” National Center for Biotechnology Information. PubChem Compound Database, U.S. National Library of Medicine, 5 Dec. 2007, pubchem.ncbi.nlm.nih.gov/compound/21627940.)
⁹⁾The structure of Esculentoside A is for example given in the National Library of Medicine PubChem Compound Database (“Esculentoside a.” National Center for Biotechnology Information. PubChem Compound Database, U.S. National Library of Medicine, 26 Oct. 2006, pubchem.ncbi.nlm.nih.gov/compound/11657924.)
¹⁰⁾The structure of Macranthoidin A is for example given in the National Library of Medicine PubChem Compound Database (“Macranthoidin a.” National Center for Biotechnology Information. PubChem Compound Database, U.S. National Library of Medicine, 9 Feb. 2007, pubchem.ncbi.nlm.nih.gov/compound/14564503.)
¹¹⁾The structure of Saikosaponin A is for example given in the National Library of Medicine PubChem Compound Database (“Saikosaponin a.” National Center for Biotechnology Information. PubChem Compound Database, U.S. National Library of Medicine, 26 Jun. 2005, pubchem.ncbi.nlm.nih.gov/compound/167928.)
¹²⁾The structure of Saikosaponin D is for example given in the National Library of Medicine PubChem Compound Database (“Saikosaponin d.” National Center for Biotechnology Information. PubChem Compound Database, U.S. National Library of Medicine, 1 Aug. 2005, pubchem.ncbi.nlm.nih.gov/compound/107793.)

and/or
a) selected from any one or more of list A:
   Quillaja saponaria saponin mixture, or a saponin isolated from Quillaja saponaria, for example Quil-A, QS-17-api, QS-17-xyl, QS-21, QS-21A, QS-21B, QS-7-xyl;
   Saponinum album saponin mixture, or a saponin isolated from Saponinum album;
   Saponaria officinalis saponin mixture, or a saponin isolated from Saponaria officinalis; and
   Quillaja bark saponin mixture, or a saponin isolated from Quillaja bark, for example Quil-A, QS-17-api, QS-17-xyl, QS-21, QS-21A, QS-21B, QS-7-xyl; or
b) comprising a gypsogenin aglycone core structure and is selected from list B:
   SA1641, gypsoside A, NP-017772, NP-017774, NP-017777, NP-017778, NP-018109, NP-017888, NP-017889, NP-018108, S01658 and Phytolaccagenin; or
c) comprising a quillaic acid aglycone core structure and is selected from list C:
   AG1856, AG1, AG2, Agrostemmoside E, GE1741, Gypsophila saponin 1 (Gyp1), NP-017674, NP-017810, NP-003881, NP-017676, NP-017677, NP-017705, NP-017706, NP-017773, NP-017775, SA1657, Saponarioside B, S01542, S01584, S01674, S01700, S01730, S01772, SO1 832, Sa1 861, SO1862, S01904, QS-7, QS-7 api, QS-17, QS-18, QS-21 A-apio, QS-21 A-xylo, QS-21 B-apio and QS-21 B-xylo; or
d) comprising a 12, 13-dehydrooleanane type aglycone core structure without an aldehyde group at the C-23 position of the aglycone and is selected from list D:
   Aescin la, aescinate, alpha-Hederin, AMA-1, AMR, AS6.2, AS64R, Assamsaponin F, dipsacoside B, esculentoside A, macranthoidin A, NP-005236, NP-012672, Primula acid 1, saikosaponin A, saikosaponin D, Teaseed saponin I and Teaseedsaponin J,
      preferably, any one or more selected from list A, B or C, more preferably, selected from list B or C, even more preferably selected from list C; and/or
any one or more of AG1856, GE1741, a saponin isolated from Quillaja saponaria, Quil-A, QS-17, QS-21, QS-7, SA1641, a saponin isolated from Saponaria officinalis, Saponarioside B, SO1 542, SO1584, SO1658, SO1674, SO1700, SO1730, SO1772, SO1832, SO1861, SO1862 and S01904, preferably any one or more of QS-21, S01832, SO1861, SA1641, AG1856 and GE1741, more preferably AG1856, S01832 or SO1861, most preferably SO1861 or S01832; and/or
a saponin isolated from Saponaria officinalis, preferably any one or more of Saponarioside B, S01542, S01584, S01658, S01674, S01700, S01730, S01772, S01832, SO1861, SO1862 and SO1 904, more preferably any one or more of SO1 832, SO1861 and SO1 862, even more preferably SO1 832 or SO1 861, most preferably SO1 861; and/or
a saponin molecule, wherein the carboxyl group of the glucuronic acid unit in the first saccharide chain bound to C-3 of the aglycone core structure of the saponin molecule is transformed into an amide bond through reaction with 2-amino-2-methyl-1,3-propanediol (AMPD) as shown for SO1 861 in formula (3):

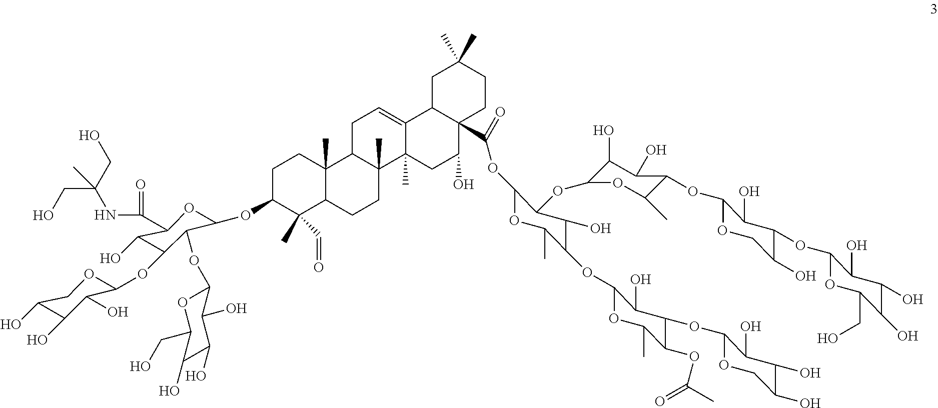

or a saponin molecule having a formula according to one of the following formulas (9)-(12):

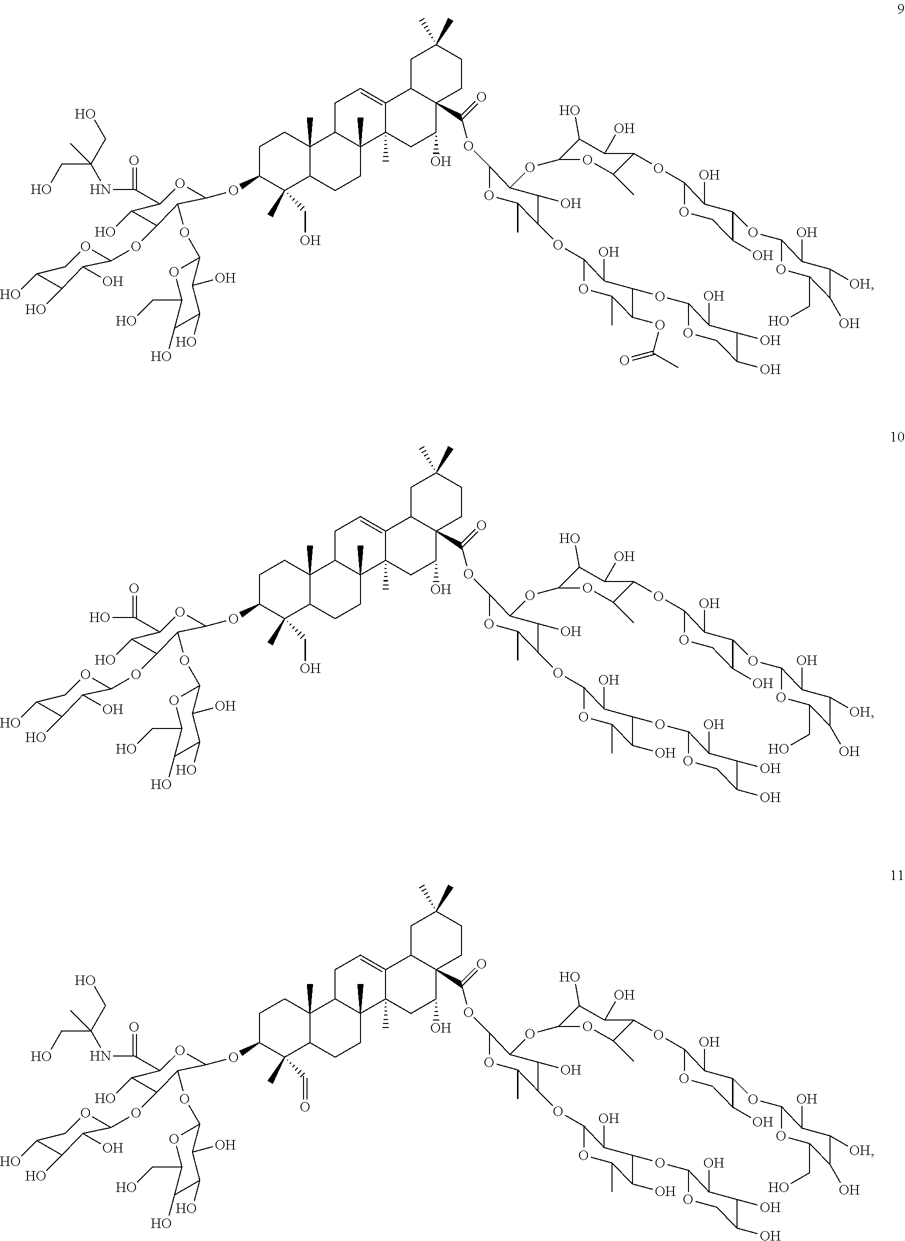

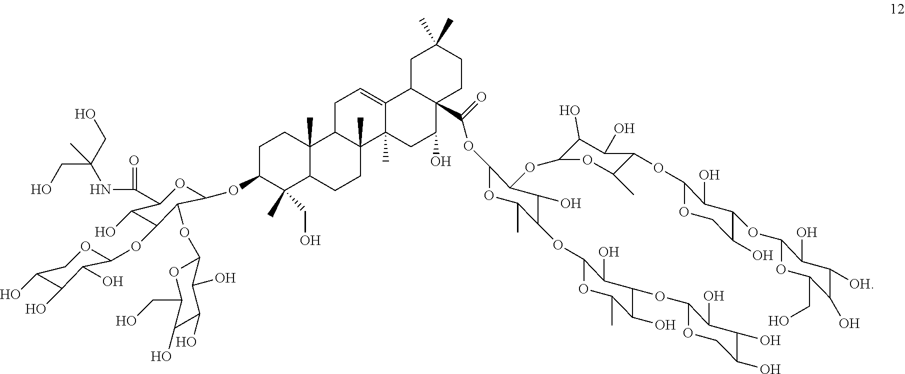

In certain preferred embodiments, the saponin moiety or the saponin is a saponin moiety or saponin with a glucuronic acid group in the carbohydrate substituent at the C-3beta-OH group, preferably selected from the group consisting of (refer to Table 2A for the structural details): NP-017777, NP-017778, NP-017774, NP-018110, NP-017772, NP-018109, NP-017888, NP-017889, NP-018108, SA1641, AE X55, SO1658, gypsoside A, Gypsophila saponin 1 (Gyp1), NP-017674, NP-017810, AG1, NP-003881, NP-017676, NP-017677, NP-017706, NP-017705, NP-017773, NP-017775, SA1657, AG2, GE1741, SO1 542, SO1 584, SO1 674, SO1 700, Saponarioside B, SO1 730, SO1 772, SO1 832 (protonated SO1831; also referred to as Saponarioside A), SO1861 (deprotonated SO1862), SO1862 (protonated SO1861; also referred to as Sapofectosid), S01904, QS-7 (also referred to as QS1861), QS-7 api (also referred to as QS1862), QS-17, QS-18, QS-21 A-apio, QS-21 A-xylo, QS-21 B-apio, QS-21 B-xylo, QS-21, Agrostemmoside E (also referred to as AG1856 or AG2.8), NP-005236, NP-012672, beta-Aescin (described: Aescin Ia), Aescinate, Teaseed saponin I, Teaseedsaponin J, Assamsaponin F, Primula acid 1.

In certain preferred embodiments, the saponin moiety or the saponin is a saponin moiety or saponin that does not comprise an aldehyde function linked to the C-4 atom of the aglycon core structure, preferably selected from the group consisting of (refer to Table 2A for the structural details): NP-005236, AMA-1, AMR, alpha-Hederin, NP-012672, beta-Aescin (described: Aescin Ia), Aescinate, dipsacoside B, esculentoside A, Teaseed saponin I, Teaseedsaponin J, Assamsaponin F, Primula acid 1, AS64R, Macranthoidin A, saikosaponin A, saikosaponin D, AS6.2.

In certain preferred embodiments, the saponin moiety or the saponin is a saponin moiety or saponin with a glucuronic acid group in the carbohydrate substituent at the C-3beta-OH group and that does not comprise an aldehyde function linked to the C-4 atom of the aqlycon core structure, preferably selected from the group consisting of (refer to Table 2A for the structural details): NP-005236, NP-012672, beta-Aescin (described: Aescin la, Aescinate, dipsacoside B, esculentoside A, Teaseed saponin I, Teaseedsaponin J, Assamsaponin F, Primula acid 1, Macranthoidin A, saikosaponin A, saikosaponin D.

When the saponin component comprises the saponin moiety, the saponin moiety is any one of the here-above defined saponin molecules with covalently bound thereto:
a linker, such as a linker suitable for covalently binding the saponin molecule to a further molecule, wherein the linker comprises or is for example:
a. a polyethylene glycol (PEG) with a length of any number between 2 and 60 (PEG2, PEG3, PEG4, PEG5, PEG6, PEG7-PEG10, PEG11-PEG25, PEG25-PEG50, etc.);
b. a peptide;
c. a linear or branched or cyclic alkyl, a linear or branched or cyclic alkenyl, a linear or branched or cyclic alkynyl;
d. a polymeric structure or an oligomeric structure, for example:
wherein the polymeric or oligomeric structure is selected from:
   i. poly- or oligo(amines), such as polyethylenimine and poly(amidoamine),
   ii. polyethylene glycols,
   iii. poly- or oligo(esters), such as poly(lactids),
   iv. poly(lactams),
   v. polylactide-co-glycolide copolymers,
   vi. poly- or oligosaccharides, such as cyclodextrin and polydextrose,
   vii. poly- or oligo(amino acids), such as proteins, peptides and polylysine, and
   viii. DNA oligomers or polymers, RNA polymers, stabilized RNA polymers and PNA (peptide nucleic acid) polymers, and/or
   ix. dendron of type G2, G3, G4 or G5;
a linker, such as a linker as hereabove defined, with a further molecule covalently bound to the linker
wherein said further molecule is any one or more of:
a. a further linker, such as a linker as hereabove defined;
b. a ligand for binding to an endocytic cell-receptor,
   wherein the ligand is a proteinaceous ligand or a non-proteinaceous ligand or a combination thereof,
      I. wherein the non-proteinaceous ligand is for example a ligand for asialoglycoprotein receptor (ASGPR), wherein the ligand for ASGPR comprises at least one N-acetylgalactosamine (GalNAc) moiety, preferably three or four GalNAc moieties, more preferably the ligand for ASGPR comprises three GalNAc moieties, and preferred examples of such non-proteinaceous ligand are:
         tri-GalNAc according to molecule (DD1) or according to molecule (DD2):

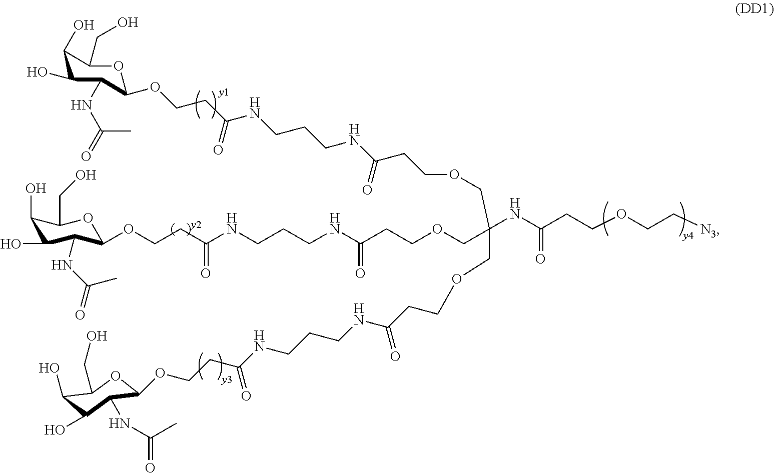

wherein y1, y2 and y3 each are an integer independently selected from 0-20, preferably 1-15, more preferably 2-12, even more preferably 2-10, even more preferably 2-8, most preferably 2 and 3, and preferably y1, y2 and y3 are the same, and y4 is an integer selected from 1-100, preferably 2-80, more preferably 3-70, even more preferably 4-60, even more preferably 4-50, even more preferably 4-40, even more preferably 4-30, even more preferably 4-20, even more preferably 4-6, most preferably 4-5, such as 4.

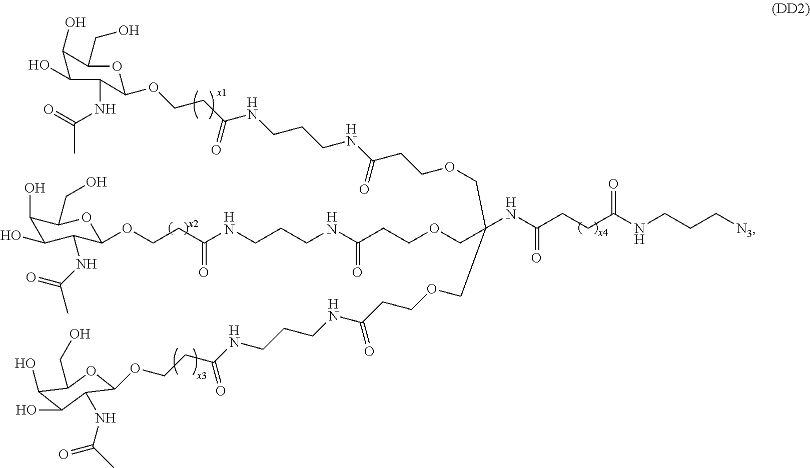

wherein x1, x2 and x3 each are an integer independently selected from 0-20, preferably 1-15, more preferably 2-12, even more preferably 2-10, even more preferably 2-8, most preferably 2 and 3, and preferably x1, x2 and x3 are the same, and x4 is an integer selected from 1-50, preferably 2-40, more preferably 3-30, even more preferably 4-20, even more preferably 5-15, most preferably 8-12, such as 9, preferably tri-GalNAc according to molecule (DD3) or according to molecule (DD4):

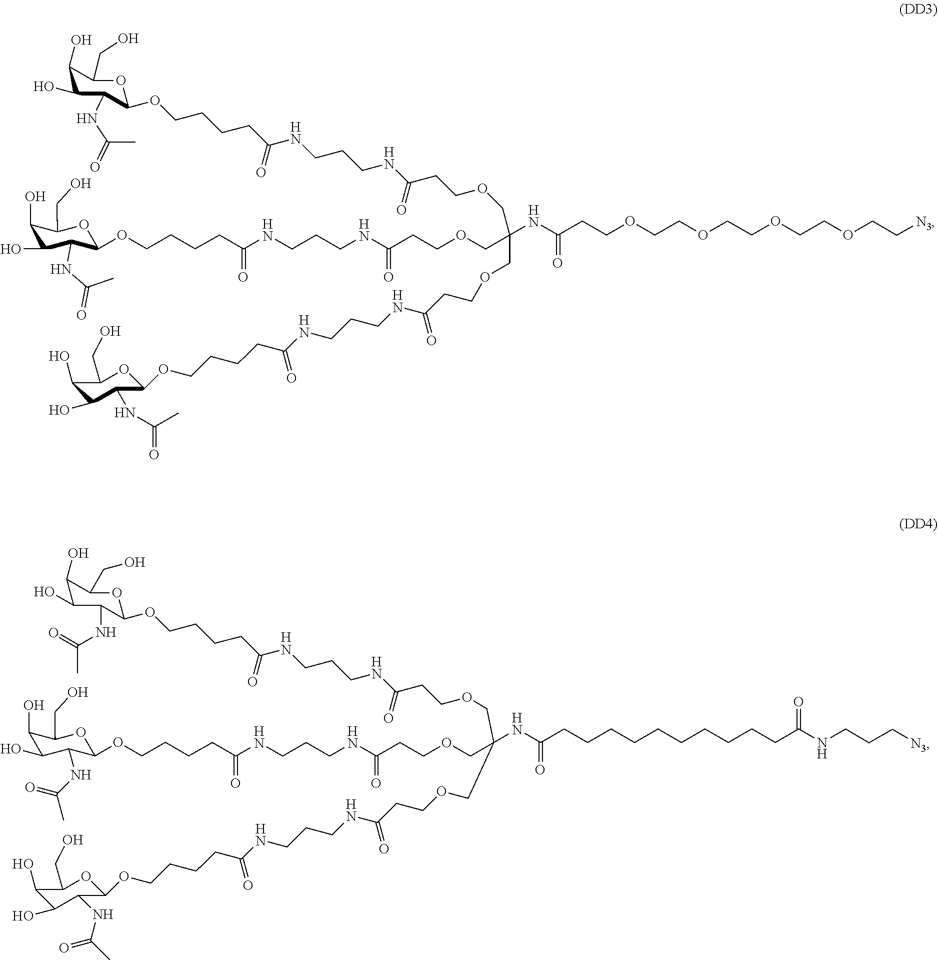

or, the ligand mono-GalNAc represented by Molecule II′:

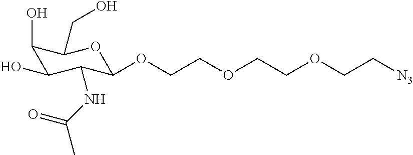 [SMILES: CC(=O)NC1C(OCCOCCOCCN=[N+]=[N-])OC(CO)C(O)C1O]

### (Moledule

and/or
II. wherein the proteinaceous ligand is for example:
a. a protein ligand capable of binding to a(n) endocytic cell-surface receptor, which binding results in internalization of the protein ligand, for example a cytokine or EGF;
b. an antibody, wherein the antibody is defined as an immunoglobulin (Ig) or a functional binding fragment or binding domain thereof.

The saponin component is suitable for passive or active transfer from outside a cell to inside said cell. Moreover, the saponin is suitable for transfer from outside a cell into said cell, being the transfer in the endosomes of said cell. The saponin component is suitable for entry into a cell upon binding of a ligand for binding to an endocytic cell-receptor, bound to the saponin moiety comprised by the saponin component, to said endocytic cell receptor, via endocytosis. Upon binding of the ligand, endocytosis occurs and the saponin component is delivered in the endosomes of the cell bearing the cell receptor.

Examples of such cell-surface receptors are: CD71, CA125, EpCAM(17-1A), CD52, CEA, CD44v6, FAP, EGF-IR, integrin, syndecan-1, vascular integrin alpha-V beta-3, HER2, EGFR, CD20, CD22, Folate receptor 1, CD146, CD56, CD19, CD138, CD27L receptor, prostate specific membrane antigen (PSMA), CanAg, integrin-alphaV, CA6, CD33, mesothelin, Cripto, CD3, CD30, CD239, CD70, CD123, CD352, DLL3, CD25, ephrinA4, MUC-1, Trop2, CEACAM5, CEACAM6, HER3, CD74, PTK7, Notch3, FGF2, C4.4A, FLT3, CD38, FGFR3, CD7, PD-L1, CTLA-4, CD52, PDGFRA, VEGFR1, VEGFR2, c-Met (HGFR), EGFR1, RANKL, ADAMTS5, CD16, CXCR7 (ACKR3), glucocorticoid-induced TNFR-related protein (GITR). Preferred endocytic cell-surface receptors are: HER2, c-Met, VEGFR2, CXCR7, CD71, EGFR and EGFR1.

Ligands for binding to such endocytic cell-surface receptors are for example comprised by the saponin component and/or by the effector component (such as the nucleic acid component) when the effector molecule or effector moiety comprised by the effector component should exert its therapeutic or prophylactic activity in a tumor cell.

Examples of endocytic receptors that can be selected for targeting by a ligand comprised by the saponin component (and/or comprised by the effector component such as the nucleic acid component) are: transferrin receptor (CD71), insulin-like growth factor 1 (IGF-I) receptor (IGF1R), tetraspanin CD63; muscle-specific kinase (MuSK), glucose transporter GLUT4, cation independent mannose 6 phosphate receptor (CI-MPR), and LDL receptor. Ligands for binding to such endocytic cell-surface receptors are for example comprised by the saponin component and/or by the effector component (such as the nucleic acid component) when the effector molecule or effector moiety comprised by the effector component should exert its therapeutic or prophylactic activity in a muscle cell.

An example of an endocytic receptor that can be selected for targeting by a ligand comprised by the saponin component (and/or comprised by the effector component such as the nucleic acid component) is asialoglycoprotein receptor (ASGPR). Ligands for binding to this endocytic cell-surface receptor ASGPR are for example comprised by the saponin component and/or by the effector component (such as the nucleic acid component) when the effector molecule or effector moiety comprised by the effector component should exert its therapeutic or prophylactic activity in the liver, i.e. in a hepatocyte.

When the proteinaceous ligand comprised by the saponin component (and suitable for binding to an endocytic cell-surface receptor) is an antibody, the antibody is for example selected from IgG, IgM, IgE, IgA, or IgD, or any antigen-binding fragment thereof, preferably is selected from a monoclonal antibody, polyclonal antibody, human antibody, humanized antibody, chimeric antibody, resurfaced antibody, anti-idiotypic antibody, mouse antibody, rat antibody, rat/mouse hybrid antibody, llama antibody, llama heavy-chain only antibody, heavy-chain only antibody, a molecule comprising or consisting of a Vhh domain, a Vh domain, a Fab, an scFv, an Fv, a single domain antibody (sdAb), an F(ab)₂, Fcab fragment. A monoclonal antibody and a Fab and a single sdAb or a string of covalently linked sdAb's is preferred.

The linker covalently bound to the saponin molecule, forming the saponin component comprising the saponin moiety and the linker (and in some embodiments a ligand covalently bound to the linker), is in preferred embodiments covalently bound to the saponin via a bond that is cleavable under conditions present in the endosome of mammalian cells, for example human cells. Such cleavable bond is for example subject to cleavage under acidic, reductive, enzymatic and/or light-induced conditions; preferably wherein the cleavable bond is selected from:
a bond subject to cleavage under acidic conditions such as a semicarbazone bond, a hydrazone bond, an imine bond, an acetal bond including a 1,3-dioxolane bond, a ketal bond, an ester bond, and/or an oxime bond,
a bond susceptible to proteolysis, for example amide or peptide bond, preferably subject to proteolysis by Cathepsin B;
a red/ox-cleavable bond such as a disulfide bond, or a thiol-exchange reaction-susceptible bond such as a thio-ether bond
   preferably being an acid-sensitive bond subject to cleavage in vivo under acidic conditions present in endosomes and/or lysosomes of human cells, preferably at pH 4.0-6.5, and more preferably at pH≤5.5; more preferably being an acid-sensitive bond selected from any one or more of: a semicarbazone bond, a hydrazone bond, an imine bond, an acetal bond including a 1,3-dioxolane bond, a ketal bond, an ester bond, and/or an oxime bond, even more preferably selected from a semicarbazone bond and a hydrazone bond; most preferably being a hydrazone bond.

In an embodiment of the invention, the saponin molecule comprises a glucuronic acid function with a carboxylic acid functional group in a carbohydrate substituent at the C-3beta-OH group of the saponin, wherein the carboxylic acid functional group is transformed into an active ester.

In an embodiment of the invention, the saponin moiety comprises a glucuronic acid function with a carboxylic acid functional group in a carbohydrate substituent at the C-3beta-OH group of the saponin, wherein the carboxylic acid functional group is transformed into an active ester upon binding of a linker to said carboxylic acid functional group. In an embodiment, a ligand as hereabove defined is covalently bound to said linker which linker is bound to the saponin moiety. An example of such a saponin moiety comprising an active ester is the moiety resulting from activation of the carboxylic group of the saponin molecule selected for providing the saponin moiety, via 1-[Bis(dimethylamino)methylene]-1H-1,2,3-triazolo[4,5-b]pyridinium 3-oxide hexafluorophosphate (HATU).

In embodiments, the linker that is bound to the saponin molecule in the saponin component further comprises an oligomeric or polymeric structure either being a dendron such as a poly-amidoamine (PAMAM) dendrimer, or a poly-ethylene glycol such as any of PEG3-PEG30; preferably the polymeric or oligomeric structure being any one of PEG4-PEG12 or any one of a G2 dendron, a G3 dendron, a G4 dendron and a G5 dendron, more preferably being a G2 dendron or a G3 dendron or a PEG3-PEG30.

For example, the saponin component comprises a saponin moiety comprising a covalently bound linker and is a molecule according to any one of formula (I)-(V):

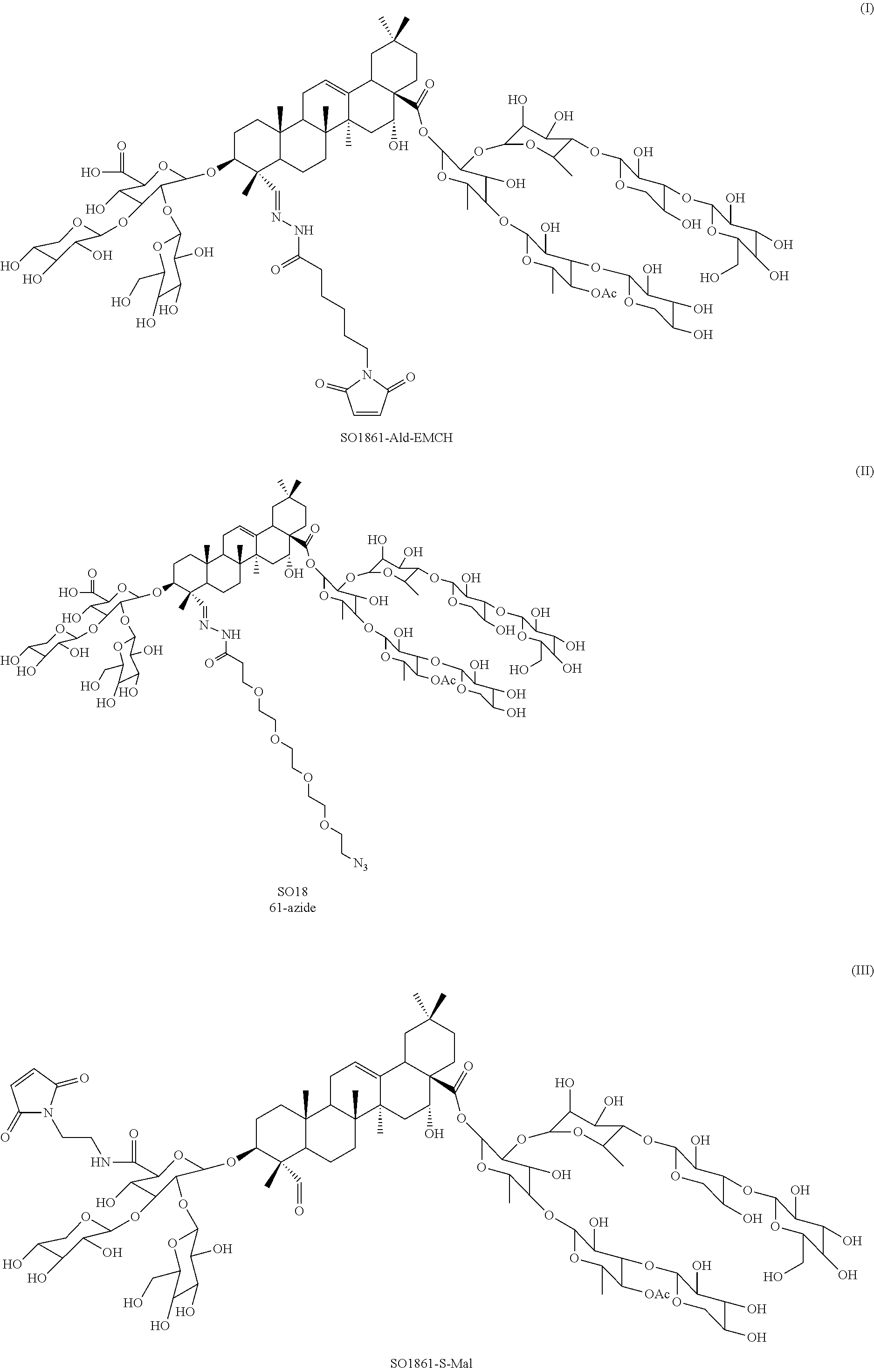

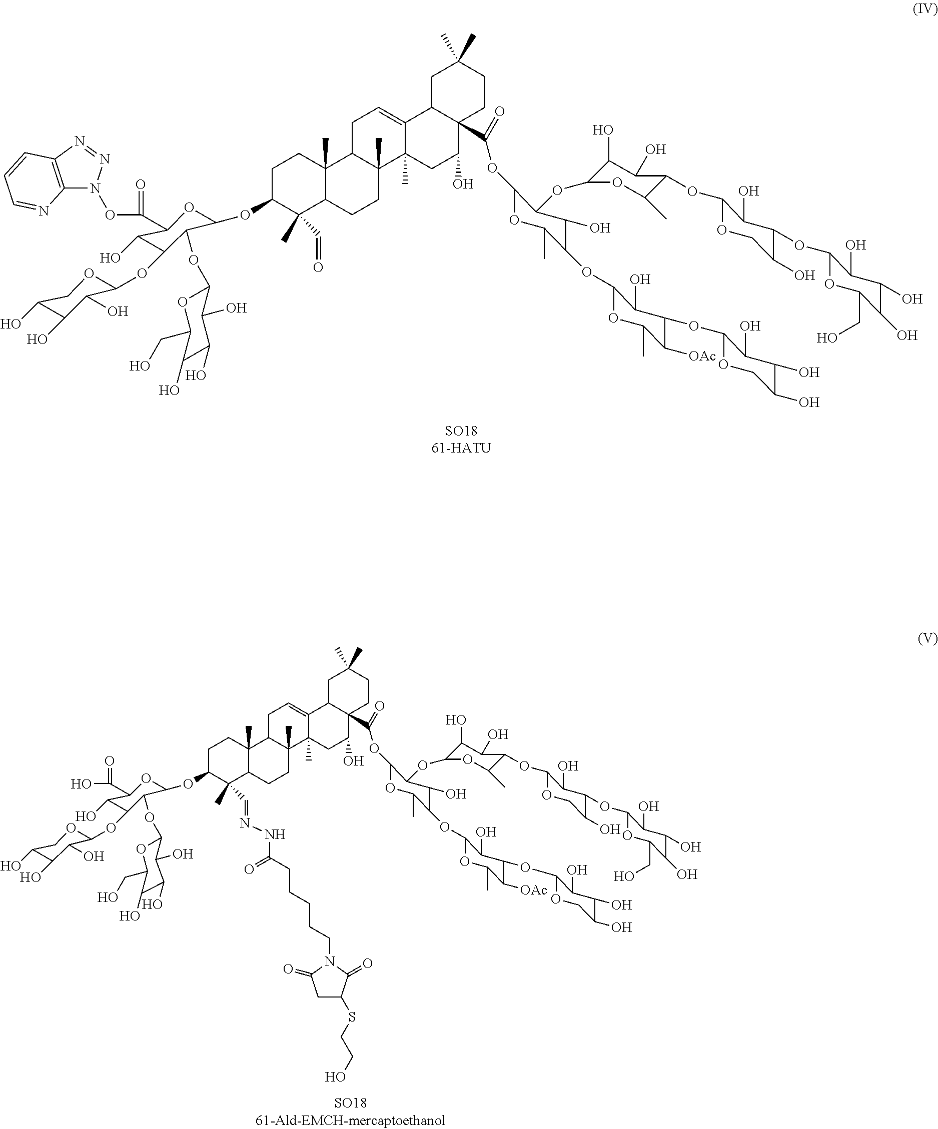

and/or for example the saponin component comprises
a saponin, wherein the carboxyl group of the glucuronic acid unit in the first saccharide chain bound to C-3 of the aglycone core structure of the saponin is transformed into an amide bond through reaction with N-(2-aminoethyl)maleimide (AEM) as shown for SO1861 in formula (18):

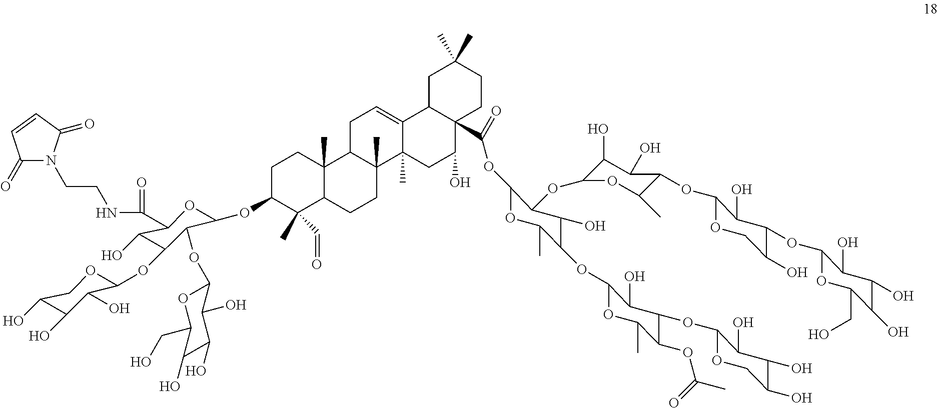

or a saponin having a formula according to one of the following formulas (14)-(16) and (19)-(21):

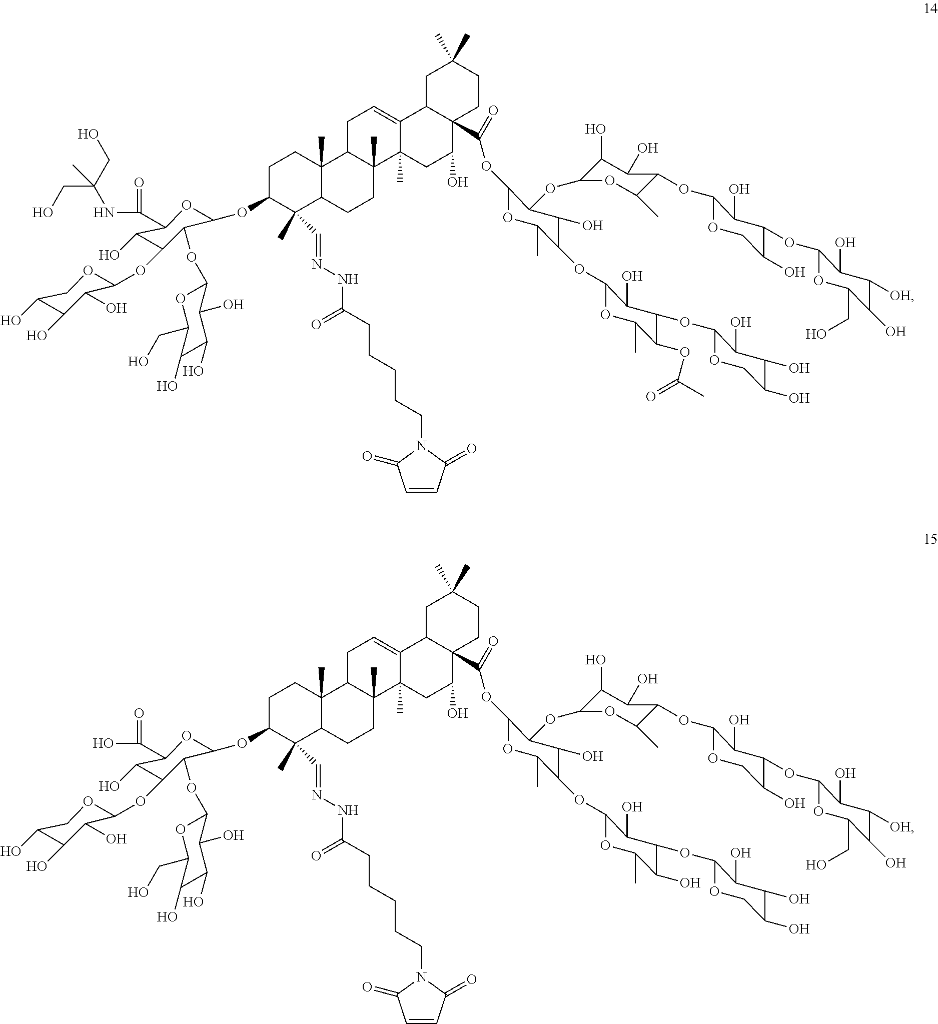

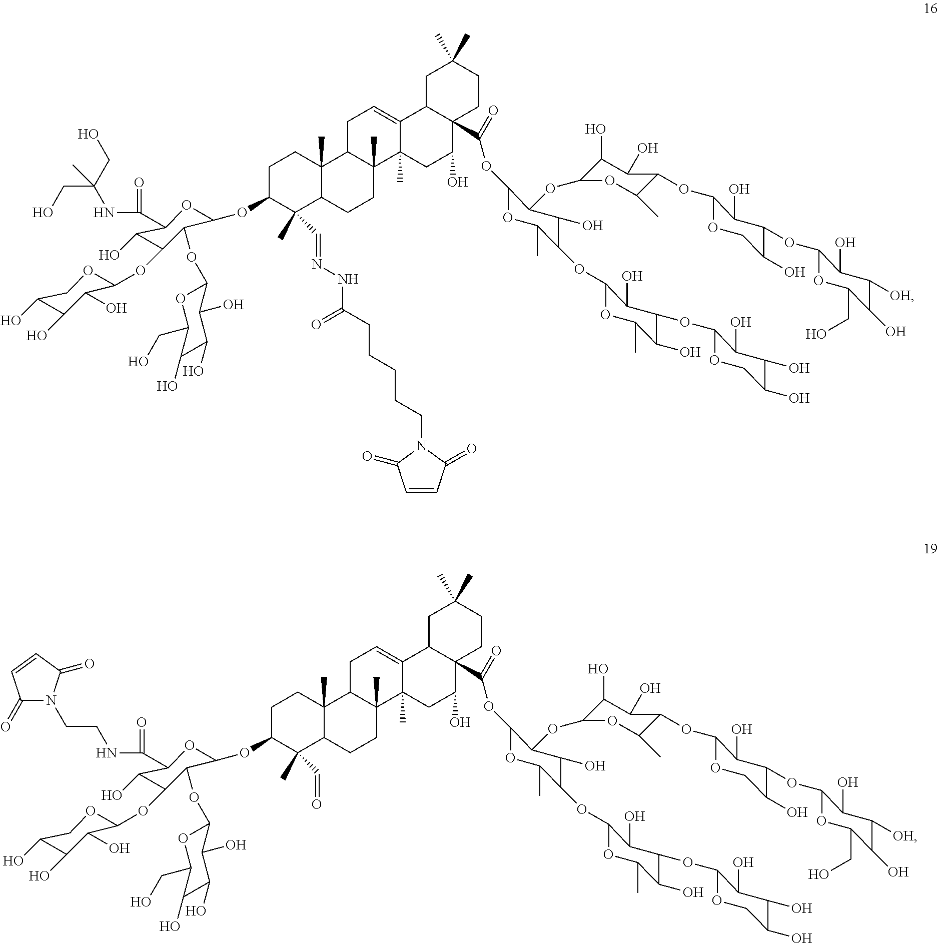

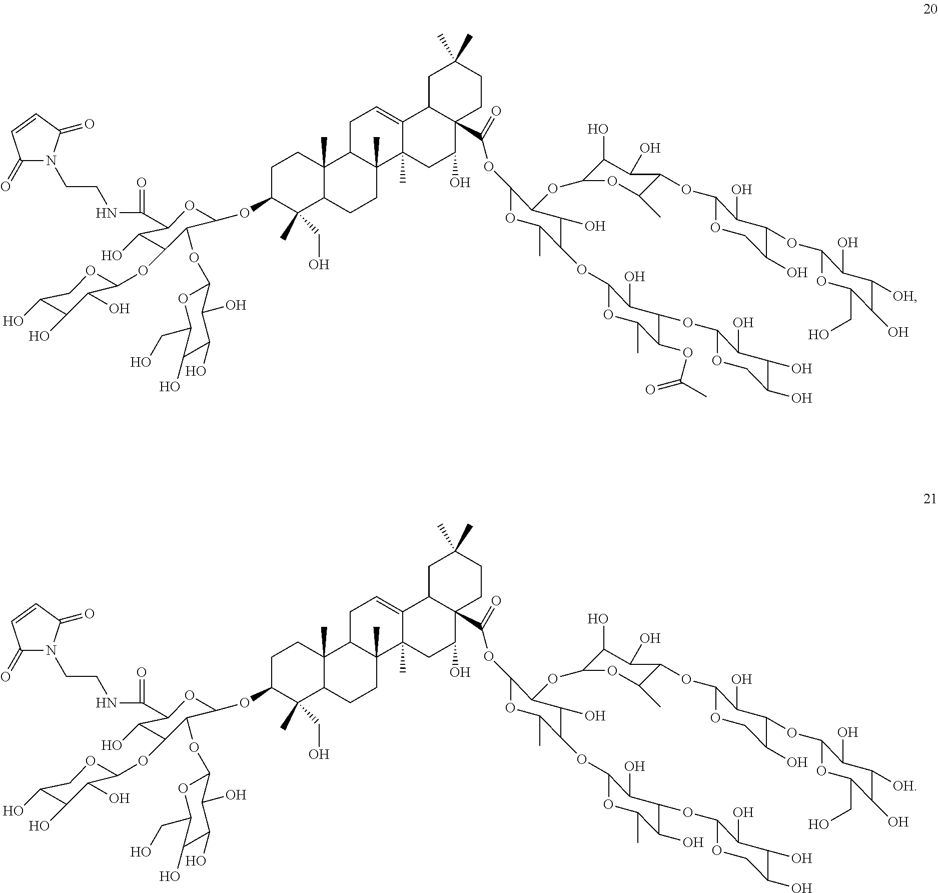

In a preferred embodiment, the saponin component is the molecule according to formula (I) here above or is SO1861 or is a conjugate of SO1861 and one or more GalNAc moieties, preferably three GalNAc moieties.

### The Effector Component

As explained herein before, the term “effector component”, in its broadest sense, refers to a component comprising an effector moiety or consisting of an effector molecule, wherein the effector moiety or the effector molecule is:
a proteinaceous molecule;
a non-proteinaceous molecule:
a combination of a proteinaceous molecule and a non-proteinaceous molecule (for example, a moiety or molecule comprising a peptide and a nucleic acid or lipid, or a nucleic acid and a lipid or a G2, G3 or G4 dendron such as PAMAM).

In an embodiment, the effector component is or comprises a toxin such as a proteinaceous toxin.

In an embodiment, the effector component is or comprises an enzyme such as urease or Cre-recombinase.

In an embodiment, the effector component is or comprises a toxin, wherein the toxin is selected from the list consisting of: a viral toxin, a bacterial toxin, a plant toxin including ribosome-inactivating proteins and the A chain of type 2 ribosome-inactivating proteins, an animal toxin, a human toxin and a fungal toxin, more preferably the toxin is a plant toxin including ribosome-inactivating proteins and the A chain of type 2 ribosome-inactivating proteins.

In an embodiment, the effector component is or comprises a toxin, wherein the toxin is selected from the list consisting of: apoptin, Shiga toxin, Shiga-like toxin, Pseudomonas aeruginosa exotoxin (PE), full-length or truncated diphtheria toxin (DT), cholera toxin, alpha-sarcin, dianthin, saporin, bouganin, de-immunized derivative debouganin of bouganin, shiga-like toxin A, pokeweed antiviral protein, ricin, ricin A chain, modeccin, modeccin A chain, abrin, abrin A chain, volkensin, volkensin A chain, viscumin, viscumin A chain, frog RNase, granzyme B, human angiogenin; preferably the toxin is dianthin and/or saporin.

In an embodiment, the effector component is or comprises a toxin, wherein the toxin is selected from the list consisting of: a toxin targeting ribosomes, a toxin targeting elongation factors, a toxin targeting tubulin, a toxin targeting DNA and a toxin targeting RNA, more preferably the toxin is selected from the list consisting of: emtansine, pasudotox, maytansinoid derivative DM1, maytansinoid derivative DM4, monomethyl auristatin E (MMAE, vedotin), monomethyl auristatin F (MMAF, mafodotin), a Calicheamicin, N-Acetyl-γ-calicheamicin, a pyrrolobenzodiazepine (PBD) dimer, a benzodiazepine, a CC-1065 analogue, a duocarmycin, Doxorubicin, paclitaxel, docetaxel, cisplatin, cyclophosphamide, etoposide, docetaxel, 5-fluorouracyl (5-FU), mitoxantrone, a tubulysin, an indolinobenzodiazepine, AZ13599185, a cryptophycin, rhizoxin, methotrexate, an anthracycline, a camptothecin analogue, SN-38, DX-8951f, exatecan mesylate, truncated form of Pseudomonas aeruginosa exotoxin (PE38), a duocarmycin derivative, an amanitin, α-amanitin, a spliceostatin, a thailanstatin, ozogamicin, tesirine, Amberstatin269 and soravtansine.

In an embodiment, the effector component is or comprises a small molecule therapeutic. Preferably, the small molecule therapeutic has a molecular weight of 1200 Dalton (Da) or less, preferably less than 1000 Da, preferably less than 800 Da, more preferably less than 600 Da.

In particularly preferred embodiments of the invention, the effector component is or comprises a nucleic acid or oligonucleotide therapeutic, wherein the nucleic acid or oligonucleotide is selected from deoxyribonucleic acid (DNA) oligomer, ribonucleic acid (RNA) oligomer, antisense oligonucleotide (ASO, AON), short interfering RNA (siRNA), anti-microRNA (anti-miRNA), DNA aptamer, RNA aptamer, mRNA, mini-circle DNA, peptide nucleic acid (PNA), phosphoramidate morpholino oligomer (PMO), phosphorothioate-modified antisense oligonucleotide (PS-ASO), 2′-O-methyl (2′-OMe) phosphorothioate RNA, 2′-O-methoxyethyl (2′-O-MOE) RNA {2′-O-methoxyethyl-RNA (MOE)}, locked nucleic acid (LNA), bridged nucleic acid (BNA), 2′-deoxy-2′-fluoroarabino nucleic acid (FANA), 2′-O-methoxyethyl-RNA (MOE), 3′-fluoro hexitol nucleic acid (FHNA), glycol nucleic acid (GNA), xeno nucleic acid oligonucleotide and threose nucleic acid (TNA). For example, the nucleic acid is a BNA for silencing HSP27 protein expression or a BNA for silencing apolipoprotein B expression, or is an ASO for silencing STAT3 expression, or a PMO for excluding (skipping) an exon on the pre-mRNA to result in a shortened protein.

In an embodiment of the invention, the effector component is or comprises a nucleic acid, wherein the nucleic acid is selected from any one or more of a(n): short interfering RNA (siRNA), short hairpin RNA (shRNA), anti-hairpin-shaped microRNA (miRNA), single-stranded RNA, aptamer RNA, double-stranded RNA (dsRNA), anti-microRNA (anti-miRNA, anti-miR), antisense oligonucleotide (ASO), mRNA, DNA, antisense DNA, locked nucleic acid (LNA), bridged nucleic acid (BNA), 2′-0,4′-aminoethylene bridged nucleic Acid (BNA^{NC}), BNA-based siRNA, and BNA-based antisense oligonucleotide (BNA-AON).

In an embodiment of the invention, the effector component is or comprises a nucleic acid, wherein the nucleic acid is selected from any one of an anti-miRNA, a BNA-AON or an siRNA, such as BNA-based siRNA, preferably selected from chemically modified siRNA, metabolically stable siRNA and chemically modified, metabolically stable siRNA.

In an embodiment of the invention, the effector component is or comprises a nucleic acid, wherein the nucleic acid is an oligonucleotide that is capable of silencing a gene, when present in a cell comprising such gene, wherein the gene is for example any one of genes: dystrophin, STAT3a, apolipoprotein B (apoB), HSP27, transthyretin (TTR), proprotein convertase subtilisin/kexin type 9 (PCSK9), delta-aminolevulinate synthase 1 (ALAS1), antithrombin 3 (AT3), glycolate oxidase (GO), complement component C5 (CC5), X gene of hepatitis B virus (HBV), S gene of HBV, alpha-1 antitrypsin (AAT) and lactate dehydrogenase (LDH), and/or is capable of targeting an aberrant miRNA when present in a cell comprising such aberrant miRNA.

In a preferred embodiment of the invention, the effector component is or comprises a nucleic acid, wherein the nucleic acid is an oligonucleotide that is capable of silencing a gene, when present in a cell comprising such gene, wherein the gene is SERPINC.

In an embodiment of the invention, the effector component is or comprises a nucleic acid, wherein the nucleic acid is an oligonucleotide that is capable of targeting an mRNA, when present in a cell comprising such mRNA, wherein for example the mRNA is involved in expression of any one of proteins: dystrophin, STAT3a, apoB, HSP27, TTR, PCSK9, ALAS1, AT3, GO, CC5, expression product of X gene of HBV, expression product of S gene of HBV, AAT and LDH, or is for example capable of antagonizing or restoring an miRNA function such as inhibiting an oncogenic miRNA (onco-miR) or suppressing of expression of an onco-miR, when present in a cell comprising such an miRNA.

In an embodiment of the invention. the nucleic acid comprised by the nucleic acid component is defined as a nucleic acid that is no longer than 150 nt, preferably wherein the oligonucleotide has a size of 5-150 nt, preferably being 8-100 nt, most preferably being 10-50 nt.

In an embodiment of the invention, the nucleic acid comprised by the nucleic acid component is an antisense oligonucleotide, preferably being a mutation specific antisense oligonucleotide, most preferably being an oligonucleotide designed to induce exon skipping. Preferably, the nucleic acid comprised by the nucleic acid component comprises or consists of a morpholino phosphorodiamidate oligomer (PMO) or a 2′-O-methyl (2′-OMe) phosphorothioate RNA or a 2MOE (2′-O-(2-Methoxyethyl)-oligoribonucleotides (2′-O-MOE bases)).

In a preferred embodiment of the invention, the effector component is or comprises a nucleic acid, wherein the nucleic acid targets a gene selected from the group consisting of IRS1, ICAM1, TTR, FUS, APOC3, LPA, CEP290, SOD1, HTT, TGFB2, GFAP, CCR3/CSF2RB, GHR, ITGA4, PCSK9, FOXP3, viral HBV, viral UL123, ApoB100, ARSA, ALAS, GO, VEGF, MAPT, PCED, STAT3, KLB1, DYN2, UBE2A, DGAT2, SNCA, ATXN2, LRRK2, AGT, F11, GCGR, KLBK1, AR, SCNNIA, TMPRSS6, TGFB2, DMD (dystrophin), GRB2, RHO, USH2A, SCN1A, ANGPTL3, C2orf72, SERPINC1, LDHA, CASP2, TP53, TRPV1, SERPINA1, HSD17B13, ANGPTL3, APOC3, ADRB2, SERPINA1, SERPINHI, C5, CHST15, CTGF, KRAS, PTGS2/TGFB1, HBsAG, MIR21, CEBPA, MIR29B1, ALAS1, HAO1, SMN2, APOb, CMV virus IE2, GJA1 and CFB.

In a preferred embodiment of the invention, the effector component is or comprises a nucleic acid, wherein the nucleic acid targets the gene SERPINC1.

In a preferred embodiment of the invention, the effector component is or comprises a nucleic acid or oligonucleotide therapeutic, wherein the nucleic acid or oligonucleotide therapeutic is selected from the group consisting of fomivirsen, mipomersen, nusinersen, eteplirsen, golodirsen, viltolarsen, casimersen, defibrotide, inotersen, patisiran, vutrisiran, givosiran, lumasiran, inclisiran, pegaptanib, volanesorsen, aganirsen, alicaforsen, eplontersen, ION-363, olezarsen, pelacarsen, sepofarsen, tofersen, tominersen, trabedersen, zilganersen, ASM-8, atesidorsen, ATL-1102, AZD-8233, AZD-8701, bepirovirsen, B1II1B-080, cepadacursen, cimderlirsen, CODA-001, danvatirsen, donidalorsen, DYN-101, GTX-102, ION-224, ION-253, ION-464, ION-541, ION-859, IONIS-AGTLRx, IONIS-FB-LRx, IONIS-FXILRx, IONIS-GCGRRx, IONIS-HBVLRx, IONIS-PKKRx, IONISAR-2.5Rx, IONISENAC-2.5Rx, IONISTMPRSS-6LRx, ISTH-0036, NS-089, prexigebersen, QR-1123, QR-421a, renadirsen, SRP-5051, STK-001, vupanorsen, WVE-003, WVE-004, WVEN-531, fitusiran, nedosiran, QPI-1007, teprasiran, tivanisiran, AB-729,ALNAAT-02, ARO-HSD, AROANG-3, AROAPOC-3, bamosiran, belcesiran, BMS-986263, cemdisiran, fazirsiran, JNJ-3989, MT-5745, olpasiran, OLX-101, RG-6346, siG-12D-LODER, SR-063, STP-705, VIR-2218, zilebesiran, lademirsen, MTL-CEBPA, remlarsen, and therapeutically equivalent variants of any of these nucleic acid or oligonucleotide therapeutics.

In a preferred embodiment of the invention, the effector component is or comprises a nucleic acid or oligonucleotide therapeutic, optionally comprising one or more GalNAc moieties covalently bound thereto, and preferably selected from the group consisting of: fomivirsen, mipomersen, nusinersen, eteplirsen, golodirsen, viltolarsen, casimersen, Defibrotide, inotersen, patisiran, vutrisiran (comprising conjugated GalNAc), givosiran (comprising conjugated GalNAc), lumasiran (comprising conjugated GalNAc), inclisiran (comprising conjugated GalNAc), pegaptanib, volanesorsen, aAganirsen, alicaforsen, plontersen (comprising conjugated GalNAc), ION-63, olezarsen (comprising conjugated GalNAc), pelacarsen (comprising conjugated GalNAc), sepofarsen, tofersen, tominersen, trabedersen, zilganersen, ASM-8, atesidorsen, ATL-1102, AZD-8233 (comprising conjugated GalNAc), AZD-8701, bepirovirsen, GSK 3389404 (comprising conjugated GalNAc), B1IIB-080, cepadacursen, cimderlirsen, CODA-001, danvatirsen, donidalorsen (comprising conjugated GalNAc), DYN-101, GTX-102, ION-224 v (comprising conjugated GalNAc), ION-253, ION-464, ION-541, ION-859, IONIS-AGTLRx (comprising conjugated GalNAc), IONIS-FB-LRx (comprising conjugated GalNAc), IONIS-FXILRx (comprising conjugated GalNAc), IONIS-GCGRRx, IONIS-HBVLRx (comprising conjugated GalNAc), IONIS-PKKRx (comprising conjugated GalNAc), IONISAR-2.5Rx, IONISENAC-2.5Rx, IONISTMPRSS-6LRx (comprising conjugated GalNAc), ISTH-0036, NS-089, prexigebersen, QR-1123, QR-421a (ultevursen), renadirsen, SRP-5051, STK-001, vupanorsen (comprising conjugated GalNAc), WVE-003, WVE-004, WVEN-531, fitusiran (comprising conjugated GalNAc), nedosiran (comprising conjugated GalNAc), QPI-1007, teprasiran, tivanisiran, AB-729 (comprising conjugated GalNAc), ALNAAT-02 (comprising conjugated GalNAc), ARO-HSD, AROANG-3 (comprising conjugated GalNAc), AROAPOC-3 (comprising conjugated GalNAc), bamosiran, belcesiran (GaIXC; uses tetra-anternnary GalNAc, instead of tri-antennary GalNAc), BMS-986263, cemdisiran (comprising conjugated GalNAc), fazirsiran (comprising conjugated GalNAc), JNJ-3989 (comprising conjugated GalNAc), MT-5745, olpasiran (comprising conjugated GalNAc), OLX-101, RG-6346 (comprising conjugated GalNAc), siG-12D-LODER, SR-063, STP-705, VIR-2218 (comprising conjugated GalNAc), Zilebesiran (comprising conjugated GalNAc), lademirsen, MTL-CEBPA, remlarsen.

In a preferred embodiment of the invention, the effector component is or comprises a nucleic acid or oligonucleotide therapeutic, comprising one or more GalNAc moieties covalently bound thereto, and preferably selected from the group consisting of: vutrisiran, givosiran, lumasiran, inclisiran, plontersen, olezarsen, pelacarsen, AZD-8233, GSK 3389404, donidalorsen, ION-224, IONIS-AGTLRx, IONIS-FB-LRx, IONIS-FXILRx, IONIS-HBVLRx, IONIS-PKKRx, IONISTMPRSS-6LRx, vupanorsen, fitusiran, nedosiran, AB-729, ALNAAT-02, AROANG-3, AROAPOC-3, belcesiran, cemdisiran, fazirsiran, JNJ-3989, olpasiran, RG-6346, VIR-2218, Zilebesiran.

In a preferred embodiment of the invention, the effector component is or comprises a nucleic acid or oligonucleotide therapeutic, wherein the nucleic acid or oligonucleotide therapeutic is an siRNA, either or not comprising one or more GalNAc moieties covalently bound thereto, and preferably selected from the group consisting of: fitusiran (comprising conjugated GalNAc), nedosiran (comprising conjugated GalNAc), QPI-1007, teprasiran, tivanisiran, AB-729 (comprising conjugated GalNAc), ALNAAT-02 (comprising conjugated GalNAc), ARO-HSD, AROANG-3 (comprising conjugated GalNAc), AROAPOC-3 (comprising conjugated GalNAc), bamosiran, belcesiran (GalXC; with tetra-antennary GalNAc, instead of tri-antennary GalNAc), BMS-986263, cemdisiran (comprising conjugated GalNAc), fazirsiran (comprising conjugated GalNAc), JNJ-3989 (comprising conjugated GalNAc), MT-5745, olpasiran (comprising conjugated GalNAc), OLX-101, RG-6346 (comprising conjugated GalNAc), siG-12D-LODER, SR-063, STP-705, VIR-2218 (comprising conjugated GalNAc), Zilebesiran (comprising conjugated GalNAc).

In a preferred embodiment of the invention, the effector component is or comprises a nucleic acid or oligonucleotide therapeutic, wherein the nucleic acid or oligonucleotide therapeutic is an siRNA, comprising one or more GalNAc moieties covalently bound thereto, and preferably selected from the group consisting of: fitusiran, nedosiran, AB-729, ALNAAT-02, AROANG-3, AROAPOC-3, belcesiran, cemdisiran, fazirsiran, JNJ-3989, olpasiran, RG-6346, VIR-2218, zlebesiran.

In a preferred embodiment of the invention, the effector component is or comprises a nucleic acid, wherein the nucleic acid or oligonucleotide therapeutic is selected from the group consisting of fomivirsen, mipomersen, nusinersen, eteplirsen, golodirsen, viltolarsen, casimersen, defibrotide, inotersen, patisiran, vutrisiran, givosiran, lumasiran, inclisiran, pegaptanib and volanesorsen.

In embodiments of the invention, the effector moiety is any one of the here-above defined effector molecules, with covalently bound thereto:
a linker selected from the any one or more linkers hereabove defined for the saponin moiety;
a linker, such as a linker as hereabove defined, with a further molecule covalently bound to the linker
wherein said further molecule is defined as hereabove defined for the saponin moiety, and is any one or more of:
c. a further linker, such as a linker as hereabove defined;
d. a ligand for binding to an endocytic cell-receptor, wherein the ligand is a proteinaceous ligand or a non-proteinaceous ligand or a combination thereof,

I. wherein the non-proteinaceous ligand is for example a ligand for asialoglycoprotein receptor
(ASGPR), preferably one or more GalNAc moieties, more preferably three GalNAc moieties as defined for the saponin moiety
   and/or

II. wherein the proteinaceous ligand is for example:
a. a protein ligand capable of binding to a cell-surface receptor, which binding results in internalization of the protein ligand, for example a cytokine or EGF;
b. an antibody, as defined hereabove for the saponin moiety.

In embodiments of the invention wherein the effector component comprises an effector moiety conjugated with a ligand for binding to an endocytic cell-surface receptor, the effector component either comprises the same ligand as the saponin component, or the effector component comprises a ligand that differs from the ligand comprised by the saponin component. When the ligands comprised by the effector component and the saponin component differ, those different ligands typically both bind to an endocytic cell-surface receptor present on the same cell. Such endocytic receptor can be the same endocytic receptor or can be two different endocytic receptors. For example, the same ligand comprised by the effector component and comprised by the saponin component can be one or more GalNAc molecules, preferably three GalNAc molecules. For example, the ligand comprised by the effector component can be an antibody capable of binding to a first tumor-cell specific receptor present on a tumor cell, and the ligand comprised by the saponin component can be an antibody or a ligand such as EGF capable of binding to a second tumor-cell specific receptor present on said same tumor cell.

In accordance with preferred embodiments of the invention, the saponin component comprises a ligand comprising one or more GalNAc moieties, preferably three GalNAc moieties, and the effector component comprises a nucleic acid and a ligand comprising one or more GalNAc moieties, preferably three GalNAc moieties. In a preferred embodiment, the saponin component comprises a ligand comprising one or more GalNAc moieties, preferably three GalNAc moieties, and the nucleic acid component comprises a ligand comprising one or more GalNAc moieties, preferably three GalNAc moieties.

### Therapeutic Indications

Those skilled in the art will understand, based on the present teachings, that the general concepts of the invention will find applicability in a myriad of therapeutic areas or indications. Stated generally, the invention will find applicability, and provide benefits, in any therapy involving the administration of an effector moiety that requires cellular uptake to become effective and/or an effector moiety that acts via an intracellular (molecular) target. More particularly, the invention will find applicability, and provide benefits, in any therapy involving the administration of an effector moiety that requires cellular uptake to become effective and/or an effector moiety that acts via an intracellular (molecular) target, and wherein such effector moiety enters the (target) cell via the endosomes.

Hence, the invention provides methods of treatment as defined herein, wherein the disease or condition can be any one disease or condition that is selected from the group consisting of: cytomegalovirus retinitis (in immunocompromised patients); Homozygous familial hypercholesterolemia and/or (other) apoB-100-related diseases; Spinal muscular atrophy and/or (other) SMN2-related diseases; Duchenne muscular dystrophy and/or (other) DMD-related diseases; Veno-occlusive disease and/or (other) ARSA-related diseases; Hereditary transthyretin-mediated amyloidosis and/or (other) TTR-related diseases; Acute hepatic Porphyria and/or (other) ALAS1-related diseases; Primary hyperoxaluria type 1 and/or (other) GO-related diseases; Primary hypercholesterolemia and/or (other) PCSK9-related diseases; Neovascular (Wet) Age-Related Macular Degeneration and/or (other) VEGF-related diseases; Familial chylomicronemia syndrome; and/or (other) apoC3-related diseases; ocular neovascularization and/or (other) IRS1-related diseases; (acute disease flares in) moderate to severe Inflammatory Bowel Disease (IBD), and/or (other) ICAM1-related diseases; chronic heart failure and high blood pressure, particularly for patients with resistant hypertension due to elevated aldosterone, and/or (other) TTR-related diseases; fused in sarcoma (FUS)-protein associated myotrophic lateral sclerosis (ALS), and/or (other) FUS-related diseases; hypertriglyceridemia, and/or (other) APOC3-related diseases; Elevated Lp(a)-associated diseases, including cardiovascular disease (CVD), and/or (other) LPA-related diseases; CEP290-mediated Leber congenital amaurosis 10 (LCA10), and/or (other) CEP290-related diseases; amyotrophic lateral sclerosis (ALS), and/or (other) SOD1-related diseases; Huntington's disease (HD), and/or (other) HTT-related diseases; Cancers associated with TGFB2-overexpression, including brain cancer, colorectal cancer, melanoma and pancreatic cancer, and/or (other) TGFB2-related diseases; Alexander's Disease (AxD), and/or (other) GFAP-related diseases; moderate-to-severe asthma, and/or (other) CCR3- or CSF2RB-related diseases; excessive growth hormone (GH)-associated diseases, including acromegaly, and/or (other) GHR-related diseases; Duchenne muscular dystrophy (DMD), and/or (other) ITGA4-related diseases; dyslipidemia, and/or (other) PCSK9-related diseases; cancer associated with advanced solid tumours, including clear cell renal cell cancer (ccRCC), non-small-cell lung cancer (NSCLC), triple negative breast neoplasms (TNBN), squamous cell cancer of head and neck (HNSCC), small cell lung cancer (SCLC), gastroesophageal cancer, melanoma, cervical cancer, and/or (other) FOXP3-related diseases; chronic hepatitis B (CHB), and/or (other) viral HBV-related diseases; Alzheimer's Disease, and/or (other) MAPT-related diseases; hypercholesterolaemia, and/or (other) PCSK9-related diseases; Acromegaly, and/or (other) GHR-related diseases; persistent Corneal Epithelial Defects (PCED), and/or (other) GJA1-related diseases; Cancers involving STAT, including lymphoma, lung cancer and head and neck squamous cell carcinoma (HNSCC), and/or (other) STAT3-related diseases; hereditary angioedema (HAE), and/or (other) KLKB1-related diseases; congenital structural myopathies, and/or (other) DYN2-related diseases; Angelman syndrome (AS), and/or (other) UBE2A-related diseases; non-alcoholic steatohepatitis (NASH), and/or (other) DGAT2-related diseases; gastrointestinal autoimmune diseases; Parkinson's disease (PD), multiple system atrophy (MSA) and related synucleinopathies, and/or (other) SNCA-related diseases; amyotrophic lateral sclerosis (ALS), and/or (other) ATXN2-related diseases; Parkinson's disease (PD), and/or (other) LRRK2-related diseases; hypertension and/or chronic heart failure, and/or (other) AGT-related diseases; complement-mediated diseases, including IgA nephropathy and Age-related macular degeneration (AMD); clotting disorders, including thrombosis, and end-stage renal disease (ESRD), and/or (other) F11-related diseases; type 2 Diabetes, and/or (other) GCGR-related diseases; hepatitis B virus (HBV) infections, and/or (other) viral HBV-related diseases; hereditary angioedema (HAE), and/or (other) KLKB1-related diseases; prostate cancer, and/or (other) AR-related diseases; cystic fibrosis, and/or (other) SCNNIA-related diseases; beta-thalassemia, and/or (other) TMPRSS6-related diseases; primary open-angle glaucoma (POAG), and/or (other) TGFB2-related diseases; RAS-activated cancers, including chronic myeloid leukaemia (CML), acute myeloid leukemia (AML), acute lymphocytic leukemia (ALL) and myelodysplastic syndromes (MDS), and/or (other) GRB2-related diseases; retinitis pigmentosa (RP), and/or (other) RHO-related diseases; retinitis pigmentosa (RP), and/or (other) USH2A-related diseases; dravet syndrome, and/or (other) SCN1A-related diseases; diabetes, hepatic steatosis, and hypertriglyceridaemia, and/or (other) ANGPTL3-related diseases; cardiovascular diseases, and/or (other) HTT-related diseases; amyotrophic lateral sclerosis (ALS) and frontotemporal disorders (FTD), and/or (other) C9orf72-related diseases; hemophilia A or B, and/or (other) SERPINC1-related diseases; primary hyperoxaluria (PH), and/or (other) LDHA-related diseases; optic neuropathies including glaucoma, and/or (other) CASP2-related diseases; optic neuropathies including glaucoma, and/or (other) TP53-related diseases; dry eye disease, and/or (other) TRPV1-related diseases; hepatitis B virus (HBV) infections, and/or (other) HBsAg-related diseases; gastrointestinal and metabolic disorders, and/or (other) SERPINA1-related diseases; gastrointestinal disorders including non-alcoholic steatohepatitis, and/or (other) HSD17B13-related diseases; homozygous familial hypercholesterolemia, and/or (other) ANGPTL3-related diseases; familial chylomicronemia syndrome (FCS), and/or (other) APOC3-related diseases; glaucoma and ocular hypertension, and/or (other) ADRB2-related diseases; alpha-1 antitrypsin (AAT) deficiency-associated liver disease (AATLD), and/or (other) SERPINA1-related diseases; advanced hepatic fibrosis, and/or (other) SERPINHI-related diseases; immunoglobulin A nephropathy, and/or (other) C5-related diseases; alpha-1 antitrypsin (AAT) deficiency-associated liver disease (AATLD), and/or (other) SERPINA1-related diseases; hepatitis B virus (HBV) infections, and/or (other) viral HBV-related diseases; ulcerative colitis (=chronic inflammatory bowel disease (IBD), and/or (other) CHST15-related diseases; atherosclerotic cardiovascular diseases, and/or (other) LPA-related diseases; hypertrophic and keloid scars, and/or (other) CTGF-related diseases; chronic hepatitis B virus (HBV) infection, and/or (other) HBsAg-related diseases; pancreatic cancer, and/or (other) KRAS-related diseases; AR-V7 positive prostate cancer, and/or (other) AR-related diseases; basal cell cancer, Bowen's disease, hypertrophic scars, keloids, Cholangiocarcinoma, Liver cancer, obesity, bladder cancer, and/or (other) PTGS2- or TGFB1-related diseases; hepatitis B and hepatitis D virus infections, and/or (other) HBsAg-related diseases; hypertension, and/or (other) AGT-related diseases; Alport Syndrome, and/or (other) MIR21-related diseases; liver cancer, and/or (other) CEBPA-related diseases; and fibrotic scars, including hypertrophic scars and keloid, and cutaneous fibrosis, including scleroderma, and/or (other) MIR29B1-related diseases.

In certain preferred embodiments the disease or condition is selected from the group consisting of Cytomegalovirus retinitis in immunocompromised patients, Homozygous familial hypercholesterolemia, Spinal muscular atrophy, Duchenne muscular dystrophy, Veno-occlusive disease, Hereditary transthyretin-mediated amyloidosis, Acute hepatic Porphyria, Primary hyperoxaluria type 1, Primary hypercholesterolemia, Neovascular (Wet) Age-Related Macular Degeneration, Familial chylomicronemia syndrome.

In certain preferred embodiments of the invention, the method of treatment is the treatment or prophylaxis of a muscle wasting disorder, wherein the muscle wasting disorder is a muscle cell-related genetic disorder, preferably being a congenital myopathy or a muscular dystrophy; preferably wherein the congenital myopathy is selected from nemaline myopathy or congenital fiber-type disproportion myopathy, and/or wherein the muscular dystrophy is selected from a dystrophinopathy, facioscapulohumeral muscular dystrophy, myotonic dystrophy, Emery-Dreifuss muscular dystrophy, limb-girdle muscular dystrophy 1B, congenital muscular dystrophy; or dilated familial cardiomyopathy; most preferably wherein the muscle wasting disorder is a muscle cell-related genetic disorder being a dystrophinopathy, preferably being Duchenne muscular dystrophy.

In certain preferred embodiments of the invention, the method of treatment is the treatment or prophylaxis of a muscle wasting disorder, wherein the treatment or prophylaxis of the muscle wasting disorder involves antisense therapy, preferably involving exon skipping.

In certain preferred embodiments of the invention, methods of treatment as defined herein are provided, wherein the disease or condition is a disease or condition related to a defect in (the expression of) a gene or a condition is a disease or condition that is treatable by modulating the expression and/or expression level of a gene, wherein said is gene selected from the group consisting of IRS1, ICAM1, TTR, FUS, APOC3, LPA, CEP290, SOD1, HTT, TGFB2, GFAP, CCR3/CSF2RB, GHR, ITGA4, PCSK9, FOXP3, viral HBV, viral UL123, ApoB100, ARSA, ALAS, GO, VEGF, MAPT, PCED, STAT3, KLB1, DYN2, UBE2A, DGAT2, SNCA, ATXN2, LRRK2, AGT, F11, GCGR, KLBK1, AR, SCNNIA, TMPRSS6, TGFB2, DMD (dystrophin), GRB2, RHO, USH2A, SCN1A, ANGPTL3, C2orf72, SERPINC1, LDHA, CASP2, TP53, TRPV1, SERPINA1, HSD17B13, ANGPTL3, APOC3, ADRB2, SERPINA1, SERPINHI, C5, CHST15, CTGF, KRAS, PTGS2/TGFB1, HBsAG, MIR21, CEBPA, MIR29B1, ALAS1, HAO1, SMN2, APOB, CMV virus IE2, GJA1 and CFB.

In preferred embodiments of the invention, a method of treatment as defined herein is provided, wherein:
a) the disease is Cytomegalovirus retinitis in immunocompromised patients and the oligonucleotide therapeutic is selected from the group consisting of fomivirsen and therapeutically equivalent ASOs;
b) the disease is Homozygous familial hypercholesterolemia and the oligonucleotide therapeutic is selected from the group consisting of mipomersen and therapeutically equivalent ASOs;
c) the disease is Spinal muscular atrophy and the oligonucleotide therapeutic is selected from the group consisting of nusinersen and therapeutically equivalent ASOs;
d) the disease is Duchenne muscular dystrophy and the oligonucleotide therapeutic is selected from the group consisting of eteplirsen, golodirsen, viltolarsen, casimersen and therapeutically equivalent ASOs;
e) the disease is (severe) hepatic veno-occlusive disease (VOD) and the oligonucleotide therapeutic is selected from the group consisting of Defibrotide and therapeutically equivalent ASOs;
f) the disease is Hereditary transthyretin-mediated amyloidosis and the oligonucleotide therapeutic is selected from the group consisting of inotersen, patisiran, vutrisiran and therapeutically equivalent ASOs and siRNAs;
g) the disease is Acute hepatic Porphyria and the oligonucleotide therapeutic is selected from the group consisting of givosiran and therapeutically equivalent siRNAs;
h) the disease is Primary hyperoxaluria type 1 and the oligonucleotide therapeutic is selected from the group consisting of lumasiran and [therapeutically equivalent siRNAs;
i) the disease is Primary hypercholesterolemia and the oligonucleotide therapeutic is selected from the group consisting of Inclisiran and therapeutically equivalent siRNAs;
j) the disease is Neovascular (Wet) Age-Related Macular Degeneration and the oligonucleotide therapeutic is selected from the group consisting of pegaptanib and therapeutically equivalent oligonucleotides; or
k) the disease is Familial chylomicronemia syndrome and the oligonucleotide therapeutic is selected from the group consisting of volanesorsen and therapeutically equivalent ASOs.

In preferred embodiments of the invention, a method of treatment as defined herein is provided, wherein the disease relates to the vasculature and/or haemostasis, more preferably wherein:
a) the disease is Homozygous familial hypercholesterolemia and the oligonucleotide therapeutic is selected from the group consisting of mipomersen and therapeutically equivalent ASOs;
b) the disease is Acute hepatic Porphyria and the oligonucleotide therapeutic is selected from the group consisting of givosiran and therapeutically equivalent siRNAs;
c) the disease is Primary hypercholesterolemia and the oligonucleotide therapeutic is selected from the group consisting of Inclisiran and therapeutically equivalent siRNAs; or
d) the disease is Familial chylomicronemia syndrome and the oligonucleotide therapeutic is selected from the group consisting of volanesorsen and therapeutically equivalent ASOs.

In other preferred embodiments of the invention, a method of treatment as defined herein is provided, wherein the disease is Duchenne muscular dystrophy and the oligonucleotide therapeutic is selected from the group consisting of casimersen, eteplirsen, viltolarsen, golodirsen, vesleteplirsen, renardisen, NS-089 (NCP-02), PGN-EDO51, BMN 351, WVE-N531, AOC 1044, Dyne-251, RGX-202, NS-050/NCNP-03, ENTR-601-44, NS-065/NCNP-01, ataluren and ATL 102.

In other preferred embodiments of the invention, a method of treatment as defined herein is provided, wherein the oligonucleotide therapeutic and the disease are selected from the following combinations, wherein the ASO may also be a therapeutically equivalent variant of the recited ASOs:

TABLE A

| | Disease | Oligonucleotide (ASO) |
|---|---|---|
| 1 | ocular neovascularization in patients with front of the eye (cornea) | aganirsen |
| | or back of the eye (retinal) diseases, including progressive corneal | |
| | neovascularization, such as in patients with infectious keratitis and | |
| | wet age related macular degeneration (AMD), and/or (other) | |
| | IRS1-related diseases | |
| 2 | (acute disease flares in) moderate to severe Inflammatory Bowel | alicaforsen |
| | Disease (IBD), and/or (other) ICAM1-related diseases | |
| 3 | chronic heart failure and high blood pressure, particularly for | eplontersen |
| | patients with resistant hypertension due to elevated aldosterone, | |
| | and/or (other) TTR-related diseases | |
| 4 | fused in sarcoma (FUS)-protein associated myotrophic lateral | ION-363 |
| | sclerosis (ALS), and/or (other) FUS-related diseases | |
| 5 | hypertriglyceridemia, and/or (other) APOC3-related diseases | olezarsen |
| 6 | Elevated Lp(a)-associated diseases, including cardiovascular | pelacarsen |
| | disease (CVD), and/or (other) LPA-related diseases | |
| 7 | CEP290-mediated Leber congenital amaurosis 10 (LCA10), | sepofarsen |
| | and/or (other) CEP290-related diseases | |
| 8 | amyotrophic lateral sclerosis (ALS), and/or (other) SOD1-related | tofersen |
| | diseases | |
| 9 | Huntington's disease (HD), and/or (other) HTT-related diseases | tominersen |
| 10 | Cancers associated with TGFB2-overexpression, including brain | trabedersen |
| | cancer, colorectal cancer, melanoma and pancreatic cancer, | |
| | and/or (other) TGFB2-related diseases | |
| 11 | Alexander's Disease (AxD), and/or (other) GFAP-related diseases | zilganersen |
| 12 | moderate-to-severe asthma, and/or (other) CCR3- or CSF2RB- | ASM-8 |
| | related diseases | |
| 13 | excessive growth hormone (GH)-associated diseases, including | atesidorsen |
| | acromegaly, and/or (other) GHR-related diseases | |
| 14 | Duchenne muscular dystrophy (DMD), and/or (other) ITGA4- | ATL-1102 |
| | related diseases | |
| 15 | dyslipidemia, and/or (other) PCSK9-related diseases | AZD-8233 |
| 16 | cancer associated with advanced solid tumours, including clear | AZD-8701 |
| | cell renal cell cancer (ccRCC), non-small-cell lung cancer | |
| | (NSCLC), triple negative breast neoplasms (TNBN), squamous | |
| | cell cancer of head and neck (HNSCC), small cell lung cancer | |
| | (SCLC), gastroesophageal cancer, melanoma, cervical cancer, | |
| | and/or (other) FOXP3-related diseases | |
| 17 | chronic hepatitis B (CHB), and/or (other) viral HBV-related | bepirovirsen |
| | diseases | |
| 18 | Alzheimer's Disease, and/or (other) MAPT-related diseases | BIIB-080 |
| 19 | hypercholesterolaemia, and/or (other) PCSK9-related diseases | cepadacursen |
| 20 | Acromegaly, and/or (other) GHR-related diseases | cimderlirsen |
| 21 | persistent Corneal Epithelial Defects (PCED), and/or (other) | CODA-001 |
| | GJA1-related diseases | |
| 22 | Cancers involving STAT, including lymphoma, lung cancer and | danvatirsen |
| | head and neck squamous cell carcinoma (HNSCC), and/or (other) | |
| | STAT3-related diseases | |
| 23 | hereditary angioedema (HAE), and/or (other) KLKB1-related | donidalorsen |
| | diseases | |
| 24 | congenital structural myopathies, and/or (other) DYN2-related | DYN-101 |
| | diseases | |
| 25 | angelman syndrome (AS), and/or (other) UBE2A-related diseases | GTX-102 |
| 26 | nonalcoholic steatohepatitis (NASH), and/or (other) DGAT2- | ION-224 |
| | related diseases | |
| 27 | gastrointestinal autoimmune diseases | ION-253 |
| 28 | Parkinson's disease (PD), multiple system atrophy (MSA) and | ION-464 |
| | related synucleinopathies, and/or (other) SNCA-related diseases | |
| 29 | amyotrophic lateral sclerosis (ALS), and/or (other) ATXN2-related | ION-541 |
| | diseases | |
| 30 | Parkinson's disease (PD), and/or (other) LRRK2-related diseases | ION-859 |
| 31 | hypertension and/or chronic heart failure, and/or (other) AGT- | IONIS-AGTLRx |
| | related diseases | |
| 32 | complement-mediated diseases, including IgA nephropathy and | IONIS-FB-LRx |
| | Age-related macular degeneration (AMD) | |
| 33 | clotting disorders, including thrombosis, and end-stage renal | IONIS-FXILRx |
| | disease (ESRD), and/or (other) F11-related diseases | |
| 34 | type 2 Diabetes, and/or (other) GCGR-related diseases | IONIS-GCGRRx |
| 35 | hepatitis B virus (HBV) infections, and/or (other) viral HBV-related | IONIS-HBVLRx |
| | diseases | |
| 36 | hereditary angioedema (HAE), and/or (other) KLKB1-related | IONIS-PKKRx |
| | diseases | |
| 37 | prostate cancer, and/or (other) AR-related diseases | IONISAR-2.5Rx |
| 38 | cystic fibrosis, and/or (other) SCNN1A-related diseases | IONISENAC-2.5Rx |
| 39 | beta-thalassemia, and/or (other) TMPRSS6-related diseases | IONISTMPRSS-6LRx |
| 40 | primary open-angle glaucoma (POAG), and/or (other) TGFB2- | ISTH-0036 |
| | related diseases | |
| 41 | Duchenne Muscular Dystrophy, and/or (other) DMD-related | NS-089 |
| | diseases | |
| 42 | RAS-activated cancers, including chronic myeloid leukaemia | prexigebersen |
| | (CML), acute myeloid leukemia (AML), acute lymphocytic | |
| | leukemia (ALL) and myelodysplastic syndromes (MDS), and/or | |
| | (other) GRB2-related diseases | |
| 43 | retinitis pigmentosa (RP), and/or (other) RHO-related diseases | QR-1123 |
| 44 | retinitis pigmentosa (RP), and/or (other) USH2A-related diseases | QR-421a or |
| | | ultevursen |
| 45 | Duchenne muscular dystrophy (DMD), and/or (other) DMD-related | renadirsen |
| | diseases | |
| 46 | Duchenne muscular dystrophy (DMD), and/or (other) DMD-related | SRP-5051 |
| | diseases | |
| 47 | dravet syndrome, and/or (other) SCN1A-related diseases | STK-001 |
| 48 | diabetes, hepatic steatosis, and hypertriglyceridaemia, and/or | vupanorsen |
| | (other) ANGPTL3-related diseases | |
| 49 | cardiovascular diseases, and/or (other) HTT-related diseases | WVE-003 |
| 50 | amyotrophic lateral sclerosis (ALS) and frontotemporal disorders | WVE-004 |
| | (FTD), and/or (other) C9orf72-related diseases | |
| 51 | Duchenne muscular dystrophy, and/or (other) DMD-related | WVE-N531 |
| | diseases | |

In other preferred embodiments of the invention, a method of treatment as defined herein is provided, wherein the oligonucleotide therapeutic and the disease relate to the vasculature and/or haemostasis, and are selected from the following combinations, wherein the ASO may also be a therapeutically equivalent variant of the recited ASOs:

TABLE AA

| | Disease | Oligonucleotide (ASO) |
|---|---|---|
| 1 | chronic heart failure and high blood pressure, particularly for | eplontersen |
| | patients with resistant hypertension due to elevated aldosterone, | |
| | and/or (other) TTR-related diseases | |
| 2 | hypertriglyceridemia, and/or (other) APOC3-related diseases | olezarsen |
| 3 | Elevated Lp(a)-associated diseases, including cardiovascular | pelacarsen |
| | disease (CVD), and/or (other) LPA-related diseases | |
| 4 | dyslipidemia, and/or (other) PCSK9-related diseases | AZD-8233 |
| 5 | hypercholesterolaemia, and/or (other) PCSK9-related diseases | cepadacursen |
| 6 | hypertension and/or chronic heart failure, and/or (other) AGT- | IONIS-AGTLRx |
| | related diseases | |
| 7 | clotting disorders, including thrombosis, and end-stage renal | IONIS-FXILRx |
| | disease (ESRD), and/or (other) F11-related diseases | |
| 8 | type 2 Diabetes, and/or (other) GCGR-related diseases | IONIS-GCGRRx |
| 9 | beta-thalassemia, and/or (other) TMPRSS6-related diseases | IONISTMPRSS-6LRx |
| 10 | diabetes, hepatic steatosis, and hypertriglyceridaemia, and/or | vupanorsen |
| | (other) ANGPTL3-related diseases | |
| 11 | cardiovascular diseases, and/or (other) HTT-related diseases | WVE-003 |

In another preferred embodiment of the invention, a method of treatment as defined herein is provided, wherein the oligonucleotide therapeutic is IONIS-FXILRx or a therapeutically equivalent variant thereof, and the disease relates to haemostasis and is selected from clotting disorders, including thrombosis, and end-stage renal disease (ESRD), and/or (other) FLD1-related diseases.

In other preferred embodiments of the invention, a method of treatment as defined herein is provided, wherein the oligonucleotide therapeutic and the disease are selected from the following combinations, wherein the siRNA may also be a therapeutically equivalent variant of the recited siRNA:

TABLE B

| | Disease | Oligonucleotide (siRNA) |
|---|---|---|
| 1 | hemophilia A or B, and/or (other) SERPINC1-related diseases | fitusiran |
| 2 | primary hyperoxaluria (PH), and/or (other) LDHA-related diseases | nedosiran |
| 3 | optic neuropathies including glaucoma, and/or (other) CASP2- | QPI-1007 |
| | related diseases | |
| 4 | optic neuropathies including glaucoma, and/or (other) TP53- | teprasiran |
| | related diseases | |
| 5 | dry eye disease, and/or (other) TRPV1-related diseases | tivanisiran |
| 6 | hepatitis B virus (HBV) infections, and/or (other) HBsAg-related | AB-729 |
| | diseases | |
| 7 | gastrointestinal and metabolic disorders, and/or (other) | ALNAAT-02 |
| | SERPINA1-related diseases | |
| 8 | gastrointestinal disorders including non-alcoholic steatohepatitis, | ARO-HSD |
| | and/or (other) HSD17B13-related diseases | |
| 9 | homozygous familial hypercholesterolemia, and/or (other) | AROANG-3 |
| | ANGPTL3-related diseases | |
| 10 | familial chylomicronemia syndrome (FCS), and/or (other) APOC3- | AROAPOC-3 |
| | related diseases | |
| 11 | glaucoma and ocular hypertension, and/or (other) ADRB2-related | bamosiran |
| | diseases | |
| 12 | alpha-1 antitrypsin (AAT) deficiency-associated liver disease | belcesiran |
| | (AATLD), and/or (other) SERPINA1-related diseases | |
| 13 | advanced hepatic fibrosis, and/or (other) SERPINH1-related | BMS-986263 |
| | diseases | |
| 14 | immunoglobulin A nephropathy, and/or (other) C5-related | cemdisiran |
| | diseases | |
| 15 | alpha-1 antitrypsin (AAT) deficiency-associated liver disease | fazirsiran |
| | (AATLD), and/or (other) SERPINA1-related diseases | |
| 16 | hepatitis B virus (HBV) infections, and/or (other) viral HBV-related | JNJ-3989 |
| | diseases | |
| 17 | ulcerative colitis (=chronic inflammatory bowel disease (IBD), | MT-5745 |
| | and/or (other) CHST15-related diseases | |
| 18 | atherosclerotic cardiovascular diseases, and/or (other) LPA- | olpasiran |
| | related diseases | |
| 19 | hypertrophic and keloid scars, and/or (other) CTGF-related | OLX-101 |
| | diseases | |
| 20 | chronic hepatitis B virus (HBV) infection, and/or (other) HBsAg- | RG-6346 |
| | related diseases | |
| 21 | pancreatic cancer, and/or (other) KRAS-related diseases | siG-12D-LODER |
| 22 | AR-V7 positive prostate cancer, and/or (other) AR-related | SR-063 |
| | diseases | |
| 23 | basal cell cancer, Bowen's disease, hypertrophic scars, keloids, | STP-705 |
| | cholangiocarcinoma, liver cancer, obesity, bladder cancer, and/or | |
| | (other) PTGS2- or TGFB1-related diseases | |
| 24 | hepatitis B and hepatitis D virus infections, and/or (other) HBsAg- | VIR-2218 |
| | related diseases | |
| 25 | hypertension, and/or (other) AGT-related diseases | zilebesiran |

In other preferred embodiments of the invention, a method of treatment as defined herein is provided, wherein the oligonucleotide therapeutic and the disease relate to the vasculature and/or haemostasis, and are selected from the following combinations, wherein the siRNA may also be a therapeutically equivalent variant of the recited siRNA:

TABLE BB

| | | Oligo- |
|---|---|---|
| | | nucleotide |
| | Disease | (siRNA) |
| 1 | hemophilia A or B, and/or (other) SERPINC1- | fitusiran |
| | related diseases | |
| 2 | homozygous familial hypercholesterolemia, and/or | AROANG-3 |
| | (other) ANGPTL3-related diseases | |
| 3 | familial chylomicronemia syndrome (FCS), and/or | AROAPOC-3 |
| | (other) APOC3-related diseases | |
| 4 | atherosclerotic cardiovascular diseases, and/or | olpasiran |
| | (other) LPA-related diseases | |
| 5 | hypertension, and/or (other) AGT-related diseases | zilebesiran |

In another preferred embodiment of the invention, a method of treatment as defined herein is provided, wherein the oligonucleotide therapeutic is fitusiran or a therapeutically equivalent variant thereof and the disease relates to haemostasis, and is selected from hemophilia A, hemophilia B, and (other) SERPINC1-related diseases.

In other preferred embodiments of the invention, a method of treatment as defined herein is provided, wherein the oligonucleotide therapeutic and the disease are selected from the following combinations:

TABLE C

| | | Oligo- |
|---|---|---|
| | Disease | nucleotide |
| 1 | Alport Syndrome, and/or (other) MIR21-related | lademirsen |
| | diseases | |
| 2 | liver cancer, and/or (other) CEBPA-related | MTL-CEBPA |
| | diseases | |
| 3 | fibrotic scars, including hypertrophic scars | remlarsen |
| | and keloid, and cutaneous fibrosis, including | |
| | scleroderma, and/or (other) MIR29B1-related | |
| | diseases | |

In an embodiment, the method of treatment is the treatment or prophylaxis of a muscle wasting disorder, wherein the saponin component comprises a ligand for an endocytic receptor on a muscle cell and/or the nucleic acid component comprises a ligand for an endocytic receptor on a muscle cell, wherein the endocytic receptor is preferably selected from: transferrin receptor (CD71), insulin-like growth factor 1 (IGF-1) receptor (IGF1R), tetraspanin CD63; muscle-specific kinase (MuSK), glucose transporter GLUT4, cation independent mannose 6 phosphate receptor (CI-MPR), and LDL receptor.

An embodiment is the combination of the saponin component and the nucleic acid component for use in the treatment or prophylaxis of a muscle wasting disorder, wherein the saponin component comprises a ligand for an endocytic receptor on a muscle cell and/or the nucleic acid component comprises a ligand for an endocytic receptor on a muscle cell, wherein the ligand is selected from any one of:
insulin-like growth factor 1 (IGF-1) or fragments thereof;
insulin-like growth factor 2 (IGF-II) or fragments thereof;
mannose 6 phosphate;
transferrin (Tf);
zymozan A; and
an antibody or a binding fragment thereof specific for binding to the endocytic receptor, wherein the endocytic receptor is preferably selected from: transferrin receptor (CD71), insulin-like growth factor 1 (IGF-1) receptor (IGF1R), tetraspanin CD63, muscle-specific kinase (MuSK), glucose transporter GLUT4, cation independent mannose 6 phosphate receptor (CI-MPR), and LDL receptor; preferably wherein the ligand is an antibody or a binding fragment thereof that is specific for binding to a transferrin receptor,
more preferably wherein the ligand is a monoclonal antibody or a Fab′ fragment or at least one single domain antibody specific for binding to a transferrin receptor, even more preferably wherein the ligand is a monoclonal antibody specific for binding to a transferrin receptor.

In an embodiment, the method of treatment is the treatment or prophylaxis of a muscle wasting disorder, wherein the nucleic acid comprised by the nucleic acid component is defined as a nucleic acid that is no longer than 150 nt, preferably wherein the oligonucleotide has a size of 5-150 nt, preferably being 8-100 nt, most preferably being 10-50 nt.

In an embodiment, the method of treatment is the treatment or prophylaxis of a muscle wasting disorder, wherein the nucleic acid comprised by the nucleic acid component is an antisense oligonucleotide, preferably being a mutation specific antisense oligonucleotide, most preferably being an oligonucleotide designed to induce exon skipping.

In an embodiment, the method of treatment is the treatment or prophylaxis of a muscle wasting disorder, wherein the nucleic acid comprised by the nucleic acid component comprises or consists of any one of the following: morpholino phosphorodiamidate oligomer (PMO), 2′-O-methyl (2′-OMe) phosphorothioate RNA, 2′-O-methoxyethyl (2′-O-MOE) RNA {2′-O-methoxyethyl-RNA (MOE)}, locked/bridged nucleic acid (BNA), 2′-0,4′-aminoethylene bridged nucleic acid (BNANC), peptide nucleic acid (PNA), 2′-deoxy-2′-fluoroarabino nucleic acid (FANA), 3′-fluoro hexitol nucleic acid (FHNA), glycol nucleic acid (GNA), threose nucleic acid (TNA), silencing RNA (siRNA), short hairpin RNA (shRNA), microRNA (miRNA), antagomir (miRNA antagonists), aptamer RNA or aptamer DNA, single-stranded RNA or single-stranded DNA, double-stranded RNA (dsRNA) or double-stranded DNA; preferably wherein the nucleic acid comprises or consists of a morpholino phosphorodiamidate oligomer (PMO) or a 2′-O-methyl (2′-OMe) phosphorothioate RNA or a 2MOE (2′-O-(2-Methoxyethyl)-oligoribonucleotides (2′-O-MOE bases)).

In an embodiment, the method of treatment is the treatment or prophylaxis of a muscle wasting disorder, wherein the nucleic acid comprised by the nucleic acid component is designed to induce exon skipping of human dystrophin gene transcript, preferably wherein the exon skipping involves exon 51 skipping or exon 53 skipping or exon 45 skipping; more preferably wherein the nucleic acid is a 2′O-methyl-phosporothioate antisense oligonucleotide or a phosphorodiamidate morpholino oligomer antisense oligonucleotide that is designed to induce the exon 51 skipping or the exon 53 skipping or the exon 45 skipping,
even more preferably wherein the nucleic acid is selected from eteplirsen, drisapersen, golodirsen, viltolarsen, and casimersen.

In an embodiment, the method of treatment is the treatment or prophylaxis of a muscle wasting disorder, wherein the composition comprises two or more different oligonucleotides comprised by the nucleic acid component, preferably wherein at least one of the two or more different oligonucleotides is an antisense oligonucleotide.

In a preferred embodiment, the muscle wasting disorder is Duchenne muscular dystrophy. In a particularly preferred embodiment, the muscle wasting disorder is Duchenne muscular dystrophy and the effector component is an ASO or a PMO, more preferably wherein the effector component is selected from eteplirsen, drisapersen, golodirsen, viltolarsen, and casimersen.

In preferred embodiments of the invention, a method of treatment as defined herein is provided, wherein the administration of the saponin component results in an extension of the effect of the effector component or effector moiety, and/or in an extension of the dosing interval of the effector component or effector moiety and/or in a reduction of the dosing frequency of the effector component or effector moiety and/or a (delayed) boost in the effect of the effector component or effector moiety.

In preferred embodiments of the invention, a method of treatment as defined herein is provided, wherein said method, especially in case the effector component is a nucleic acid or oligonucleotide therapeutic, results in an extension of the effect of the effector component and/or an extension of the dosing interval of the effector component by a factor 1.25 or more, preferably by a factor 1.5 or more, by a factor 1.75 or more, by a factor 2 or more, by a factor 2.25 or more, by a factor 2.5 or more, by a factor 2.75, or by a factor 3 or more. There is no particular upper limit, as will be understood by those skilled in the art, but the inventors currently assume that the present methods, in most instances, typically will extend the effect of the effector component and/or allow for an extension of the dosing interval of the effector component by not more than a factor 5. In preferred embodiments of the invention, a method of treatment as defined herein is provided, wherein said method, especially in case the effector component is a nucleic acid or oligonucleotide therapeutic, results in reduction of the dosing frequency of the effector component by a factor of 0.90 or lower, preferably 0.85 or lower, 0.80 or lower, 0.75 or lower, 0.70 or lower, 0.65 or lower, 0.60 or lower, 0.55 or lower, 0.50 or lower, 0.45 or lower, 0.40 or lower, or 0.35 or lower. There is no particular lower limit, but the inventors currently assume that the present methods, in most instances, will typically allow for a reduction in the dosing frequency of the effector component by not less than a factor 0.20.

As used herein the term “(delayed) boost in the effect of the effector component or effector moiety” refers to the phenomenon that the administration of the saponin component, typically some time after the effector component was administered, results in the release of the effector component or moiety from the endosomes, thereby increasing/enhancing the pharmacological effect of the effector molecule or moiety. Advantageously, the administration of the saponin component takes place (some time) after the peak in the pharmacological effect, following the administration of the effector component, has been reached, and the administration of the saponin component results in a second/further peak in the pharmacological effect. As will be understood by those skilled in the art, based on the present teachings, the saponin component can be used to induce this effect (some time) after administration of the effector component, more than once.

In an embodiment of the invention, a method of treatment as defined herein is provided, wherein the effector component is or comprises an oligonucleotide selected from: deoxyribonucleic acid (DNA) oligomer, ribonucleic acid (RNA) oligomer, antisense oligonucleotide (ASO, AON), short interfering RNA (siRNA), anti-microRNA (anti-miRNA), DNA aptamer, RNA aptamer, mRNA, mini-circle DNA, peptide nucleic acid (PNA), phosphoramidate morpholino oligomer (PMO), phosphorothioate-modified antisense oligonucleotide (PS-ASO), 2′-O-methyl (2′-OMe) phosphorothioate RNA, 2′-O-methoxyethyl (2′-O-MOE) RNA {2′-O-methoxyethyl-RNA (MOE)}, locked nucleic acid (LNA), bridged nucleic acid (BNA), 2′-deoxy-2′-fluoroarabino nucleic acid (FANA), 2′-O-methoxyethyl-RNA (MOE), 3′-fluoro hexitol nucleic acid (FHNA), glycol nucleic acid (GNA), xeno nucleic acid oligonucleotide and threose nucleic acid (TNA). Preferred is an siRNA for silencing apolipoprotein B. Also preferred is an siRNA for silencing transthyretin. Also preferred is a PMO designed to induce exon skipping.

In an embodiment of the invention, a method of treatment as defined herein is provided, wherein the effector component is or comprises an oligonucleotide selected from any one or more of a(n): short interfering RNA (siRNA), short hairpin RNA (shRNA), anti-hairpin-shaped microRNA (miRNA), single-stranded RNA, aptamer RNA, double-stranded RNA (dsRNA), anti-microRNA (anti-miRNA, anti-miR), antisense oligonucleotide (ASO), mRNA, DNA, antisense DNA, locked nucleic acid (LNA), bridged nucleic acid (BNA), 2′-0,4′-aminoethylene bridged nucleic Acid (BNANC), BNA-based siRNA, and BNA-based antisense oligonucleotide (BNA-AON).

In an embodiment of the invention, a method of treatment as defined herein is provided, wherein the effector component is or comprises an oligonucleotide selected from any one of: an anti-miRNA, a BNA-AON or an siRNA, such as BNA-based siRNA, preferably selected from chemically modified siRNA, metabolically stable siRNA and chemically modified, metabolically stable siRNA.

In a preferred embodiment of the invention, a method of treatment as defined herein is provided, wherein the effector component is an advanced ESC siRNA or wherein the effector component is an advanced ESC GalNAc-conjugated siRNA, preferably comprising one or three GalNAc moieties, more preferably three GalNAc moieties.

An embodiment of the invention is the provision of a method of treatment as defined herein, wherein the effector component is or comprises an oligonucleotide that is no longer than 150 nt, preferably wherein the oligonucleotide has a size of 5-150 nt, preferably being 8-100 nt, most preferably being 10-50 nt.

In an embodiment of the invention, a method of treatment as defined herein is provided, wherein the effector component is or comprises an antisense oligonucleotide, preferably being a mutation specific antisense oligonucleotide, most preferably being an oligonucleotide designed to induce exon skipping. Preferably, the nucleic acid comprised by the nucleic acid component comprises or consists of a morpholino phosphorodiamidate oligomer (PMO) or a 2′-O-methyl (2′-OMe) phosphorothioate RNA.

In an embodiment of the invention, a method of treatment as defined herein is provided, wherein the effector component is or comprises a toxin selected from: a viral toxin, a bacterial toxin, a plant toxin including ribosome-inactivating proteins and the A chain of type 2 ribosome-inactivating proteins, an animal toxin, a human toxin and a fungal toxin, more preferably the toxin is a plant toxin including ribosome-inactivating proteins and the A chain of type 2 ribosome-inactivating proteins.

In an embodiment, a method of treatment as defined herein is provided, wherein the effector component is or comprises a toxin selected from: apoptin, Shiga toxin, Shiga-like toxin, Pseudomonas aeruginosa exotoxin (PE), full-length or truncated diphtheria toxin (DT), cholera toxin, alpha-sarcin, dianthin, saporin, bouganin, de-immunized derivative debouganin of bouganin, Shiga-like toxin A, pokeweed antiviral protein, ricin, ricin A chain, modeccin, modeccin A chain, abrin, abrin A chain, volkensin, volkensin A chain, viscumin, viscumin A chain, frog RNase, granzyme B, human angiogenin; preferably the toxin is dianthin and/or saporin.

In an embodiment, a method of treatment as defined herein is provided, wherein the effector component is or comprises a toxin selected from: a toxin targeting ribosomes, a toxin targeting elongation factors, a toxin targeting tubulin, a toxin targeting DNA and a toxin targeting RNA, more preferably the toxin is selected from the list consisting of: emtansine, pasudotox, maytansinoid derivative DM1, maytansinoid derivative DM4, monomethyl auristatin E (MMAE, vedotin), monomethyl auristatin F (MMAF, mafodotin), a calicheamicin, N Acetyl-γ calicheamicin, a pyrrolobenzodiazepine (PBD) dimer, a benzodiazepine, a CC-1065 analogue, a duocarmycin, doxorubicin, paclitaxel, docetaxel, cisplatin, cyclophosphamide, etoposide, docetaxel, 5-fluorouracyl (5-FU), mitoxantrone, a tubulysin, an indolinobenzodiazepine, AZ13599185, a cryptophycin, rhizoxin, methotrexate, an anthracycline, a camptothecin analogue, SN 38, DX 8951f, exatecan mesylate, truncated form of Pseudomonas aeruginosa exotoxin (PE38), a duocarmycin derivative, an amanitin, a amanitin, a spliceostatin, a thailanstatin, ozogamicin, tesirine, Amberstatin269 and soravtansine.

In other specific embodiments of the invention, a method of treatment as defined herein is provided, wherein the effector component, the saponin component and the disease are selected from the following combinations:

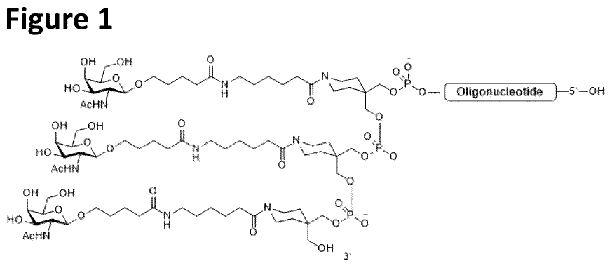

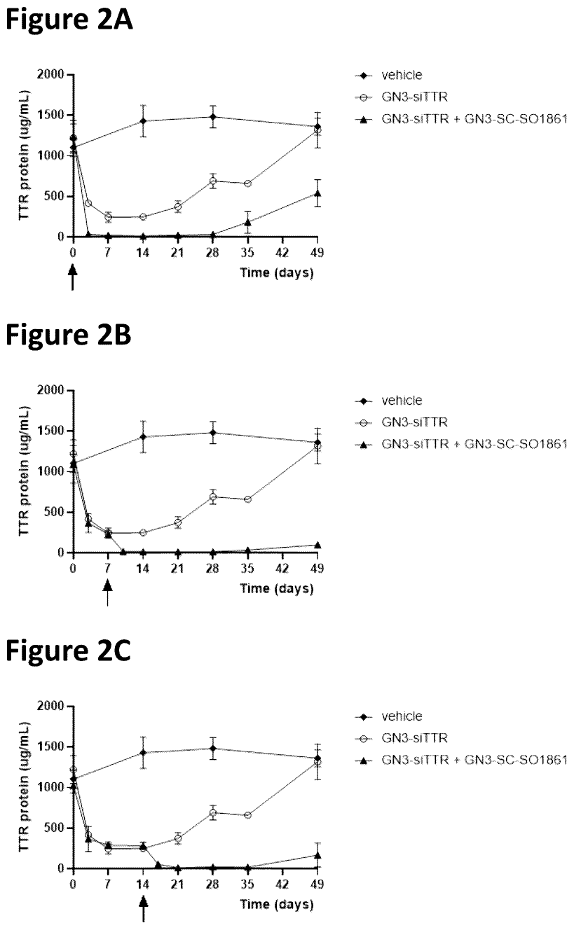

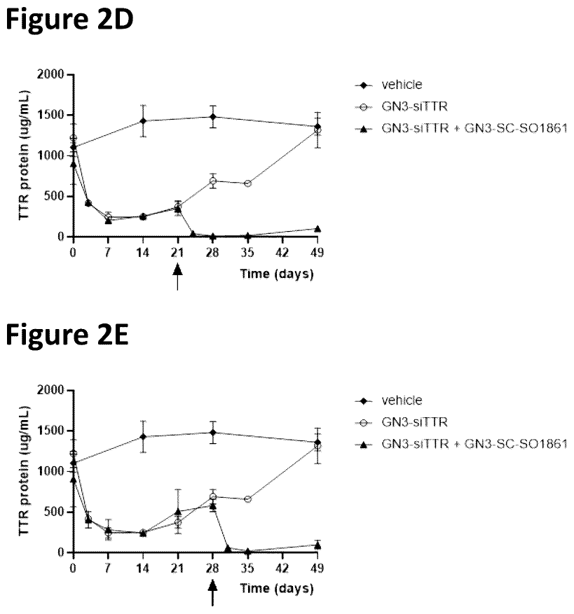

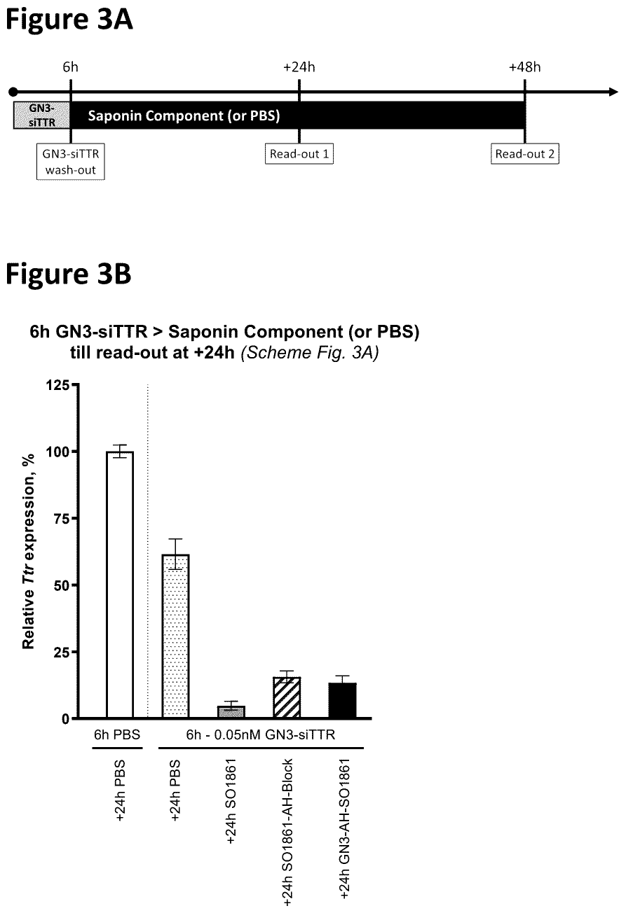

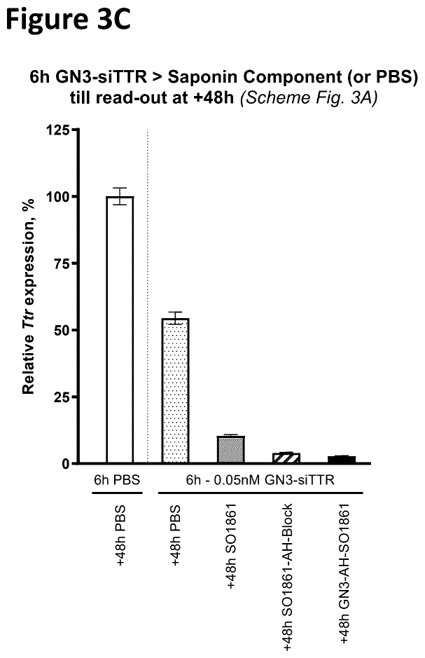

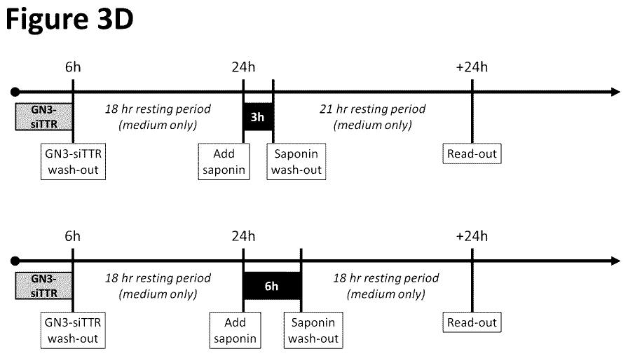

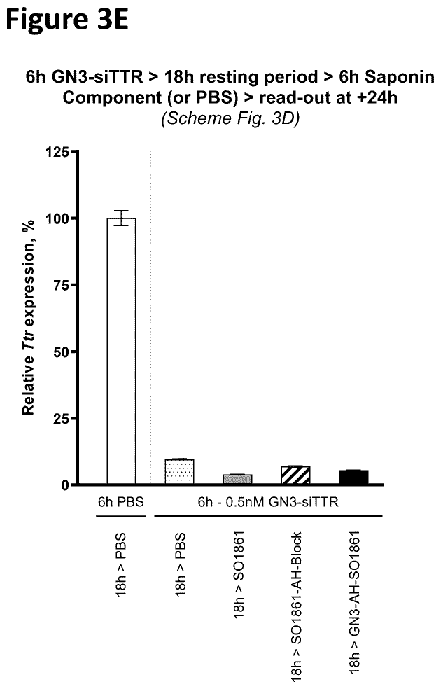

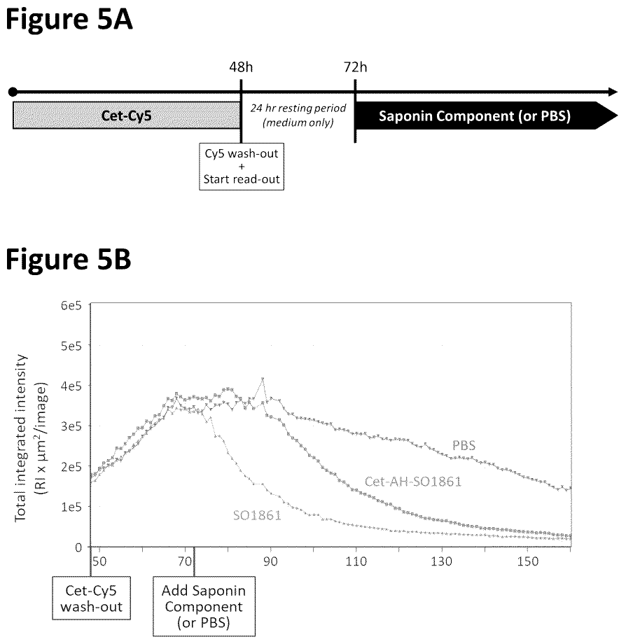

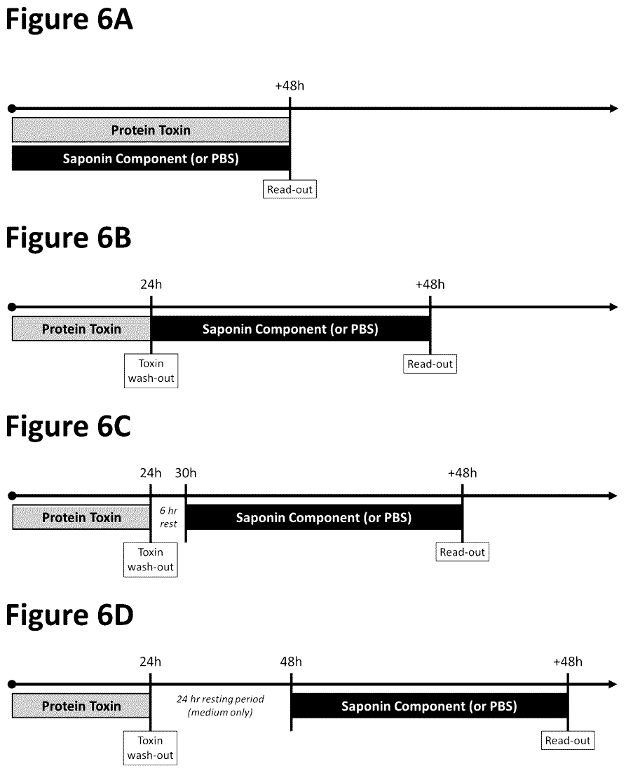

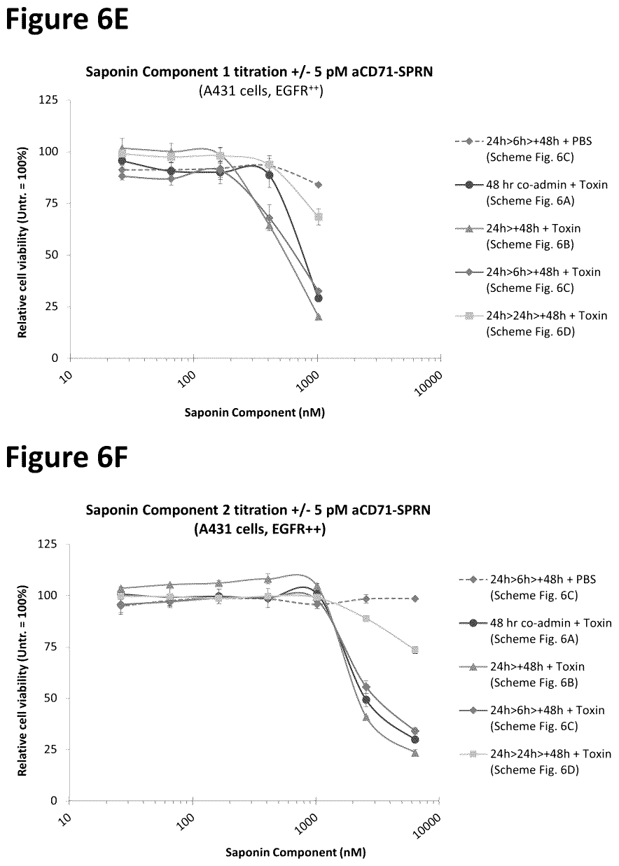

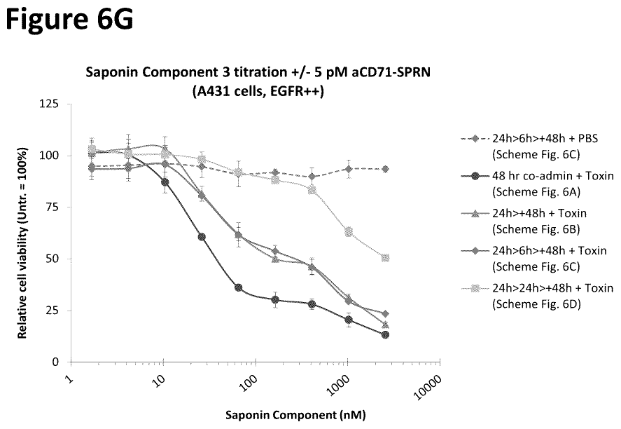

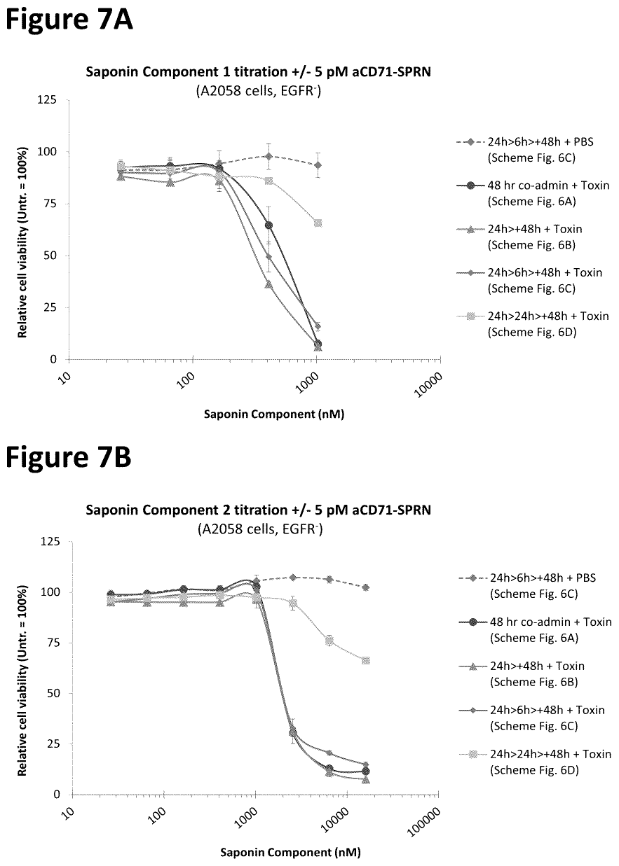

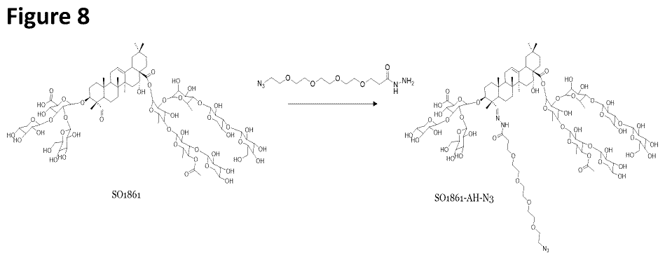

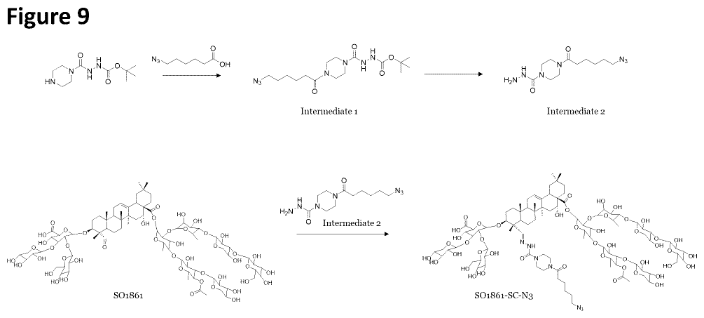

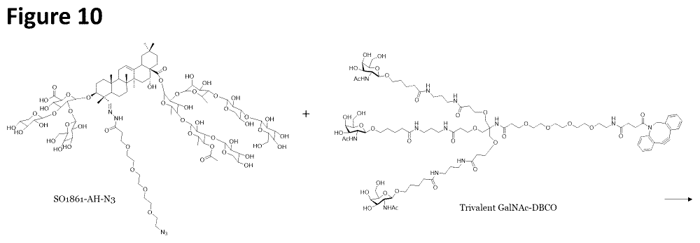

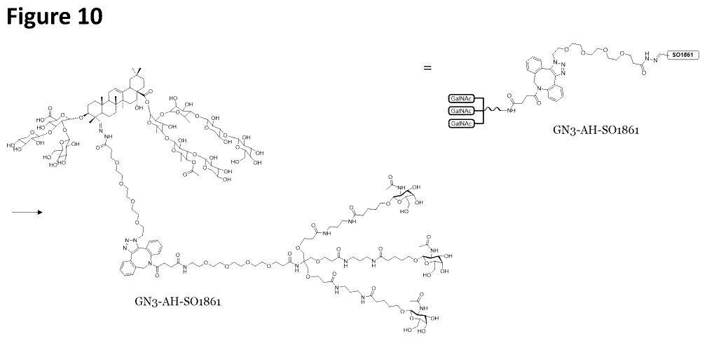

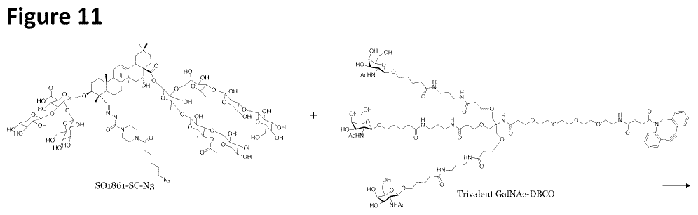

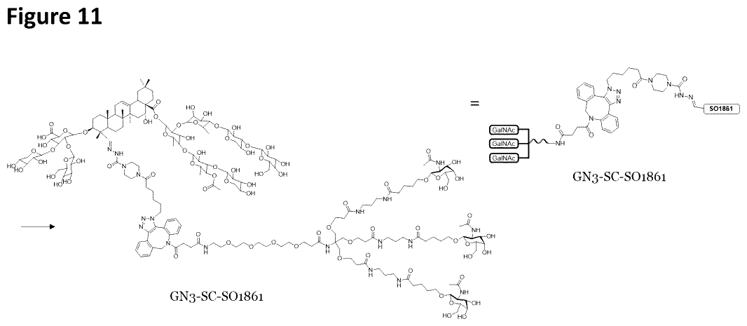

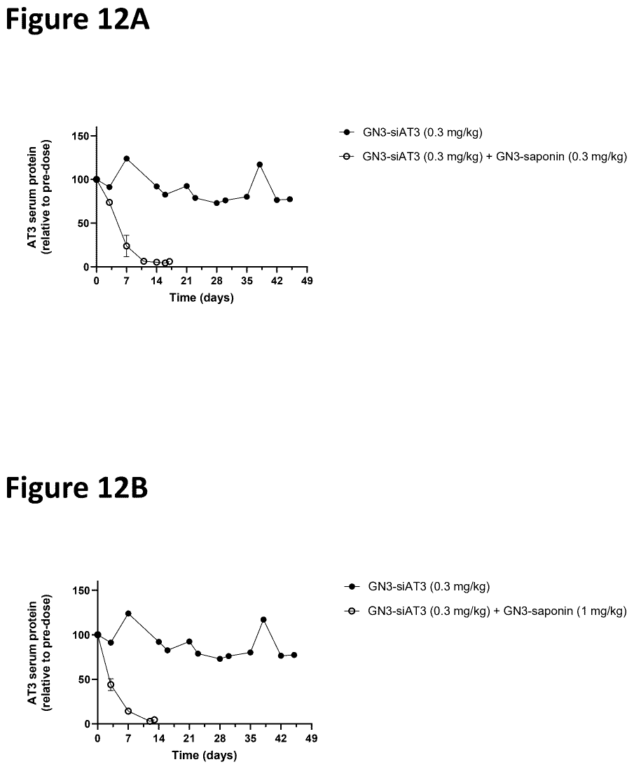

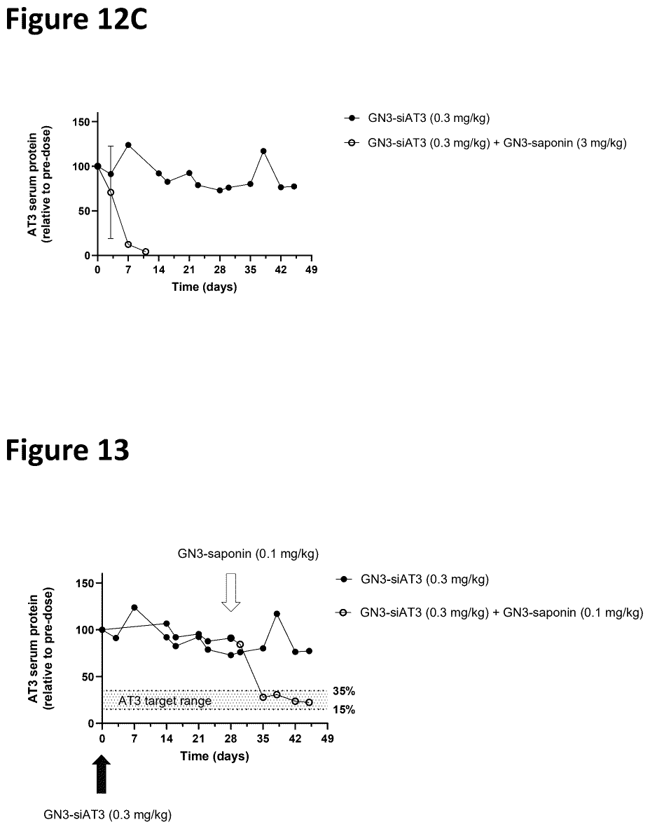

TABLE D
Working examples of combinations of saponin component and effector component
for use in the treatment or prophylaxis of indicated diseases

| | | | | Reference | Example |
|---|---|---|---|---|---|
| Effector | Saponin | Target cell and/or | Disease or | (international | details in |
| component^{‡} | component^{†} | target organ | treatment | publication) | reference |
| Trastuzumab- | Cetuximab- | Epithelial cells of | Epidermoid | WO2020126627 | Table A, |
| saporin | SO1861 | epidermis | carcinoma | | pag. 95 |
| Trastuzumab- | SO1861 | Solid, invasive | Breast cancer | WO2020126609 | FIG. 1-2 |
| saporin | | ductal carcinoma | | | |
| Cetuximab- | Cetuximab- | Epithelial cells of | Metastatic | WO2020126609 | FIG. 3-1A, |
| saporin | SO1861 | the breast | adenocarcinoma of | | FIG. 9-5A |
| | | | the breast | | |
| Trastuzumab- | Trastuzumab- | Epithelial cells | Breast cancer | WO2020126609 | FIG. 3-1B, |
| saporin | SO1861 | | | | FIG. 9-5C |
| Anti-CD71 | Cetuximab- | Epithelial cells of | Metastatic | WO2020126609 | FIG. 4-1A, |
| mAb-saporin | SO1861 | the breast | adenocarcinoma of | | FIG. 11-6A |
| | | | the breast | | |
| EGF-dianthin | SO1832 | Cervix | Cervical cancer | WO2020126609 | FIG. 2-2A, |
| | | | | | B, C |
| EGF-dianthin | SO1861 | Cervix | Cervical cancer | WO2020126609 | FIG. 2-2A, |
| | | | | | B, C |
| EGF-dianthin | SO1862 | Cervix | Cervical cancer | WO2020126609 | FIG. 2-2A, |
| | | | | | B, C |
| EGF-dianthin | SO1904 | Cervix | Cervical cancer | WO2020126609 | FIG. 2-2A, |
| | | | | | B, C |
| EGF-dianthin | GE1741 | Cervix | Cervical cancer | WO2020126609 | FIG. 2-2A, |
| | | | | | B, C |
| EGF-dianthin | Molecule of | Cervix | Cervical cancer | WO2020126609 | FIG. 3-2B |
| | formula (I)^{¶} | | | | |
| HSP27 BNA | Cetuximab- | Epithelial cells of | Epidermoid | WO2020126609 | FIG. 5-2A, |
| | SO1861 | epidermis | carcinoma | | C, FIG. 6-2 |
| Cetuximab- | Cetuximab- | Epithelial cells of | Epidermoid | WO2020126609 | FIG. 1-5, |
| HSP27 BNA | SO1861 | epidermis | carcinoma | | FIG. 8-5B, D |
| Anti-CD71 | Cetuximab- | Epithelial cells of | Epidermoid | WO2020126609 | FIG. 1-6, |
| mAb-saporin | SO1861 | epidermis | carcinoma | | FIG. 2-6, |
| | | | | | FIG. 12-6 |
| Trastuzumab- | Cetuximab- | Epidermoid cell, | Cervical cancer | WO2020126609 | FIG. 3-6A, B |
| saporin | SO1861 | HPV-16 positive | | | |
| EGF-dianthin | Trastuzumab- | Epithelial cells | Breast cancer | WO2020126609 | FIG. 5-6A, B |
| | SO1861 | | | | |
| Trastuzumab- | Cetuximab- | Epithelial cells of | Epidermoid | WO2020126609 | FIG. 10-6A, |
| HSP27 BNA | SO1861 | epidermis | carcinoma | | C |
| HSP27 BNA | Molecule of | Epithelial cells of | Epidermoid | WO2020126609 | FIG. 1-7A, C |
| | formula (I) | epidermis | carcinoma | | |
| Cetuximab- | Molecule of | Epithelial cells of | Epidermoid | WO2020126609 | FIG. 1-7A, C |
| HSP27 BNA | formula (I) | epidermis | carcinoma | | |
| Bivalent VHH | Molecule of | Epithelial cells of | Epidermoid | | |
| (anti-EGFR)- | formula (I) | epidermis | carcinoma | | |
| HSP27 BNA | | | | | |
| Anti-CD71 | Bivalent VHH | Epithelial cells of | Epidermoid | | |
| mAb-saporin | (anti-EGFR)- | epidermis | carcinoma | | |
| | SO1861 | | | | |
| Cetuximab- | Molecule of | Epithelial cells of | Epidermoid | | |
| saporin | formula (I) | epidermis | carcinoma | | |
| Anti-CD71 | Anti-Her2 VHH- | Epithelial cells | Breast cancer | | |
| VHH-dianthin | SO1861 | | | | |
| Bivalent VHH | Molecule of | Epithelial cells of | Epidermoid | | |
| (anti-EGFR)- | formula (I) | epidermis | carcinoma | | |
| dianthin | | | | | |
| EGF-dianthin | Molecule of | Cervix | Cervical cancer | WO2021260054 | FIG. 18A |
| | formula (III)^{¶} | | | | |
| EGF-dianthin | Molecule of | Cervix | Cervical cancer | WO2021260054 | FIG. 18B |
| | formula 3^{¶} | | | | |
| EGF-dianthin | Molecule of | Cervix | Cervical cancer | WO2021260054 | FIG. 19B |
| | formula V^{¶} | | | | |
| Anti-CD71 | Tri-GalNAc- | Hepatocytes | | WO2021261998 | FIG. 10A, B |
| amAb-saporin | SO1861 | | | | |
| Tri-GalNAc- | Molecule of | Hepatocytes | | WO2021261998 | FIG. 11A, B |
| HSP27 BNA | formula (I) | | | | |
| Tri-GalNAc- | Molecule of | Hepatocytes | Lowering LDL- | WO2021261998 | FIG. 12A, |
| ApoB BNA | formula (I) | | cholesterol levels in | | FIG. 14A, B, |
| | | | plasma | | FIG. 18A, B, |
| | | | | | FIG. 19A |
| Tri-GalNAc- | Tri-GalNAc- | Hepatocytes | Lowering LDL- | WO2021261998 | FIG. 16A, B |
| ApoB BNA | SO1861 | | cholesterol levels in | | |
| | | | plasma | | |
| Tri-GalNAc- | Mono-GalNAc- | Hepatocytes | Lowering LDL- | WO2021261998 | FIG. 17A |
| ApoB BNA | SO1861 | | cholesterol levels in | | |
| | | | plasma | | |
| Tri-GalNAc- | Tri-GalNAc- | Hepatocytes | Lowering LDL- | WO2021261992 | FIG. 19, |
| ApoB BNA | SO1861 | | cholesterol levels in | | FIG. 20, |
| | | | plasma | | FIG. 21 |
| EGF-dianthin | SO1861 | Cervix | Cervical cancer | WO2022164316 | FIG. 10A, B |
| EGF-dianthin | Molecule of | Cervix | Cervical cancer | WO2022164316 | FIG. 10A, B |
| | formula (I) | | | | |
| EGF-dianthin | SO1861-linker | Cervix | Cervical cancer | WO2022164316 | FIG. 10A, B |
| | via a | | | | |
| | semicarbazone | | | | |
| | bond linked to | | | | |
| | C-4 of the | | | | |
| | aglycone | | | | |
| Trastuzumab- | Molecule of | Epithelial cells of | Epidermoid | WO2022164316 | FIG. 13B |
| HSP27 BNA | formula (I) | epidermis | carcinoma | | |
| Trastuzumab- | SO1861-linker | Epithelial cells of | Epidermoid | WO2022164316 | FIG. 13B |
| HSP27 BNA | via a | epidermis | carcinoma | | |
| | semicarbazone | | | | |
| | bond linked to | | | | |
| | C-4 of the | | | | |
| | aglycone | | | | |
| ASO for exon | Molecule of | Muscle cells | Duchenne | | |
| skipping in | formula (I) | | muscular dystrophy | | |
| dystrophin | | | | | |
| PMO for exon | Molecule of | Muscle cells | Duchenne | | |
| skipping in | formula (I) | | muscular dystrophy | | |
| dystrophin | | | | | |
| Anti-CD71 mAb - | Molecule of | Muscle cells | Duchenne | | |
| ASO for exon | formula (I) | | muscular dystrophy | | |
| skipping in | | | | | |
| dystrophin | | | | | |
| Anti-CD71 mAb - | Molecule of | Muscle cells | Duchenne | | |
| PMO for exon | formula (I) | | muscular dystrophy | | |
| skipping in | | | | | |
| dystrophin | | | | | |
| PMO for exon | Anti-CD71 | Muscle cells | Duchenne | | |
| skipping in | antibody- | | muscular dystrophy | | |
| dystrophin | SO1861 | | | | |
| PMO for exon | Anti-CD63 | Muscle cells | Duchenne | | |
| skipping in | antibody- | | muscular dystrophy | | |
| dystrophin | SO1861 | | | | |
| PMO for exon | IGF-1 - | Muscle cells | Duchenne | | |
| skipping in | SO1861 | | muscular dystrophy | | |
| dystrophin | conjugate | | | | |
| Anti-CD63 mAb - | SO1861-linker | Muscle cells | Duchenne | | |
| PMO for exon | via a | | muscular dystrophy | | |
| skipping in | semicarbazone | | | | |
| dystrophin | bond linked to | | | | |
| | C-4 of the | | | | |
| | aglycone | | | | |

^{‡}toxin, nucleic acid; effector molecule, effector moiety conjugated with a ligand for binding to an endocytic cell-surface receptor;
^{†}saponin molecule or saponin moiety conjugated with a ligand for binding to an endocytic cell-surface receptor
^{¶}The molecule of formula (I) is depicted in the Definitions section and is also referred to as SO1861-EMCH; molecules according to formula (III), formula 3, formula V are depicted in the Definitions section

Where applicable, Table D above references prior art publications wherein more detailed information can be found, concerning these specific combinations, albeit (as will be understood) without any reference to the general concept underlying the present invention, according to which the saponin component is used/administered with the purpose of extending the duration of effect, extending the dosing interval, reducing the dosing frequency and/or causing a delayed boost in the effect of the effector component. More in particular, in these references the effector component and the saponin component are administered together in the referred in vitro and in vivo models for assessing the stimulatory effect of the saponin on the activity and efficacy of the effector molecule.

### Treatment Regimens

As will be understood by those skilled in the art, based on the present teachings, the methods of treatment provided herein comprise the administration, preferably the intermittent administration, more preferably the repeated intermittent administration of an effector component and a saponin component. Furthermore, it will be understood, based on the present teachings, that the saponin component is advantageously administered some time after the effector component has been administered, e.g. some time after the peak in the pharmacological effect following the administration of the effector component has been reached.

Hence, in preferred embodiments of the invention, a method of treatment as defined herein is provided, wherein the saponin component is administered at least 1 day after the effector component is administered, preferably at least 2 days, at least 3 days, at least 1 week, at least 2 weeks, at least 3 weeks, at least 1 month, at least 2 months, at least 3 months, at least 4 months, at least 5 months, or at least 6 months after the effector component is administered.

In preferred embodiments of the invention, a method of treatment as defined herein is provided, wherein the saponin component is administered during the second, third or fourth quarter of the normal dosing interval of the effector component.

In preferred embodiments of the invention, a method of treatment as defined herein is provided, wherein the saponin component is administered during the second half of the normal dosing interval of the effector component.

In preferred embodiments of the invention, a method of treatment as defined herein is provided, wherein the saponin component is administered during the fourth quarter of the normal dosing interval of the effector component.

In some embodiments of the invention, a method of treatment as defined herein is provided, wherein, during the administration interval of the effector component, the saponin component is administered at least once, such as once, twice, three times or four times, preferably at regular/equal intervals. As will be understood by those skilled in the art, based on the present teachings, said ‘administration interval of the effector component’ may be equal to the normal administration interval typically adhered to in case the treatment does not include administration of the saponin component, but, in accordance with preferred embodiments, it is an administration interval that is extended (compared to said normal interval), in line with what is defined herein elsewhere. Furthermore, it will be understood by those skilled in the art, based on the present teachings, that the term ‘fixed/regular/equal intervals’ is used to denote a regimen wherein the period that elapses between the administration of the effector component and the administration of the saponin component is approximately equal to the period that elapses between the administration of the saponin component and the subsequent administration of effector component. Similarly, in case each treatment cycle involves the administration of the saponin component more than once, regimens are envisaged wherein the period that elapses between the administration of the effector component and the first (subsequent) administration of the saponin component, the period(s) that elapse between each further administration of the saponin component, and the period that elapses between the final administration of the saponin component to the subsequent administration of effector component (i.e. the start of the next cycle), are all approximately equal. Embodiments are also encompassed wherein the respective administration intervals (per cycle) differ. It is envisaged that in such embodiments, the period that elapses between the administration of the effector component and the first administration of the saponin component will typically be longer than the period that elapses between the first administration of the saponin component and each further administration of the saponin component and/or than the period that elapses between the (last) administration of the saponin component and the subsequent administration of effector component (i.e. the start of the next cycle).

For example, an embodiment of the invention concerns a method of treatment as defined herein, wherein the disease is Spinal muscular atrophy and the oligonucleotide therapeutic is selected from the group consisting of nusinersen and therapeutically equivalent ASOs, most preferably nusinersen. Currently, nusinersen is approved for the treatment of Spinal muscular atrophy by administering 4 loading doses on days 0, 14, 28 and 63, followed by maintenance doses once every 4 months thereafter. Embodiments are provided, wherein the treatment comprises the administration of nusinersen maintenance doses at intervals of 5-16 months, e.g. at intervals of least 5 months, at least 6 months, at least 7 months or at least 8 months and/or at intervals of less than 16 months, less than 14 months, less than 12 months, less than 11 months or less than 10 months. In preferred embodiments of the invention, the treatment comprises the administration of the saponin component at least once during each of said nusinersen administration intervals, e.g. once, twice, three time or four times, typically at regular/fixed/equal intervals. In a preferred embodiment of the invention, the method of treatment comprises a maintenance phase comprising repetitive treatment cycles, each treatment cycle starting with the administration of a maintenance dose of nusinersen, followed by the administration of the saponin component, e.g. 2-8 months later or approximately halfway the treatment cycle, whereafter the next cycle starts with the administration of the subsequent nusinersen maintenance dose, where the total period of time that elapses between subsequent nusinersen maintenance doses is at least 5 months, preferably at least 6 months, at least 7 months or at least 8 months. In another preferred embodiment of the invention, the method of treatment comprises a maintenance phase comprising repetitive treatment cycles, each treatment cycle starting with the administration of a maintenance dose of nusinersen, followed by a first administration of the saponin component 2-8 months later, followed by a second administration of the saponin component 2-8 months after the first administration of the saponin component, whereafter the next cycle starts with the administration of the subsequent nusinersen maintenance dose, where the total period of time that elapses between subsequent nusinersen maintenance doses is at least 5 months, preferably at least 6 months, at least 7 months or at least 8 months. As will be understood by those skilled in the art, based on the present teachings, the method of treatment will further typically comprise a nusinersen loading phase in accordance with the currently approved treatment protocol (as reflected above) and the doses of nusinersen will typically be about 12 mg, although embodiments are also envisaged wherein the nusinersen loading and/or maintenance doses can be/are lowered due to the potentiating effect of the saponin component, e.g. to doses within the range of 2-12 mg, such as 4-11 mg or 5-10 mg.

Another exemplary embodiment of the invention concerns a method of treatment as defined herein, wherein the disease is Veno-occlusive disease and the oligonucleotide therapeutic is selected from the group consisting of defibrotide and therapeutically equivalent ASOs, most preferably defibrotide. Currently, defibrotide is approved for the treatment of severe hepatic veno-occlusive disease (VOD) by administering doses every 6 hours for a minimum of 21 days. Embodiments are provided, wherein the treatment comprises the administration of defibrotide doses at intervals of 7-24 hours, e.g. at intervals of least 7 hours, at least 8 hours, at least 9 hours, at least 10 hours, at least 11 hours, at least 12 hours or at least 13 hours and/or at intervals of less than 24 hours, less than 22 hours, less than 20 hours, less than 18 hours, less than 16 hours or less than 14 hours. In preferred embodiments of the invention, the treatment comprises the administration of the saponin component at least once during each of said defibrotide administration intervals, e.g. once, twice, three time or four times, typically at regular/fixed/equal intervals. In a preferred embodiment of the invention, the method of treatment comprises repetitive treatment cycles, each treatment cycle starting with the administration of a dose of defibrotide, followed by the administration of the saponin component, e.g. about 1-12 hours later or approximately halfway the treatment cycle, whereafter the next cycle starts with the administration of the subsequent defibrotide dose, where the total period of time that elapses between subsequent defibrotide doses is at least 7 hours, preferably at least 8 hours, at least 9 hours or at least 10 hours. In another preferred embodiment of the invention, the method of treatment comprises repetitive treatment cycles, each treatment cycle starting with the administration of a dose of defibrotide, followed by a first administration of the saponin component 1-12 hours later, followed by a second administration of the saponin component 1-12 hours after the first administration of the saponin component, whereafter the next cycle starts with the administration of the subsequent defibrotide dose, where the total period of time that elapses between subsequent defibrotide doses is at least 7 hours, preferably at least 8 hours, at least 9 hours or at least 10 hours. As will be understood by those skilled in the art, based on the present teachings, the doses of defibrotide will typically be 6.25 mg/kg body weight, although embodiments are also envisaged wherein the defibrotide doses can be/are lowered due to the potentiating effect of the saponin component, e.g. to doses within the range of 1-6.25 mg/kg body weight, such as 2-5 mg/kg body weight or 3-4 mg/kg body weight.

Another exemplary embodiment of the invention concerns a method of treatment as defined herein, wherein the disease is Hereditary transthyretin-mediated amyloidosis and the oligonucleotide therapeutic is selected from the group consisting of inotersen, and therapeutically equivalent ASOs, most preferably it is inotersen. Currently, inotersen is approved for the treatment of stage 1 or stage 2 polyneuropathy in adult patients with hereditary transthyretin amyloidosis (hATTR) by administering doses once every 7 days. Embodiments are provided, wherein the treatment comprises the administration of notersen doses at intervals of 8-28 days, e.g. at intervals of least 8 days, at least 10 days, at least 12 days or at least 14 days and/or at intervals of less than 28 days, less than 26 days, less than 23 days, less than 20 days or less than 17 days. In preferred embodiments of the invention, the treatment comprises the administration of the saponin component at least once during each of said inotersen administration intervals, e.g. once, twice, three time or four times, typically at regular/fixed/equal intervals. In a preferred embodiment of the invention, the method of treatment comprises repetitive treatment cycles, each treatment cycle starting with the administration of a dose of inotersen, followed by the administration of the saponin component, e.g. 1-14 days later or approximately halfway the treatment cycle, whereafter the next cycle starts with the administration of the subsequent inotersen dose, where the total period of time that elapses between subsequent inotersen doses is at least 8 days, preferably at least 12 days, at least 16 days, at least 20 days, at least 24 days or at least 28 days. In another preferred embodiment of the invention, the method of treatment comprises repetitive treatment cycles, each treatment cycle starting with the administration of a dose of inotersen, followed by a first administration of the saponin component 1-14 days later, followed by a second administration of the saponin component 1-14 days after the first administration of the saponin component, whereafter the next cycle starts with the administration of the subsequent inotersen dose, where the total period of time that elapses between subsequent inotersen doses is at least 8 days, preferably at least 12 days, at least 16 days, at least 20 days, at least 24 days or at least 28 days. As will be understood by those skilled in the art, based on the present teachings, the doses of inotersen will typically be about 284 mg, although embodiments are also envisaged wherein the inotersen doses can be/are lowered due to the potentiating effect of the saponin component, e.g. to doses within the range of 20-284 mg, such as 60-220 mg or 100-160 mg.

Another exemplary embodiment of the invention concerns a method of treatment as defined herein, wherein the disease is Hereditary transthyretin-mediated amyloidosis and the oligonucleotide therapeutic is selected from the group consisting of patisiran and therapeutically equivalent ASOs, most preferably it is patisiran. Currently, patisiran is approved for the treatment of hereditary transthyretin-mediated amyloidosis (hATTR amyloidosis) in adult patients with stage 1 or stage 2 polyneuropathy) by administering doses every 3 weeks. Embodiments are provided, wherein the treatment comprises the administration of patisiran doses at intervals of 4-12 weeks, e.g. at intervals of least 4 weeks, at least 5 weeks, at least 6 weeks, at least 7 weeks or at least 8 weeks, and/or at intervals of less than 12 weeks, less than 11 weeks, less than 10 weeks or less than 9 weeks. In preferred embodiments of the invention, the treatment comprises the administration of the saponin component at least once during each of said patisiran administration intervals, e.g. once, twice, three time or four times, typically at regular/fixed/equal intervals. In a preferred embodiment of the invention, the method of treatment comprises repetitive treatment cycles, each treatment cycle starting with the administration of a dose of patisiran, followed by the administration of the saponin component, e.g. 1-6 weeks later or approximately halfway the treatment cycle, whereafter the next cycle starts with the administration of the subsequent patisiran dose, where the total period of time that elapses between subsequent patisiran doses is at least 4 weeks, preferably at least 5 weeks, at least 6 weeks, at least 8 weeks or at least 10 weeks. In another preferred embodiment of the invention, the method of treatment comprises repetitive treatment cycles, each treatment cycle starting with the administration of a dose of patisiran, followed by a first administration of the saponin component 1-6 weeks later, followed by a second administration of the saponin component 1-6 weeks later after the first administration of the saponin component, whereafter the next cycle starts with the administration of the subsequent patisiran dose, where the total period of time that elapses between subsequent patisiran doses is at least 4 weeks, preferably at least 5 weeks, at least 6 weeks, at least 8 weeks or at least 10 weeks. As will be understood by those skilled in the art, based on the present teachings, the doses of patisiran will typically be 300 μg/kg body weight, although embodiments are also envisaged wherein the patisiran doses can be/are lowered due to the potentiating effect of the saponin component, e.g. to doses within the range of 20-300 μg/kg body weight, such as 50-250 μg/kg body weight or 100-200 μg/kg body weight.

Another exemplary embodiment of the invention concerns a method of treatment as defined herein, wherein the disease is Hereditary transthyretin-mediated amyloidosis and the oligonucleotide therapeutic is selected from the group consisting of vutrisiran and therapeutically equivalent ASOs, most preferably it is vutrisiran. Currently, vutrisiran is approved for the treatment of hereditary transthyretin-mediated amyloidosis (hATTR amyloidosis) in adult patients with stage 1 or stage 2 polyneuropathy.) by administering doses every 3 months. Embodiments are provided, wherein the treatment comprises the administration of vutrisiran doses at intervals of 4-12 months, e.g. at intervals of least 4 months, at least 5 months, at least 6 months, at least 7 months or at least 8 months, and/or at intervals of less than 12 months, less than 11 months, less than 10 months or less than 9 months. In preferred embodiments of the invention, the treatment comprises the administration of the saponin component at least once during each of said vutrisiran administration intervals, e.g. once, twice, three time or four times, typically at regular/fixed/equal intervals. In a preferred embodiment of the invention, the method of treatment comprises repetitive treatment cycles, each treatment cycle starting with the administration of a dose of vutrisiran, followed by the administration of the saponin component, e.g. 1-6 months later or approximately halfway the treatment cycle, whereafter the next cycle starts with the administration of the subsequent vutrisiran dose, where the total period of time that elapses between subsequent vutrisiran doses is at least 4 months, preferably at least 5 months, at least 6 months, at least 8 months or at least 10 months. In another preferred embodiment of the invention, the method of treatment comprises repetitive treatment cycles, each treatment cycle starting with the administration of a dose of vutrisiran, followed by a first administration of the saponin component 1-6 months later, followed by a second administration of the saponin component 1-6 months later after the first administration of the saponin component, whereafter the next cycle starts with the administration of the subsequent vutrisiran dose, where the total period of time that elapses between subsequent vutrisiran doses is at least 4 months, preferably at least 5 months, at least 6 months, at least 8 months or at least 10 months. As will be understood by those skilled in the art, based on the present teachings, the doses of vutrisiran will typically be 25 mg, although embodiments are also envisaged wherein the vutrisiran doses can be/are lowered due to the potentiating effect of the saponin component, e.g. to doses within the range of 1-25 mg, such as 5-20 mg or 10-15 mg.

Another exemplary embodiment of the invention concerns a method of treatment as defined herein, wherein the disease is Acute hepatic Porphyria and the oligonucleotide therapeutic is selected from the group consisting of givosiran and therapeutically equivalent ASOs, most preferably givosiran. Currently, givosiran is approved for the treatment of acute hepatic Porphyria (AHP) in adults and adolescents aged 12 years and older) by administering doses once every month. Embodiments are provided, wherein the treatment comprises the administration of givosiran doses at intervals of 1.5-4 months, e.g. at intervals of least 1.5 months, at least 2 months or at least 2.5 months and/or at intervals of less than 4 months, less than 3.5 months, or less than 3 months. In preferred embodiments of the invention, the treatment comprises the administration of the saponin component at least once during each of said givosiran administration intervals, e.g. once, twice, three time or four times, typically at regular/fixed/equal intervals. In a preferred embodiment of the invention, the method of treatment comprises repetitive treatment cycles, each treatment cycle starting with the administration of a dose of givosiran, followed by the administration of the saponin component 1-8 weeks later, whereafter the next cycle starts with the administration of the subsequent givosiran dose, where the total period of time that elapses between subsequent givosiran doses is at least 1.5 months, preferably at least 2 months, at least 3 months or at least 4 months. In another preferred embodiment of the invention, the method of treatment comprises repetitive treatment cycles, each treatment cycle starting with the administration of a dose of givosiran, followed by a first administration of the saponin component, e.g. 1-8 weeks later or approximately halfway the treatment cycle, followed by a second administration of the saponin component 1-8 weeks after the first administration of the saponin component, whereafter the next cycle starts with the administration of the subsequent givosiran dose, where the total period of time that elapses between subsequent givosiran doses is at least 1.5 months, preferably at least 2 months, at least 3 months or at least 4 months. As will be understood by those skilled in the art, based on the present teachings, the doses of givosiran will typically be about 2.5 mg/kg body weight, although embodiments are also envisaged wherein the givosiran doses can be/are lowered due to the potentiating effect of the saponin component, e.g. to doses within the range of 0.2-2.5 mg/kg body weight, such as 0.5-2.0 mg/kg body weight or 2.0-1.5 mg/kg body weight.

Another exemplary embodiment of the invention concerns a method of treatment as defined herein, wherein the disease is Primary hyperoxaluria type 1 and the oligonucleotide therapeutic is selected from the group consisting of lumasiran and therapeutically equivalent ASOs, most preferably lumasiran. Currently, lumasiran is approved for the treatment of primary hyperoxaluria type 1 (PHI1) in all age groups by administering loading doses once a month for 3 months, followed by maintenance doses once every 3 months thereafter. Embodiments are provided, wherein the treatment comprises the administration of lumasiran maintenance doses at intervals of 4-15 months, e.g. at intervals of least 4 months, at least 5 months, at least 6 months or at least 7 months and/or at intervals of less than 15 months, less than 13 months, less than 11 months, less than 10 months or less than 9 months. In preferred embodiments of the invention, the treatment comprises the administration of the saponin component at least once during each of said lumasiran administration intervals, e.g. once, twice, three time or four times, typically at regular/fixed/equal intervals. In a preferred embodiment of the invention, the method of treatment comprises a maintenance phase comprising repetitive treatment cycles, each treatment cycle starting with the administration of a maintenance dose of lumasiran, followed by the administration of the saponin component, e.g. 1-6 months later or approximately halfway the treatment cycle, whereafter the next cycle starts with the administration of the subsequent lumasiran maintenance dose, where the total period of time that elapses between subsequent lumasiran maintenance doses is at least 4 months, preferably at least 5 months, at least 6 months or at least 7 months. In another preferred embodiment of the invention, the method of treatment comprises a maintenance phase comprising repetitive treatment cycles, each treatment cycle starting with the administration of a maintenance dose of lumasiran, followed by a first administration of the saponin component 1-6 months later, followed by a second administration of the saponin component 1-6 months after the first administration of the saponin component, whereafter the next cycle starts with the administration of the subsequent lumasiran maintenance dose, where the total period of time that elapses between subsequent lumasiran maintenance doses is at least at least 4 months, preferably at least 5 months, at least 6 months or at least 7 months. As will be understood by those skilled in the art, based on the present teachings, the method of treatment will further typically comprise a lumasiran loading phase in accordance with the currently approved treatment protocol (as reflected above) and the doses of lumasiran will typically be about 3 mg/kg body weight, although embodiments are also envisaged wherein the lumasiran loading and/or maintenance doses can be/are lowered due to the potentiating effect of the saponin component, e.g. to doses within the range of 0.5-3 mg/kg body weight, such as 1-2.5 mg/kg body weight or 1.5-2 mg/kg body weight.

Another exemplary embodiment of the invention concerns a method of treatment as defined herein, wherein the disease is Primary hypercholesterolemia and the oligonucleotide therapeutic is selected from the group consisting of inclisiran and therapeutically equivalent ASOs, most preferably inclisiran. Currently, inclisiran is approved for the treatment of primary hypercholesterolaemia (heterozygous familial and non-familial) or mixed dyslipidaemia by administering 2 loading doses once every 3 months, followed by maintenance doses once every 6 months thereafter. Embodiments are provided, wherein the treatment comprises the administration of inclisiran maintenance doses at intervals of 7-24 months, e.g. at intervals of least 8 months, at least 9 months, at least 10 months or at least 12 months and/or at intervals of less than 23 months, less than 21 months, less than 19 months, less than 17 months or less than 15 months. In preferred embodiments of the invention, the treatment comprises the administration of the saponin component at least once during each of said inclisiran administration intervals, e.g. once, twice, three time or four times, typically at regular/fixed/equal intervals. In a preferred embodiment of the invention, the method of treatment comprises a maintenance phase comprising repetitive treatment cycles, each treatment cycle starting with the administration of a maintenance dose of inclisiran, followed by the administration of the saponin component, e.g. 1-12 months later or approximately halfway the treatment cycle, whereafter the next cycle starts with the administration of the subsequent inclisiran maintenance dose, where the total period of time that elapses between subsequent inclisiran maintenance doses is at least 7 months, preferably at least 8 months, at least 9 months or at least 10 months. In another preferred embodiment of the invention, the method of treatment comprises a maintenance phase comprising repetitive treatment cycles, each treatment cycle starting with the administration of a maintenance dose of inclisiran, followed by a first administration of the saponin component 1-12 months later, followed by a second administration of the saponin component 1-12 months after the first administration of the saponin component, whereafter the next cycle starts with the administration of the subsequent inclisiran maintenance dose, where the total period of time that elapses between subsequent inclisiran maintenance doses is at least 7 months, preferably at least 8 months, at least 9 months or at least 10 months. As will be understood by those skilled in the art, based on the present teachings, the method of treatment will further typically comprise a inclisiran loading phase in accordance with the currently approved treatment protocol (as reflected above) and the doses of inclisiran will typically be about 284 mg, although embodiments are also envisaged wherein the inclisiran loading and/or maintenance doses can be/are lowered due to the potentiating effect of the saponin component, e.g. to doses within the range of 20-284 mg, such as 50-250 mg or 100-200 mg.

Another exemplary embodiment of the invention concerns a method of treatment as defined herein, wherein the disease is Neovascular (Wet) Age-Related Macular Degeneration and the oligonucleotide therapeutic is selected from the group consisting of pegaptanib and therapeutically equivalent ASOs, most preferably pegaptanib. Currently, pegaptanib is approved for the treatment of acute hepatic Porphyria (AHP) in adults and adolescents aged 12 years and older by administering doses once every 6 weeks (9 injections per year). Embodiments are provided, wherein the treatment comprises the administration of pegaptanib doses at intervals of 7-24 weeks, e.g. at intervals of least 7 weeks, at least 8 weeks, at least 10 weeks, at least 12 weeks or at least 14 weeks and/or at intervals of less than 24 weeks, less than 22 weeks, less than 20 weeks, less than 18 weeks, or less than 16 weeks. In preferred embodiments of the invention, the treatment comprises the administration of the saponin component at least once during each of said pegaptanib administration intervals, e.g. once, twice, three time or four times, typically at regular/fixed/equal intervals. In a preferred embodiment of the invention, the method of treatment comprises repetitive treatment cycles, each treatment cycle starting with the administration of a dose of pegaptanib, followed by the administration of the saponin component, e.g. 1-12 weeks later or approximately halfway the treatment cycle, whereafter the next cycle starts with the administration of the subsequent pegaptanib dose, where the total period of time that elapses between subsequent pegaptanib doses is at least 7 weeks, preferably at least 8 weeks, at least 10 weeks, at least 14 weeks or at least 18 weeks. In another preferred embodiment of the invention, the method of treatment comprises repetitive treatment cycles, each treatment cycle starting with the administration of a dose of pegaptanib, followed by a first administration of the saponin component 1-12 weeks later, followed by a second administration of the saponin component 1-12 weeks after the first administration of the saponin component, whereafter the next cycle starts with the administration of the subsequent pegaptanib dose, where the total period of time that elapses between subsequent pegaptanib doses is at least at least 8 weeks, at least 10 weeks, at least 14 weeks or at least 18 weeks. As will be understood by those skilled in the art, based on the present teachings, the doses of pegaptanib will typically be about 1.65 mg, although embodiments are also envisaged wherein the pegaptanib doses can be/are lowered due to the potentiating effect of the saponin component, e.g. to doses within the range of 0.2-1.65 mg, such as 0.5-1.5 mg or 0.8-1.2 mg.

Another exemplary embodiment of the invention concerns a method of treatment as defined herein, wherein the disease is Familial chylomicronemia syndrome and the oligonucleotide therapeutic is selected from the group consisting of volanesorsen and therapeutically equivalent ASOs, most preferably volanesorsen. Currently, volanesorsen is approved for the treatment of patients with genetically confirmed familial chylomicronemia syndrome (FCS) and at high risk for pancreatitis, in whom response to diet and triglyceride lowering therapy has been inadequate by administering loading doses once weekly for 3 months, followed by maintenance doses once every 2 weeks thereafter. Embodiments are provided, wherein the treatment comprises the administration of volanesorsen maintenance doses at intervals of 3-8 weeks, e.g. at intervals of least 3 weeks, at least 4 weeks or at least 5 weeks and/or at intervals of less than 8 weeks, less than 7 weeks or less than 6 weeks. In preferred embodiments of the invention, the treatment comprises the administration of the saponin component at least once during each of said volanesorsen administration intervals, e.g. once, twice, three time or four times, typically at regular/fixed/equal intervals. In a preferred embodiment of the invention, the method of treatment comprises a maintenance phase comprising repetitive treatment cycles, each treatment cycle starting with the administration of a maintenance dose of volanesorsen, followed by the administration of the saponin component, e.g. 1-4 weeks later or approximately halfway the treatment cycle, whereafter the next cycle starts with the administration of the subsequent volanesorsen maintenance dose, where the total period of time that elapses between subsequent volanesorsen maintenance doses is at least 3 weeks, preferably at least 4 weeks, at least 5 weeks or at least 6 weeks. In another preferred embodiment of the invention, the method of treatment comprises a maintenance phase comprising repetitive treatment cycles, each treatment cycle starting with the administration of a maintenance dose of volanesorsen, followed by a first administration of the saponin component 1-4 weeks later, followed by a second administration of the saponin component 1-4 weeks after the first administration of the saponin component, whereafter the next cycle starts with the administration of the subsequent volanesorsen maintenance dose, where the total period of time that elapses between subsequent volanesorsen maintenance doses is at least 3 weeks, preferably at least 4 weeks, at least 5 weeks or at least 6 weeks. As will be understood by those skilled in the art, based on the present teachings, the method of treatment will further typically comprise a volanesorsen loading phase in accordance with the currently approved treatment protocol (as reflected above) and the doses of volanesorsen will typically be about 285 mg per eye, although embodiments are also envisaged wherein the volanesorsen loading and/or maintenance doses can be/are lowered due to the potentiating effect of the saponin component, e.g. to doses within the range of 10-285 mg, such as 50-250 mg or 100-200 mg.

Another exemplary embodiment of the invention concerns a method of treatment as defined herein, wherein the disease is Cytomegalovirus retinitis and the oligonucleotide therapeutic is selected from the group consisting of fomivirsen and therapeutically equivalent ASOs, most preferably fomivirsen. Currently, fomivirsen is approved for the treatment of Cytomegalovirus retinitis in patients with acquired immunodeficiency syndrome (AIDS) by administering 3 loading doses once every week, followed by maintenance doses once every 2 weeks thereafter (for newly diagnosed patients). Embodiments are provided, wherein the treatment comprises the administration of fomivirsen maintenance doses at intervals of 3-16 weeks, e.g. at intervals of least 3 weeks, at least 4 weeks, at least 5 weeks, at least 6 weeks or at least 7 weeks and/or at intervals of less than 14 weeks, less than 12 weeks, less than 11 weeks, less than 10 weeks, less than 9 weeks or less than 8 weeks. In preferred embodiments of the invention, the treatment comprises the administration of the saponin component at least once during each of said fomivirsen administration intervals, e.g. once, twice, three time or four times, typically at regular/fixed/equal intervals. In a preferred embodiment of the invention, the method of treatment comprises a maintenance phase comprising repetitive treatment cycles, each treatment cycle starting with the administration of a maintenance dose of fomivirsen, followed by the administration of the saponin component, e.g. 1-4 weeks later or approximately halfway the treatment cycle, whereafter the next cycle starts with the administration of the subsequent fomivirsen maintenance dose, where the total period of time that elapses between subsequent fomivirsen maintenance doses is at least 3 weeks, preferably at least 4 weeks, at least 5 weeks or at least 6 weeks. In another preferred embodiment of the invention, the method of treatment comprises a maintenance phase comprising repetitive treatment cycles, each treatment cycle starting with the administration of a maintenance dose of fomivirsen, followed by a first administration of the saponin component 1-4 weeks later, followed by a second administration of the saponin component 1-4 weeks after the first administration of the saponin component, whereafter the next cycle starts with the administration of the subsequent fomivirsen maintenance dose, where the total period of time that elapses between subsequent fomivirsen maintenance doses is at least 3 weeks, preferably at least 4 weeks, at least 5 weeks or at least 6 weeks. As will be understood by those skilled in the art, based on the present teachings, the method of treatment will further typically comprise a fomivirsen loading phase in accordance with the currently approved treatment protocol (as reflected above) and the doses of fomivirsen will typically be about 165 μg per eye, although embodiments are also envisaged wherein the fomivirsen loading and/or maintenance doses can be/are lowered due to the potentiating effect of the saponin component, e.g. to doses within the range of 10-165 μg, such as 25-150 μg or 40-135 μg.

Another exemplary embodiment of the invention concerns a method of treatment as defined herein, wherein the disease is hemophilia A or B, and/or (other) SERPINC1-related diseases and the oligonucleotide therapeutic is selected from the group consisting of fitusiran and therapeutically equivalent double and single stranded oligonucleotides, most preferably it is fitusiran. Currently, fitusiran is in advanced Phase 3 clinical trials as a prophylactic therapy for individuals with hemophilia A or B by AT (antithrombin) lowering in individuals with hemophilia to increase thrombin generation, leading to enhanced hemostasis. In the phase 3 clinical trials, participants received a 80 mg sc dose of fitusiran once a month (for a total period of 7 months).

Embodiments are provided, wherein the treatment comprises the administration of fitusiran doses at intervals of 1-12 months, e.g. at intervals of least 1.5 months, at least 2 months, at least 2.5 months, at least 3 months, at least 3.5 months, at least 4 months, at least 5 months or at least 6 months and/or at intervals of less than 12 months, less than 11 months, less than 10 months or less than 9 months. In preferred embodiments of the invention, the treatment comprises the administration of the saponin component at least once during each fitusiran administration interval, e.g. once, twice, three times or four times, typically at regular/fixed/equal intervals. In a preferred embodiment of the invention, the method of treatment comprises repetitive treatment cycles, each treatment cycle starting with the administration of a dose of fitusiran, followed by the administration of the saponin component, e.g., 2 weeks to 3 months later or approximately halfway the treatment cycle, whereafter the next cycle starts with the administration of the subsequent fitusiran dose, where the total period of time that elapses between subsequent fitusiran doses is at least 1.5 month, preferably at least 2 months, at least 2.5 months, at least 3 months, at least 3.5 months, at least 4 months, at least 5 months or at least 6 months. In another preferred embodiment of the invention, the method of treatment comprises repetitive treatment cycles, each treatment cycle starting with the administration of a dose of fitusiran, followed by a first administration of the saponin component, e.g., 2 weeks to 3 months later, followed by a second administration of the saponin component 2 weeks to 3 months later after the first administration of the saponin component, whereafter the next cycle starts with the administration of the subsequent fitusiran dose, where the total period of time that elapses between subsequent fitusiran doses is at least 2 months, preferably at least 2.5 months, at least 3.5 months, at least 4 months, at least 6 months, at least 7 months, at least 8 months or at least 9 months. As will be understood by those skilled in the art, based on the present teachings, the initial doses of fitusiran will typically be around 80 mg, although embodiments are also envisaged wherein the fitusiran doses can be/are lowered due to the potentiating effect of the saponin component, e.g. to doses within the range of 1-60 mg, such as 2.5-40 mg or 5-25 mg.

Furthermore, in preferred embodiments of the invention, a method of treatment as defined herein is provided, wherein the saponin component is administered at a dose of at least 0.005 mg/kg, such as at least 0.01 mg/kg, at least 0.025, at least 0.05 mg/kg or at least 0.075 mg/kg and/or at a dose of less than 2 mg/kg, such as at a dose of less than 1 mg/kg, less than 0.5 mg/kg or less than 0.25 mg/k, most preferably at a dose of about 0.1 mg/kg.

Furthermore, in preferred embodiments of the invention, a method of treatment as defined herein is provided, wherein the saponin component is administered at a dose of at least 0.001 mg/kg, such as at least 0.005 mg/kg, at least 0.01, at least 0.02 mg/kg or at least 0.025 mg/kg and/or at a dose of less than 1 mg/kg, such as at a dose of less than 0.5 mg/kg, less than 0.1 mg/kg or less than 0.05 mg/k, most preferably at a dose of about 0.03 mg/kg.

### EXAMPLES

### Abbreviations

Ab Antibody
AH Acylhydrazone bond
AEM N-(2-Aminoethyl)maleimide trifluoroacetate salt
AMPD 2-Amino-2-methyl-1,3-propanediol
BOP (Benzotriazol-1-yloxy)tris(dimethylamino)phosphonium hexafluorophosphate
Cet Cetuximab
Cy5 Cyanine5 dye
DIPEA N,N-diisopropylethylamine
DMF N,N-dimethylformamide
DTT Dithiothreitol
EDCl·HCl 3-((Ethylimino)methyleneamino)-N,N-dimethylpropan-1-aminium chloride
EMCH.TFA N-(ϵ-maleimidocaproic acid) hydrazide, trifluoroacetic acid salt
GalNAc N-Acetylgalactosamine
GN3 trimeric GalNAc also referred to as trivalent GalNAc
HATU 1-[Bis(dimethylamino)methylene]-1H-1,2,3-triazolo[4,5-b]pyridinium 3-oxid hexafluorophosphate
min minutes
NEM N-Ethylmaleimide
NMM 4-Methylmorpholine
r.t. retention time
SC Semicarbazone bond
SH Thiol
siTTR siRNA targeting murine transthyretin
TCEP tris(2-carboxyethyl)phosphine hydrochloride
Temp temperature
TFA trifluoroacetic acid
TTR Transthyretin

### Materials

| | | Cat. Code/ | |
|---|---|---|---|
| Description | Supplier | Product No. | Lot No. |
| Cetuximab | Merck Europe | Erbitux | |
| Sulfo-Cy5-NHS | Lumiprobe | 63320 | BCV7C |
| Goat anti-Human Kappa - | Southern Biotech | 2060-05 | C4119-VG59B |
| HRP | | | |
| Goat anti-Human IgG - | Southern Biotech | 2040-05 | B3919-XD29C |
| HRP | | | |
| “presentation buffer” | Fleet BioScience | SOP032 Buffer 113 | BF3371 |
| (Dulbecco's PBS pH 7.5) | Ltd | | |
| Dulbecco's PBS pH 7.5, | Fleet BioScience | — | DV254/12-DPBST |
| 0.01% polysorbate 80 | Ltd | | |
| (DPBST pH 7.5) | | | |
| “SEC analysis buffer” | Fleet BioScience | SOP032 Buffer 114 | BF3385 |
| (DPBS:IPA 85:15) | Ltd | | |
| PBS, 0.05% Tween 20 | Fleet BioScience | — | DV238-173 |
| | Ltd | | |
| MOPS running buffer | Life Technologies | NP0001 | 2160459 |
| LDS sample buffer | Life Technologies | NP0007 | 2152677A |
| NuPAGE Transfer Buffer | Thermo | NP0006 | 2148502 |
| (20X) | | | |
| Blocking Buffer | Thermo | 37537 | UK292772 |
| Methanol | VWR | 20847 | 18G164024 |
| DTT | Sigma | 43815 | BCBS8122V |
| Polysorbate (Tween) 80 | Sigma | P1754 | BCBR2510V |
| Glycine | VWR | 104201 | V01604600825 |
| Zeba 10 ml spin desalting | Thermo | 89893 | UF283301 |
| column | | | |
| PD10 G25 | GE | 17085101 | 17046497 |
| Float-a-lyser G2 | Sigma | G235065 | 3317420 |
| Vivaspin T4 | Sartorius | VS04T01 | 193600057 |
| Vivaspin T15 | Sartorius | VS15T02 | 1803004VS |
| 0.2 μm Filter | Sartorius | 17761 | 91500103 |
| BCA Assay kit | Thermo | 23225 | UF281362A |
| BGG standard | Thermo | 23212 | TK273657 |
| Novex protein standards | Life Technologies | LC5800 | 2154538 |
| ladder | | | |
| 4-12% BT SDS-PAGE gel | Life Technologies | NP0323BOX | 19120670 |
| PAGE Blue protein stain | Thermo | 24620 | ON643585 |
| NuPAGE Antioxidant | Thermo | NP0005 | 2171517 |
| Nitrocellulose membrane | Thermo | LC20000 | 1708367 |
| Western Blotting filter | Thermo | 88600 | QA1889445 |
| paper | | | |
| CN/DAB Substrate (10X) | Thermo | 1855900 | UG287470 |
| Stable Peroxide Substrate | Thermo | 1855901 | UF285529 |
| Buffer | | | |
| TLC silica gel 60 | Merck | 1055540001 | HX957486 |
| Biosep s3000 aSEC | Phenomenex | SEC-s3000 | CLM0044 |
| column | | | |

### Analytical Methods

LC-MS method 1

Apparatus: Waters lClass; Bin. Pump: UPIBSM, SM: UPISMFTN with SO; UPCMA, PDA: UPPDATC, 210-320 nm, SQD: ACQ-SQD2 ESI, mass ranges depending on the molecular weight of the product: neg or neg/pos within in a range of 1500-2400 or 2000-3000; ELSD: gas pressure 40 psi, drift tube temp: 50° C.; column: Acquity C18, 50×2.1 mm, 1.7 μm Temp: 60° C., Flow: 0.6 mL/min, Iin. Gradient depending on the polarity of the product:
^{A}t₀=2% A, t_{5.0 min}=50% A, t_{6.0 min}=98% A
^{B}t₀=2% A, t_{5.0 min}=98% A, t_{6.0 min}=98% A
Posttime: 1.0 min, Eluent A: acetonitrile, Eluent B: 10 mM ammonium bicarbonate in water (pH=9.5).

### LC-MS Method 2

Apparatus: Waters IClass; Bin. Pump: UPIBSM, SM: UPISMFTN with SO; UPCMA, PDA: UPPDATC, 210-320 nm, SQD: ACQ-SQD2 ESI, mass ranges depending on the molecular weight of the product: pos/neg 100-800 or neg 2000-3000; ELSD: gas pressure 40 psi, drift tube temp: 50° C.; column: Waters XSelect™ CSH C18, 50×2.1 mm, 2.5 μm, Temp: 25° C., Flow: 0.5 mL/min, Gradient: t_{0 min}=5% A, t_{2.0 min}=98% A, t_{2.7 min}=98% A, Posttime: 0.3 min, Eluent A: acetonitrile, Eluent B: 10 mM ammonium bicarbonate in water (pH=9.5).

LC-MS meqthod 3

Apparatus: Waters IClass; Bin. Pump: UPIBSM, SM: UPISMFTN with SO; UPCMA, PDA: UPPDATC, 210-320 nm, SQD: ACQ-SQD2 ESI, mass ranges depending on the molecular weight of the product pos/neg 105-800, 500-1200 or 1500-2500; ELSD: gas pressure 40 psi, drift tube temp: 50° C.; column: Waters XSelect™ CSH C18, 50×2.1 mm, 2.5 μm, Temp: 40° C., Flow: 0.5 mL/min, Gradient: t_{0 min}=5% A, t_{2.0 min}=98% A, t_{2.7 min}=98% A, Posttime: 0.3 min, Eluent A: 0.1% formic acid in acetonitrile, Eluent B: 0.1% formic acid in water.

### LC-MS Method 4

Apparatus: Waters IClass; Bin. Pump: UPIBSM, SM: UPISMFTN with SO; UPCMA, PDA: UPPDATC, 210-320 nm, SQD: ACQ-SQD2 ESI, mass ranges depending on the molecular weight of the product: pos/neg 100-800 or neg 2000-3000; ELSD: gas pressure 40 psi, drift tube temp: 50° C. column: Waters Acquity Shield RP18, 50×2.1 mm, 1.7 μm, Temp: 25° C., Flow: 0.5 mL/min, Gradient: t_{0 min}=5% A, t_{2.0 min}=98% A, t_{2.7 min}=98% A, Posttime: 0.3 min, Eluent A: acetonitrile, Eluent B: 10 mM ammonium bicarbonate in water (pH=9.5).

LC-MS methqod 5

Apparatus: Waters IClass; Bin. Pump: UPIBSM, SM: UPISMFTN with SO; UPCMA, PDA: UPPDATC, 210-320 nm, SQD: ACQ-SQD2 ESI, mass ranges depending on the molecular weight of the product: neg/pos within in a range of 1500-2700; ELSD: gas pressure 40 psi, drift tube temp: 50° C.; column: Acquity Premier Peptide BEH C18, 50×2.1 mm, 1.7 μm Temp: 25° C., Flow: 0.45 mL/min, Gradient depending on the polarity of the product:
^{A}t₀=2% B, t_{4.0 min}=50% B, t_{5.0 min}=98% B, t_{6.0 min}=98% B
^{B}t₀=5% B, t_{6.0 min}=98% B, t_{6.0 min}=98% B
, Posttime: 1.0 min, Eluent A: 10 mM ammonium bicarbonate in water (pH=9.5), Eluent B: acetonitrile.

### Preparative Methods

### Preparative MP-LC Method 1

Instrument type: Reveleris™ prep MPLC; column: Waters XSelect™ CSH C18 (145×25 mm, 10 μm); Flow: 40 mL/min; Column temp: room temperature; Eluent A: 10 mM ammoniumbicarbonate in water pH=9.0); Eluent B: 99% acetonitrile+1% 10 mM ammoniumbicarbonate in water; Gradient:
^{A}t_{0 min}=5% B, t_{1 min}=5% B, t_{2 min}=10% B, t_{17 min}=50% B, t_{18 min}=100% B, t_{23 min}=100% B
^{A}t_{0 min}=5% B, t_{1 min}=5% B, t_{2 min}=20% B, t_{17 min}=60% B, t_{18 min}=100% B, t_{23 min}=100% B; Detection UV: 210, 235, 254 nm and ELSD.
   Preparative MP-LC method 2

Instrument type: Reveleris™ prep MPLC; Column: Phenomenex LUNA C18(3) (150×25 mm, 10 μm);

Flow: 40 mL/min; Column temp: room temperature; Eluent A: 0.1% (v/v) Formic acid in water, Eluent B: 0.1% (v/v) Formic acid in acetonitrile; Gradient:
^{A}t_{0 min}=5% B, t_{1 min}=5% B, t_{2 min}=20% B, t_{17 min}=60% B, t_{18 min}=100% B, t_{23 min}=100% B ^{B}t_{0 min}=2% B, t_{1 min}=2% B, t_{2 min}=2% B, t_{17 min}=30% B, t_{18 min}=100% B, t_{23 min}=100% B
^{C}t_{0 min}=5% B, t_{1 min}=5% B, t_{2 min}=10% B, t_{17 min}=50% B, t_{18 min}=100% B, t_{23 min}=100% B
^{D}t_{0 min}=5% B, t_{1 min}=5% B, t_{2 min}=5% B, t_{17 min}=40% B, t_{18 min}=100% B, t_{23 min}=100% B
; Detection UV: 210, 235, 254 nm and ELSD.

### Preparative LC-MS Method 3

MS instrument type: Agilent Technologies G6130B Quadrupole; HPLC instrument type: Agilent Technologies 1290 preparative LC; Column: Waters XSelect™ CSH (C18, 150×19 mm, 10 μm); Flow: 25 ml/min; Column temp: room temperature; Eluent A: 100% acetonitrile; Eluent B: 10 mM ammonium bicarbonate in water pH=9.0; Gradient:
^{A}t₀=20% A, t_{2.5 min}=20% A, t_{11 min}=60% A, t_{13 min}=100% A, t_{17 min}=100% A
^{B}t₀=5% A, t_{2.5 min}=5% A, t_{11 min}=40% A, t_{13 min}=100% A, t_{17 min}=100% A
; Detection: DAD (210 nm); Detection: MSD (ESI pos/neg) mass range: 100-800; Fraction collection based on DAD.

### Preparative LC-MS Method 4

MS instrument type: Agilent Technologies G6130B Quadrupole; HPLC instrument type: Agilent Technologies 1290 preparative LC; Column: Waters XBridge Protein (C4, 150×19 mm, 10 μm); Flow: 25 ml/min; Column temp: room temperature; Eluent A: 100% acetonitrile; Eluent B: 10 mM ammonium bicarbonate in water pH=9.0; Gradient:
^{A}t₀=2% A, t_{2.5 min}=2% A, t_{11 min}=30% A, t_{13 min}=100% A, t_{17 min}=100% A
^{B}t₀=10% A, t_{2.5 min}=10% A, t_{1 min}=50% A, t_{13 min}=100% A, t_{17 min}=100% A
^{C}t₀=5% A, t_{2.5 min}=5% A, t_{1 min}=40% A, t_{13 min}=100% A, t_{17 min}=100% A
; Detection: DAD (210 nm); Detection: MSD (ESI pos/neg) mass range: 100-800; Fraction collection based on DAD

### Flash Chromatography

Grace Reveleris X2® C-815 Flash; Solvent delivery system: 3-piston pump with auto-priming, 4 independent channels with up to 4 solvents in a single run, auto-switches lines when solvent depletes; maximum pump flow rate 250 mL/min; maximum pressure 50bar (725 psi); Detection: UV 200-400 nm, combination of up to 4 UV signals and scan of entire UV range, ELSD; Column sizes: 4-330 g on instrument, luer type, 750 g up to 3000 g with optional holder.

### UV-Vis Spectrophotometry

Antibody concentrations, and Sulfo-Cy5 concentrations and incorporations were determined using a Thermo Nanodrop 2000 spectrometer. Antibody concentrations in the conjugates were determined by BCA assay. BCA assays were conducted using a Thermo SkanlT plate reader.
Sulfo-Cy5; mass E646=355.7 M−1 cm-1, Rz (280:646)=0.04.
Ellmans (TNB) ε 412=14,150 M−1 cm-1
Cetuximab E 280=1.4 (mg/ml)-1 cm-1
Cetuximab-SO1861; mass E280=1.4 (mg/ml)-1 cm-1

### Thin Layer Chromatography

˜8×8 cm TLC cards were cut and Sulfo-Cy5 (1:100) and STB28/1-1 to 5 and 2-1 to 5 (1:1) spotted (0.5 μl) 6 mm apart and allowed to dry. The TLC was run with methanol as mobile phase and inspected visually and under short/long-wave UV. For ‘quantitative’ measurement of residual free Sulfo-Cy5, Sulfo-Cy5 standards (10,000, 1,000, 100 and 0 ng/ml) were ran by TLC and analysed by Fluorescence spectrometry alongside the residual free Sulfo-Cy5 in conjugate samples.

### Fluorescence Spectrometry

TLC plates were analysed using a Perkin Elmer LS55 Fluorescence Spectrometer with plate reader attachment, in TLC reader mode (Aex=646 nm; Aem=720 nm; slit widths=10 nm). Quantities of residual free Sulfo-Cy5 were estimated with respect to Sulfo-Cy5 standards, discounting differences in spot sizes/shapes.

### SEC

Native antibody and conjugates were analysed by SEC using an Akta purifier 100 system and Biosep SEC-s3000 column eluting with DPBS:IPA (85:15). % purity was determined by integration of the antibody peak with respect to trace aggregate peaks.

### SDS-PAGE and Western Blotting

Native antibody and conjugates were analysed under heat denaturing non-reducing and reducing conditions by SDS-PAGE against a protein ladder using a 4-12% bis-tris gel and MOPS as running buffer (200V, 40 minutes). Samples were prepared to 0.5 mg/ml, comprising LDS sample buffer and MOPS running buffer as diluent. For reducing samples, DTT was added to a final concentration of 50 mM.

Samples were heat treated for 2 minutes at 90-95° C. and 5 μg (10 μl) added to each well. Protein ladder (10 μl) was loaded without pre-treatment. Empty lines were filled with 1×LDS sample buffer (10 μl). After the gel was run, it was washed thrice with DI water (100 ml) with shaking (15 minutes, 200 rpm). Coomassie staining was performed by shaker-incubating the gel with PAGEBlue protein stain (30 ml) (60 minutes, 200 rpm). Excess staining solution was removed, rinsed twice with DI water (100 ml) and destained with DI water (100 ml) (60 minutes, 200 rpm). The resulting gel was imaged and processed using imageJ.

For Western Blotting, washed gel (not Coomassie stained) was transferred to nitrocellulose membrane using the X-Cell blot module with the following setup (BP-BP-FP-Gel-NC-FP-BP-FP-Gel-NC-FP-BP-BP) and conditions (30V, 0.17 Amps, 60 minutes) and freshly prepared transfer buffer. BP—blotting pad; FP—Filter pad; NC—Nitrocellulose membrane. After, the NC were washed thrice with PBS-T (100 ml), non-specific sites blocked with blocking buffer (30 ml) with shaking (10 minutes, 200 rpm) then active sites labelled with a combination of Goat anti-Human Kappa-HRP (1:2000) and Goat anti-Human IgG-HRP (1:2000) (30 ml) diluted in blocking buffer with shaking (60 minutes, 200 rpm). After, the NC were washed with PBS-T (100 ml) and complexed antibody detected with CN/DAB substrate (25 ml) freshly prepared using stable peroxide substrate buffer. Colour development was observed visually and the resulting NC photographed.

### SO1861-AH-Maleimide

SO1861-AH-Maleimide (also referred to as SO1861-AH-Mal or SO1861-EMCH) was produced as previously described in WO 2021/259507A1 (page 72, Example 3, referred to as “SO1861-EMCH synthesis”).. To SO1861 (121 mg, 0.065 mmol) and EMCH.TFA (110 mg, 0.325 mmol) was added methanol (extra dry, 3.00 mL) and TFA (0.020 mL, 0.260 mmol). The reaction mixture stirred at room temperature. After 1.5 hours the reaction mixture was subjected to preparative MP-LC.1 Fractions corresponding to the product were immediately pooled together, frozen and lyophilized overnight to give the title compound (120 mg, 90%) as a white fluffy solid. Purity based on LC-MS 96%.

LRMS (m/z): 2069 [M−1]1−

LC-MS r.t. (min): 1.084

See FIG. 8 for the formula of SO1861-AH-azide (referred to as ‘SO1861-AH-N₃’ in FIG. 8).

### SO1861-AH-Maleimide-Block (Saponin Molecule According to Formula (V), Also Referred to as SO1861-AH-Block and SO1861-Ald-EMCH-Mercaptoethanol)

To SO1861-AH-Maleimide (0.1 mg, 48 nmol) 200 μL mercaptoethanol (18 mg, 230 μmol) was added and the solution was shaken for 1 h at 800 rpm and room temperature on a ThermoMixer C (Eppendorf). After shaking for 1 h, the solution was diluted with methanol and dialyzed extensively for 4 h against methanol using regenerated cellulose membrane tubes (Spectra/Por 7) with a MWCO of 1 kDa. After dialysis the SO1861-Ald-EMCH-mercaptoethanol was provided (saponin molecule according to formula (V)), an aliquot was taken out and analyzed via MALDI-TOF-MS. (RP mode): m/z 2193 Da ([M+K]+, SO1861-AH-Block), m/z 2185 Da ([M+K]+, SO1861-AH-Block), m/z 2170 Da ([M+Na]+, SO1861-AH-Block).

### SO1861-AH-azide

To SO1861 (60 mg, 0.032 mmol) and 1-azido-3,6,9,12-tetraoxapentadecane-15-hydrazide (39.3 mg, 0.129 mmol) was added methanol (extra dry, 1.00 mL) and TFA (9.86 μl, 0.129 mmol) and the reaction mixture was shaken for 1 min and left standing at room temperature. After 2 hours the reaction mixture was subjected to preparative MP-LC.¹ Fractions corresponding to the product were immediately pooled together, frozen and lyophilized overnight to give the title compound (58.4 mg, 84%) as a white fluffy solid. Purity based on LC-MS 100%.

LRMS (m/z): 2150 [M−1]¹⁻

LC-MS r.t. (min): 10.10^{3B}

### SO1861-SC-azide Synthesis

### Intermediate 1:

### tert-butyl 2-(4-(6-azidohexanoyl)piperazine-1-carbonyl)hydrazine-1-carboxylate

6-azidohexanoic acid (603 mg, 3.84 mmol), tert-butyl 2-(piperazine-1-carbonyl)hydrazine-1-carboxylate (781 mg, 3.20 mmol), EDCl·HCl (735 mg, 3.84 mmol) and Oxyma Pure (591 mg, 4.16 mmol) were dissolved in a mixture of dichloromethane (25 mL) and DIPEA (835 μL, 4.80 mmol) and the reaction mixture was stirred at room temperature. After 2 hours the reaction mixture was evaporated in vacuo and the residue was dissolved in ethyl acetate (50 mL). The resulting solution was washed with 0.5 N potassium bisulphate solution (50 mL), saturated sodium bicarbonate solution (2×50 mL) and brine (50 mL), dried over Na₂SO₄, filtered and evaporated in vacuo. The residue was purified by flash chromatography (DCM—10% methanol in DCM (v/v) gradient 100:0 rising to 40:60) to give the title compound (864 mg, 70%) as a white solid. Purity based on LC-MS 96%.

LRMS (m/z): 284/328/406 [M−99/M-55/M+23]¹⁺

LC-MS r.t. (min): 1.13²

### Intermediate 2:

### 4-(6-azidohexanoyl)piperazine-1-carbohydrazide 2,2,2-trifluoroacetate

tert-butyl 2-(4-(6-azidohexanoyl)piperazine-1-carbonyl)hydrazine-1-carboxylate (50.0 mg, 130 μmol) was dissolved in a mixture of dichloromethane (1.00 mL) and TFA (1.00 mL) and the reaction mixture was stirred at room temperature. After 1 hour the reaction mixture was evaporated in vacuo and co-evaporated with dichloromethane (3×5 mL) to give the crude title product as a white solid.

LRMS (m/z): 284/307 [M+1/M+23]¹⁺

### SO1861-SC-azide

To SO1861 (60 mg, 0.032 mmol) and 4-(6-azidohexanoyl)piperazine-1-carbohydrazide 2,2,2-trifluoroacetate (51.2 mg, 0.129 mmol) was added methanol (extra dry, 1.5 mL) and the reaction mixture was shaken for 1 min and left standing at room temperature. After 3 hours the reaction mixture was subjected to preparative MP-LC.^{1A} Fractions corresponding to the product were immediately pooled together, frozen and lyophilized overnight to give the title compound (55.6 mg, 81%) as a white solid.

Purity based on LC-MS 96%.

LRMS (m/z): 2127 [M−1]¹⁻

LC-MS r.t. (min): 3.39^{5A}

See FIG. 9 for the formula of SO1861-SC-azide (referred to as ‘SO1861-SC—N₃’ in FIG. 9).

Trivalent GalNAc (or GN3)-azide synthesis

### Intermediate 1:

### tert-butyl 1-azido-17,17-bis((3-(tert-butoxy)-3-oxopropoxy)methyl)-15-oxo-3,6,9,12,19-pentaoxa-16-azadocosan-22-oate

Intermediate 1 was produced as previously described in WO2022/055351 (page 136, line 3 to page 139, line 1, FIG. 8, Example 1C).

To di-tert-butyl 3,3′-((2-amino-2-((3-(tert-butoxy)-3-oxopropoxy)methyl)propane-1,3-diyl)bis(oxy))dipropionate (1.27 g, 2.51 mmol) was added a solution of 3-Azido(peg4)propionic acid N-hydroxysuccinimide ester (977 mg, 2.51 mmol) in DMF (10 mL). Next, DIPEA (657 μL, 3.77 mmol) was added and the reaction mixture was stirred overnight at room temperature. The reaction mixture was evaporated in vacuo and the residue was dissolved in ethyl acetate (100 mL). The resulting solution was washed with 0.5 N potassium bisulphate solution (2×100 mL) and brine (100 mL), dried over Na₂SO₄, filtered and evaporated in vacuo. The residue was purified by flash chromatography (DCM—10% methanol in DCM (v/v) gradient 100:0 rising to 0:100) to give the title compound (1.27 g, 65%) as a colorless oil. Purity based on LC-MS 100% (ELSD).

LRMS (m/z): 780 [M+1]¹⁺

LC-MS r.t. (min): 2.10²

### Intermediate 2:

### 1-azido-17,17-bis((2-carboxyethoxy)methyl)-15-oxo-3,6,9,12,19-pentaoxa-16-azadocosan-22-oic acid

Intermediate 2 was produced as previously described in WO2022/055351 (page 136, line 3 to page 139, line 1, FIG. 8, Example 1C).

To a solution of tert-butyl 1-azido-17,17-bis((3-(tert-butoxy)-3-oxopropoxy)methyl)-15-oxo-3,6,9,12,19-pentaoxa-16-azadocosan-22-oate (1.27 g, 1.63 mmol) in DCM (5.0 mL) was added TFA (5.0 mL, 65 mmol). The reaction mixture was stirred at room temperature. After 1.5 hours the reaction mixture was evaporated in vacuo, co-evaporated with toluene (3×10 mL) and DCM (3×10 mL) to give the crude title product as a colorless oil.

LRMS (m/z): 611 [M+1]¹⁺

### Intermediate 3:

### di-tert-butyl (10-(1-azido-3,6,9,12-tetraoxapentadecan-15-amido)-10-(13,13-dimethyl-5,11-dioxo-2,12-dioxa-6,10-diazatetradecyl)-5,15-dioxo-8,12-dioxa-4,16-diazanonadecane-1,19-diyl)dicarbamate

Intermediate 3 was produced as previously described in WO2022/055351 (page 136, line 3 to page 139, line 1, FIG. 8, Example 1C).

1-azido-17,17-bis((2-carboxyethoxy)methyl)-15-oxo-3,6,9,12,19-pentaoxa-16-azadocosan-22-oic acid (997 mg, 1.63 mmol), Oxyma Pure (1.04 g, 7.35 mmol) and EDCl·HCl (1.17 g, 6.12 mmol) were dissolved in DMF (10.0 mL). Next, DIPEA (1.99 mL, 11.4 mmol) was added, followed directly by the addition of a solution of N-BOC-1,3-propanediamine (1.07 g, 6.12 mmol) in DMF (10.0 mL). The reaction mixture was stirred overnight at room temperature. The reaction mixture was evaporated in vacuo and the residue was dissolved in ethyl acetate (100 mL). The resulting solution was washed with 0.5 N potassium bisulphate solution (100 mL), saturated sodium bicarbonate solution (2×100 mL) and brine (100 mL), dried over Na₂SO₄, filtered and evaporated in vacuo. The residue was purified by flash chromatography (DCM —10% methanol in DCM (v/v) gradient 0:100 rising to 100:0, staying at 100:0 until the product eluted) to give the title compound (1.16 g, 66%) as a yellowish viscous oil. LC-MS 99% (ELSD).

LRMS (m/z): 1080 [M+1]¹⁺

LC-MS r.t. (min): 1.513

### Intermediate 4:

### 3,3′-((2-((3-((3-aminopropyl)amino)-3-oxopropoxy)methyl)-2-(1-azido-3,6,9,12-tetraoxapentadecan-15-amido)propane-1,3-diyl)bis(oxy))bis(N-(3-aminopropyl)propanamide) tris(2,2,2-trifluoroacetate) synthesis

Intermediate 4 was produced as previously described in WO2022/055351 (page 136, line 3 to page 139, line 1, FIG. 8, Example 1C).

To a solution of di-tert-butyl (10-(1-azido-3,6,9,12-tetraoxapentadecan-15-amido)-10-(13,13-dimethyl-5,11-dioxo-2,12-dioxa-6,10-diazatetradecyl)-5,15-dioxo-8,12-dioxa-4,16-diazanonadecane-1,19-diyl)dicarbamate (1.16 g, 1.08 mmol) in DCM (10 mL) was added TFA (10 mL, 131 mmol). The reaction mixture was stirred at room temperature. After 2 hours the reaction mixture was evaporated in vacuo, co-evaporated with toluene (3×10 mL) and DCM (3×10 mL) to give the crude title product as a yellowish viscous oil.

LRMS (m/z): 260 [M+3]³⁺, 390 [M+2]²⁺, 780 [M+1]¹⁺,

### Intermediate 5:

### (2R,3R,4R,5R,6R)-5-acetamido-2-(acetoxymethyl)-6-((5-((2,5-dioxopyrrolidin-1-yl)oxy)-5-oxopentyl)oxy)tetrahydro-2H-pyran-3,4-diyl diacetate

Intermediate 5 was produced as previously described in WO2022/055351 (page 136, line 3 to page 139, line 1, FIG. 8, Example 1C). 5-(((2R,3R,4R,5R,6R)-3-acetamido-4,5-diacetoxy-6-(acetoxymethyl)tetrahydro-2H-pyran-2-yl)oxy)pentanoic acid (obtain according J. Am. Chem Soc., 2014, 136, 16958-16961, 3.00 g, 6.70 mmol) and N-Hydroxysuccinimide (926 mg, 8.05 mmol) were dissolved in DCM (50 mL). Next, EDCl·HCl (1.54 g, 8.05 mmol) and 4-(Dimethylamino)pyridine (82 mg, 0.67 mmol) were added and the reaction mixture was stirred overnight at room temperature. The reaction mixture was diluted with DCM and the resulting solution was washed with 0.5 N potassium bisulphate solution (150 mL), saturated sodium bicarbonate solution (150 mL) and brine (150 mL), dried over Na₂SO₄, filtered and evaporated in vacuo to give the title compound (3.60 g, 99%) as a white foam. Purity based on LC-MS 99% (ELSD).

LRMS (m/z): 545 [M+1]¹⁺

LC-MS r.t. (min): 1.073

### Intermediate 6:

### [(3R,6R)-3,4-bis(acetyloxy)-6-{4-[(3-{3-[2-(1-azido-3,6,9,12-tetraoxapentadecan-15-amido)-3-(2-{[3-(5-{[(2R,5R)-4,5-bis(acetyloxy)-6-[(acetyloxy)methyl]-3-acetamidooxan-2-yl]oxy}pentanamido)propyl]carbamoyl}ethoxy)-2-[(2-{[3-(5-{[(2R,5R)-4,5-bis(acetyloxy)-6-[(acetyloxy)methyl]-3-acetamidooxan-2-yl]oxy}pentana mido)propyl]carbamoyl}ethoxy)methyl]propoxy]propanamido}propyl)carbamoyl]butoxy}-5-acetamidooxan-2-yl]methyl acetate

Intermediate 6 was produced as previously described in WO2022/055351 (page 136, line 3 to page 139, line 1, FIG. 8, Example 1C).

3,3′-((2-((3-((3-aminopropyl)amino)-3-oxopropoxy)methyl)-2-(1-azido-3,6,9,12-tetraoxapentadecan-15-amido)propane-1,3-diyl)bis(oxy))bis(N-(3-aminopropyl)propanamide) tris(2,2,2-trifluoroacetate) (1.21 g, 1.08 mmol) was dissolved in a mixture of DMF (10 mL) and DIPEA (1.69 mL, 9.70 mmol). Next, (2R,3R,4R,5R,6R)-5-acetamido-2-(acetoxymethyl)-6-((5-((2,5-dioxopyrrolidin-1-yl)oxy)-5-oxopentyl)oxy)tetrahydro-2H-pyran-3,4-diyl diacetate (2.20 g, 4.04 mmol) was added and the reaction mixture was stirred over the weekend at room temperature. Next, the reaction mixture was evaporated in vacuo and the residue was purified by flash chromatography (DCM—30% methanol in DCM (v/v) gradient 0:100 rising to 100:0) to give the title compound (1.84 g, 83%) as a yellowish foam. LC-MS 95% (ELSD).

LRMS (m/z): 2068 [M+1]¹⁺

LC-MS r.t. (min): 1.183

### Intermediate 7:

### Trivalent GalNAc-azide

Trivalent GalNAc-azide was produced as previously described in WO2022/055351 (page 136, line 3 to page 139, line 1, FIG. 8, Example 1C).

[(3R,6R)-3,4-bis(acetyloxy)-6-{4-[(3-{3-[2-(1-azido-3,6,9,12-tetraoxapentadecan-15-amido)-3-(2-{[3-(5-{[(2R,5R)-4,5-bis(acetyloxy)-6-[(acetyloxy)methyl]-3-acetamidooxan-2-yl]oxy}pentanamido)propyl]carbamoyl}ethoxy)-2-[(2-{[3-(5-{[(2R,5R)-4,5-bis(acetyloxy)-6-[(acetyloxy)methyl]-3-acetamidooxan-2-yl]oxy}pentana mido)propyl]carbamoyl}ethoxy)methyl]propoxy]propanamido}propyl)carbamoyl]butoxy}-5-acetamidooxan-2-yl]methyl acetate (300 mg, 0.145 mmol) was dissolved in a mixture of triethylamine (2.00 mL, 14.4 mmol), methanol (2.00 mL) and water (2.00 mL) and the reaction mixture was stirred at room temperature. After 2 hours the reaction mixture was evaporated in vacuo. The residue was purified by preparative MP-LC.^{2B} Fractions corresponding to the product were immediately pooled together, frozen and lyophilized overnight to give the title compound (164 mg, 67%) as a white solid. Purity based on LC-MS 97%.

LRMS (m/z): 1688 [M−1]¹⁻

LC-MS r.t. (min): 1.99^{1A}

### Trivalent GalNAc-amine Formate

Trivalent GalNAc-amine formate was produced as previously described in WO2022/055351 (page 143-144, Example 1D).

Trivalent GalNAc-azide (36.5 mg, 21.6 μmol) was dissolved in a solution of potassium carbonate (5.97 mg, 43.2 μmol) in water (1.00 mL) and acetonitrile (1.00 mL). Next, a 1.0 M trimethylphosphine solution in THE (216 μL, 216 μmol) was added and the resulting mixture was shaken for 1 min and left standing at room temperature. After 45 min the reaction mixture was evaporated in vacuo and the residue was dissolved in water/acetonitrile (9:1, v/v, 1 mL). The resulting solution was directly subjected to preparative MP-LC.^{2B} Fractions corresponding to the product were immediately pooled together, frozen and lyophilized overnight to give the title compound (36.1 mg, 98%) as a white solid. Purity based on LC-MS 100%.

LRMS (m/z): 1662 [M−1]¹⁻

LC-MS r.t. (min): 1.62^{1A}

### Intermediate 14: Trivalent GalNAc (or GN3)-DBCO

Trivalent GalNAc-DBCO was produced as previously described in WO2022/055351 (page 143-144, Example 1D).

Trivalent GalNAc-amine formate (17.4 mg, 10.2 μmol) and DBCO-NHS (6.14 mg, 15.3 μmol) were dissolved in a solution of NMM (2.24 μL, 20.3 μmol) in DMF (0.50 mL). The reaction mixture was shaken for 1 min and left standing at room temperature. After 2 hours the reaction mixture was evaporated in vacuo and the residue was dissolved in water/acetonitrile (8:2, v/v, 1 mL). The resulting solution was directly subjected to preparative MP-LC.²c Fractions corresponding to the product were immediately pooled together, frozen and lyophilized overnight to give the title compound (14.2 mg, 72%) as a white solid. Purity based on LC-MS 96%.

LRMS (m/z): 1950 [M−1]¹

LC-MS r.t. (min): 1. 86^{1B}

### GN3-SC-SO1861

To SO1861-SC-N3 (18.0 mg, 8.45 μmol) and GN3-DBCO (16.5 mg, 8.45 μmol) was added a mixture of acetonitrile (250 μL) and 20 mM ammonium bicarbonate (750 μL). The reaction mixture was shaken for about 1 min and left standing at room temperature. After 1 hour the reaction mixture was subjected to preparative MP-LC.^{1A} Fractions corresponding to the product were immediately pooled together, frozen and lyophilized overnight to give the title compound (29.2 mg, 85%) as a white solid. Purity based on LC-MS 99%.

LRMS (m/z): 2038 [M−2H]²⁻

LC-MS r.t. (min): 2.19^{5B}

See FIG. 11 for the formula of GN3-SC-SO1861.

### GN3-AH-SO1861

To SO1861-AH-N3 (30.0 mg, 13.9 μmol) and GN3-DBCO (27.2 mg, 13.9 μmol) was added a mixture of acetonitrile (250 μL) and 20 mM ammonium bicarbonate (750 μL). The reaction mixture was shaken for about 1 min and left standing at room temperature. After 1 hour the reaction mixture was subjected to preparative MP-LC.^{1A} Fractions corresponding to the product were pooled together, frozen and lyophilized overnight to give the title compound (50.0 mg, 87%) as a white solid. Purity based on LC-MS 99%.

LRMS (m/z): 2049 [M−2H]2−

LC-MS r.t. (min): 2.155 B

See FIG. 10 for the formula of GN3-AH-SO1861.

### GN3-siTTR

Trivalent GalNAc-siRNA targeting murine transthyretin (also referred to as the nucleic acid component GN3-siTTR [SEQ ID No: 1 for the sense strand, and SEQ ID No. 15 for the antisense strand]) with advanced enhanced stability chemistry backbone was custom-produced by BioSpring Gesellschaft für Biotechnologie mbH, Germany, according to methods known in the art (FIG. 1). GalNAc monomers were conjugated via their phosphate groups in a linear fashion to generate a trimeric (trivalent) GalNAc. The phosphate group of the third GalNAc links to the 3′ end of the oligonucleotide sequence resulting in the following conjugate with sense strand [SEQ ID No: 1]: 5′-6*6*7685451315875756566000; and antisense strand [SEQ ID No: 15]: 5′-5*1*6564643668627275855*5*5; with 0=GalNac, 1=2′-Fluoro-U, 2=2′-Fluoro-A, 3=2′-Fluoro-C, 4=2′-Fluoro-G, 5=2′OMe-rU, 6=2′OMe-rA, 7=2′OMe-rC, 8=2′OMe-rG, *=Thioate. Such a linear trimeric (trivalent) GalNAc could also be conjugated to saponin components to generate for example GN3-SC-SO1861 or GN3-AH-SO1861 by methods known in the art.

### Cetuximab-AH-SO1861

To cetuximab (1087 mg, 4.800 mg/ml, 7.2×10-3 mmol, in TBS, 2.5 mM EDTA, pH 7.5) was added an aliquot of freshly prepared TCEP solution (1 mg/ml, 2.72 mole equivalents, 2.0×10-2 mmol, 5.65 mg), the mixture swirled by hand to mix then incubated for 210 minutes at 20° C. with roller-mixing. After incubation (prior to addition of SO1861-AH-Maleimide), a 2 mg (0.417 ml) aliquot of Ab-SH was removed and purified by gel filtration using zeba spin desalting column into TBS pH 7.5. This aliquot was characterized by UV-vis analysis and Ellman's assay (3.693 mg/ml, thiol to Ab ratio=4.0). To the bulk Ab-SH was added an aliquot of freshly prepared SO1861-AH-Maleimide solution (2 mg/ml, 5.2 mole equivalents, 3.8×10-2 mmol, 38.9 ml), the mixtures vortexed briefly then incubated for 120 minutes at 20° C. Besides the conjugation reaction, two aliquots of desalted Ab-SH (0.5 mg, 0.135 ml, 3.33×10-6 mmol) were reacted with NEM (8.00 equivalents, 2.66×10-5 mmol, 3.3 μg, 13.3 μl of a 0.25 mg/ml solution) or TBS pH 7.5 buffer (13.3 μl) for 120 minutes at 20° C., as positive and negative controls, respectively. After incubation (prior to addition of NEM), a ca. 2 mg (0.450 ml) aliquot of Ab—SO1861 mixture was removed and purified by gel filtration using zeba spin desalting column into TBS pH 7.5. This aliquot was characterized by UV-vis (3.271 mg/ml) and alongside positive and negative controls were characterized by Ellman's assay to obtain SO1861 incorporation. To the bulk Ab—SO1861 mixture was added an aliquot of freshly prepared NEM solution (2.5 mg/ml, 5 mole equivalents, 3.6×10-2 mmol, 4.54 mg) and the mixture stored at 2-8° C. overnight. The conjugate was purified by 10×40 cm Sephadex G50M column eluting with DPBS pH 7.5 to give purified cetuximab—SO1861 conjugate. The aliquot was filtered to 0.2 μm and dispensed. The result was a cetuximab—SO1861 conjugate. Yield=1056 mg, 97%, SO1861 to Ab ratio=3.9.

### Cetuximab-Cyanine5 (Cet-Cy5)

To cetuximab (5 mg, 3.30×10-5 mmol, 4.982 mg/ml, 1 mL in DPBS) was added an aliquot of freshly prepared Sulfo-Cy5-NHS solution (100 mg/ml, 320 mole equivalents) followed by an aliquot of DPBS pH 7.5 buffer (to normalize reaction volumes), the mixture vortexed briefly then incubated for 120 minutes at 20° C. with roller-mixing. After, to reaction mixture was added an aliquot of freshly prepared glycine solution (100 mg/ml, 5 mole equivalents with respect to Sulfo-Cy5) to quench the reaction. The conjugate was purified by gel filtration using PD10 G25 columns eluting into DPBS pH 7.5. The resulting conjugate was characterized by UV-vis spectrophotometry and BCA assay to ascertain antibody concentrations and Sulfo-Cy5 incorporations. TLC using methanol as mobile phase was conducted to show that residual free Sulfo-Cy5 was present. The conjugate was further purified by Dialysis using float-a-lyser G2 dialysis devices against DPBST pH 7.5 (dialysate changes were made after 12, 21, 84 and 108 h). The conjugate was then analyzed by BCA assay to ascertain antibody concentration and filtered to 0.2 μm under laminar flow (UV-vis spectrophotometry was used to ascertain losses due to filtration were negligible). Employing careful handling techniques and handling under laminar flow where possible, the conjugate was concentrated via diafiltration using vivaspin T4 centrifuge filter tubes (2,000 g, 20 minutes, 5° C.). Sample was removed for characterization and the bulk product characterized by UV-vis spectrophotometry and BCA assay to ascertain antibody concentrations and Sulfo-Cy5 incorporations. Yield: 29%, Sulfo-Cy5 incorporation: 51.3, Purity: 98.1%.

### GN3-siAT3

Trivalent GalNAc-siRNA targeting human SERPINC1 (and being cross-reactive with cercopithecine SERPINC1 of the non-human primate Macaca fascicularis), also referred to as the nucleic acid component GN3-siAT3 (SEQ ID No: 16 for the sense strand, and SEQ ID No. 17 for the antisense strand) with enhanced stability chemistry backbone was custom-made and manufactured by Biotage, United Kingdom (using the antisense-strand produced by BioSpring, Gesellschaft für Biotechnologie mbH, Germany, according to methods known in the art (FIG. 1)). GalNAc monomers were conjugated via their phosphate groups in a linear fashion to generate a trivalent GalNAc. The phosphate group of the third GalNAc links to the 3′ end of the oligonucleotide sequence resulting in the following conjugate with sense strand [SEQ ID No: 16]: 5′-6*G*8U5A7A775U8U5C8U7A5333; and antisense strand [SEQ ID No: 17]: 5′-U*8*G5A6U5A5UGG8G8U5A7C*A*G; with 3=GalNAc C3; 5=2′-Fluoro-A; 6=2′-Fluoro-G; 7=2′-Fluoro-C; 8=2′-Fluoro-U; Upper case letters=2′-O-methyl; *=phosphorothioate linkage.

### RNA Isolation and Gene Expression Analysis

A sample of ˜50-100 mg was cut from frozen liver tissue. Each sample was cut into smaller fragments, transferred into a 2.0 ml (RNase free) safe-lock tube, and 1 ml TRIzol™ Reagent (Fisher Scientific) and a 5 mm stainless steel bead (Qiagen) were added. The tube was placed in a TissueLyser II (Qiagen) for 5 minutes at 30 Hz, to completely disrupt the tissue. Then, 0.2 ml chloroform was added and the tube was shaken vigorously for 15 seconds. Samples were incubated for 2-3 minutes at room temperature, following centrifugation at 12.000×g for 15 minutes at 4° C. Next, 400 μl of the upper phase was taken and transferred to a new 1.5 ml collection tube. To this, 400 μl (RNase free) isopropanol was added and the solution was mixed gently and then placed overnight at 20° C. to allow the RNA to precipitate. Next, samples were centrifuged for 45-60 minutes at 15,000×g at 4° C. The supernatant was removed and the pellet was washed with 500 μl of 70% (RNase free) ethanol. Again, samples were centrifuged at 15,000×g for 5-10 minutes at 4° C. and the supernatant was removed. The RNA pellet was allowed to airdry before adding 500 μl nuclease free water. Samples were left at room temperature to dissolve the pellet. Subsequently, samples were mixed, placed on ice, and the RNA concentration was measured by Nanodrop One (Thermo Fischer).

Expression levels of SERPINC1 and reference genes (DDX3X and TBP) were measured using qRT-PCR with specific DNA primers, listed in Table A4. Each analysis reaction was performed in triplicate. Data analysis was done by the ΔCt method to determine SERPINC1 expression relative to the average expression of the two reference genes.

TABLE A4
Primers used in qPCR are shown below:

| Gene | Primer | Sequence (5′-3′) |
|---|---|---|
| SERPINC | Forward | CCAAGCTGGGTGCCTGTAA [SEQ ID NO: 18] |
| | Reverse | GAGTCGGCAGTTCAGTTTGG [SEQ ID NO: 19] |
| DDX3X | Forward | GTGGAAGTGGATCAAGGGGA [SEQ ID NO: 20] |
| | Reverse | CCAAAGCCACTTCTGTCACC [SEQ ID NO: 21] |
| TBP | Forward | AGAAGGTCTTGTGCTCACCC [SEQ ID NO: 22] |
| | Reverse | CGTCGTCTTCCTGAATCCCT [SEQ ID NO: 23] |

### Example 1: Depot Release by Saponin Components In Vivo: Tolerability, Efficacy and Durability of Effect of GN3-siRNA Under Influence of (Co-)Administration of Saponin Components

### In Vivo Study Design

In total, 69 male C57BL/6 mice allocated to twelve dosing groups (vehicle n=3, GN3-siTTR n=6, and variously timed combinations of GN3-siTTR (also referred to as trimeric GalNAc-siRNA or trivalent GalNAc-siRNA targeting murine Ttr, all n=6) and saponin components (here, GN3-SO1861 with either AH- or SC-linker for SO1861, also referred to as trimeric GalNAc-AH-SO1861, trivalent GalNAc-AH-SO1861 or GN3-AH-SO1861 and trimeric GalNAc—SC-SO1861, trivalent GalNAc—SC-SO1861 or GN3-SC-SO1861, respectively), were dosed with the test compounds (GN3-siTTR always at 0.3 mg/kg and GN3-SO1861 always at 1 mg/kg), see Table A1 for the dosing groups. Blood sampling was generally performed on day −4, 3, 7, 10, 14, 17, 21, 24, 28, 31, 35 and 49, unless indicated otherwise in Table A1; for ethical reasons, per group, blood draws were divided over two cohorts of mice as indicated by mouse ID. After blood sampling, serum was prepared and aliquoted in 2 tubes. One aliquot was used for serum TTR protein analysis and the other one for serum ALT enzyme analysis. Mice were terminated on day 49.

### Bioanalysis TTR Protein

To assess efficacy of the treatment, serum samples were analyzed for TTR protein content by ELISA using the ALPCO Mouse Prealbumin ELISA Kit (#41-PALMS-Ed1, ALPCO) according to the manufacturer's instructions.

### Clinical Chemistry

To assess tolerability, ALT protein as a reporter for liver tolerability was assessed in serum samples with a Roche COBAS 6000 analyzer.

TABLE A1
Dosing groups in vivo tolerability, efficacy and durability of different
dosing regimens of trimeric GalNAc-SO1861 (GN3-AH-SO1861 and GN3-SC-
SO1861) in combination with trimeric GalNAc-siRNA (GN3-siTTR)

| Treatment, | Treatment, | Treatment | | |
|---|---|---|---|---|
| Component 1 | Component 2 | day | Mouse | Blood sampling |
| 0.3 mg/kg | 1 mg/kg | Component 2 | ID | Timepoint |
| GN3-siTTR | N/A | N/A | 1-3 | −4 d, 3 d, d 14, d 28, d 49 |
| | | | 4-6 | −4 d, 7 d, d 21, d 35, d 49 |
| GN3-siTTR | GN3-AH-SO1861 | 0 (Co-Admin) | 7-9 | −4 d, 3 d, d 14, d 28, d 49 |
| | | | 10-12 | −4 d, 7 d, d 21, d 35, d 49 |
| GN3-siTTR | GN3-AH-SO1861 | 7 | 13-15 | −4 d, 3 d, d 10, d 21, d 35, d 49 |
| | | | 16-18 | −4 d, 7 d, d 14, d 28, d 49 |
| GN3-siTTR | GN3-AH-SO1861 | 14 | 19-21 | −4 d, 3 d, d 14, d 21, d 35, d 49 |
| | | | 22-24 | −4 d, 7 d, d 17, d 28, d 49 |
| GN3-siTTR | GN3-AH-SO1861 | 21 | 25-27 | −4 d, 3 d, d 14, d 24, d 35, d 49 |
| | | | 28-30 | −4 d, 7 d, d 21, d 28, d 49 |
| GN3-siTTR | GN3-AH-SO1861 | 28 | 31-33 | −4 d, 3 d, d 14, d 28, d 35, d 49 |
| | | | 34-36 | −4 d, 7 d, d 21, d 31, d 49 |
| GN3-siTTR | GN3-SC-SO1861 | 0 (Co-Admin) | 37-39 | −4 d, 3 d, d 14, d 28, d 49 |
| | | | 40-42 | −4 d, 7 d, d 21, d 35, d 49 |
| GN3-siTTR | GN3-SC-SO1861 | 7 | 43-45 | −4 d, 3 d, d 10, d 21, d 35, d 49 |
| | | | 46-48 | −4 d, 7 d, d 14, d 28, d 49 |
| GN3-siTTR | GN3-SC-SO1861 | 14 | 49-51 | −4 d, 3 d, d 14, d 21, d 35, d 49 |
| | | | 52-54 | −4 d, 7 d, d 17, d 28, d 49 |
| GN3-siTTR | GN3-SC-SO1861 | 21 | 55-57 | −4 d, 3 d, d 14, d 24, d 35, d 49 |
| | | | 58-60 | −4 d, 7 d, d 21, d 28, d 49 |
| GN3-siTTR | GN3-SC-SO1861 | 28 | 61-63 | −4 d, 3 d, d 14, d 28, d 35, d 49 |
| | | | 64-66 | −4 d, 7 d, d 21, d 31, d 49 |
| Vehicle (PBS) | — | — | 67-69 | −d 4, 14 d, 28 d, 49 d |

### Example 2: Depot Release of Liver Targeted siRNAs by Saponin Components In Vitro: Efficacy and Durability Enhancement of GN3-siRNA Under Influence of Saponin Components

### Mouse Primary Hepatocyte Treatment

Wells of a 24- or 96-well plate (Greiner BioOne) were coated overnight at 37° C. with rat collagen-I (Ibidi). Next day the coating solution was removed and the wells were washed 1× with PBS (PAN-Biotech GmbH). Hepatocyte Plating Medium (HPM, PRIMACYT Cell Culture Technology GmbH) was added using 210 μl or 35 μl per well, per 24-wp or 96-wp respectively. Next, GN3-siTTR/DPBS was dissolved at 10× the final concentration (final concentration is 0.5 nM or 0.05 nM GN3-siTTR, as indicated) and 90 μl or 15 μl was added to each well, per 24-wp or 96-wp respectively. Cryopreserved primary mouse hepatocytes (Cytes Biotechnology) were thawed in Hepatocyte Thawing Medium (HTM, PRIMACYT Cell Culture Technology GmbH) and collected by centrifugation at 50×g for 10 min. Cells were gently re-suspended in HPM at a density of 0.215×10⁶ cells/ml. Cells were added on the top of the medium and treatment mix at 150,000 cells/well or 35,000 cells/well, respectively for the 24- or 96-well plates. After 6 hrs of seeding/treatment, medium was removed and cells were washed with HPM. Subsequently 810 μl (24-well) or 135 μl (96-well) Hepatocyte Maintenance Medium (HMM, PRIMACYT Cell Culture Technology GmbH) was added per well, after which saponin components were added from a 10× concentrated stock solution in PBS or PBS was added for control samples followed by a 24 hr or 48 hr incubation, as indicated in FIG. 3A. Alternatively, cells were incubated in the HMM for 18 hrs, after which either saponin components or PBS were added for an additional 3 or 6 hrs, as indicated in the experiment, followed by wash and then a further incubation in HMM for 21 or 18 hrs, as indicated in FIG. 3C. Plates were incubated with saponin components, in concentration of 800 nM SO1861; 2000 nM SO1861-AH-Maleimde-Block (also referred to as SO1861-AH-Block), or 250 nM GN3-AH-SO1861. At the end of the experiment, cells were harvested for gene expression and cell viability analysis.

### Cell Viability Assay (CTG)

After treatment, the cell viability was determined by a CTG-assay, performed according to the manufacturer's instruction (CellTiter-Glo@2.0 Cell Viability Assay, Promega). Briefly, the cell plate was first equilibrated to RT for 30 minutes. Next, to each well containing 150 μL treatment medium 100 μL CTG solution was added. The plate briefly mixed (10 sec, 600 rpm) and incubated for 10 minutes in the dark at RT. Subsequently, the luminescence signal was measured on a Spectramax ID5 μlate reader (Molecular Devices). For quantification, the background signal of ‘medium only’ wells was subtracted from all other wells before the cell viability percentage of treated/untreated cells was calculated by dividing the background corrected signal of treated wells over the background corrected signal of the untreated wells (×100).

RNA Isolation and Gene Expression Analysis from Mouse Primary Hepatocytes

RNA from cells was isolated using TRIzol™ Reagent (Thermo Scientific) according to the manufacturer's instruction. Conversion into cDNA was performed using iScript™ cDNA Synthesis Kit (BioRad) using standard protocols. Ttr expression levels and levels of specific hepatocyte housekeeping genes were determined using quantitative real-time PCR assays (qRT-PCR) using iTaq™ Universal SYBR® Green Supermix (BioRad) and the Light Cycler 480 II (Roche Diagnostics) with specific DNA primers, listed in Table A2. Analysis was done by the ΔCt method to determine Ttr expression relative to 2 hepatocyte-specific housekeeping control mRNAs. Each analysis reaction was performed in triplicate.

TABLE A2
Primers used in qRT-PCR analysis of mouse primary hepatocytes

| | | |
|---|---|---|
| Ttr | Forward | TTTCACAGCCAACGACTCTG [SEQ ID NO: 2] |
| | Reverse | AGAATGCTTCACGGCATCTT [SEQ ID NO: 3] |
| Mm Ppia | Forward | GCGGCAGGTCCATCTACG [SEQ ID NO: 4] |
| | Reverse | GCCATCCAGCCATTCAGTC [SEQ ID NO: 5] |
| Mm Sdha | Forward | GAGGAAGCACACCCTCTCAT [SEQ ID NO: 6] |
| | Reverse | GGAGCGGATAGCAGGAGGTA [SEQ ID NO: 7] |

### Results

Efficacy and In Vivo Durability of 0.3 mg/kg Trivalent GalNAc-siTTR is Markedly Improved by Administration of 1 mg/kg Saponin Component, Especially when Added Delayed and then Independently of Dosing Schedule

Trivalent GalNAc is a targeting ligand that recognizes and binds the ASGPR1 receptor on hepatocytes. For the two saponin components used in this example, trivalent GalNAc was produced as previously described. SO1861-AH-N₃ was conjugated (in a similar manner as described in FIG. 10 to trivalent GalNAc, with a DAR=1 for SO1861 to yield trivalent GalNAc-AH-SO1861, also referred to as (GalNAc)3-AH-SO1861, or GN3-AH-SO1861). Likewise, SO1861-SC—N₃ was conjugated (in a similar manner as described in FIG. 11, to trivalent GalNAc, with a DAR=1 for the bound SO1861, to yield trivalent GalNAc-SC-SO1861, also referred to as (GalNAc)3-SC-SO1861, or GN3-SC-SO1861. Trivalent GalNAc-siRNA targeting the murine Ttr (also referred to as GN3-siTTR [SEQ ID NO: 1]) was custom-produced (FIG. 1).

All mice were intravenously (IV) injected with 0.3 mg/kg GN3-siTTR on day 0, except 3 control mice that were injected with vehicle (PBS) only on day 0. Then, all GN3-siTTR-dosed mice, except one benchmark group of 6 animals, additionally received saponin components at a dose of 1 mg/kg, either on day 0, or day 7, or day 14, or day 21, or day 28. Serum samples were taken at different timepoints before and after dosing (Table A1) to assess the effect of the GN3-siTTR on circulating TTR protein levels. As FIG. 2A, 2B, 2C, 2D, 2E shows, as expected, mice receiving vehicle (n=3, on day 0) showed constant and high levels of TTR protein in serum throughout the study (i.e. at −4, 14, 28, and 49 days). As expected, mice receiving only GN3-siTTR (n=6, on day 0, 0.3 mg/kg) without a saponin component showed a maximum effect of ˜80% TTR knockdown approximately 7-14 days after treatment with said GN3-siTTR, and with a return of TTR protein levels to baseline levels (i.e., the level measured before administration, and also the level comparable to vehicle treated mice) on day 49. In contrast, and in stark contrast to vehicle and to GN3-siTTR-only treated mice, mice receiving a combination of GN3-siTTR (on day 0, 0.3 mg/kg) and the saponin component GN3-SC-SO1861 (on day 0, 1 mg/kg) achieved far greater reduction (of >95%) already on day 3, the earliest day of serum TTR protein assessment (FIG. 2A). Surprisingly, mice that had received GN3-siTTR on day 0 and then a dose of saponin component either 7 days later (FIG. 2B), or 14 days later (FIG. 2C, or 21 days later (FIG. 2D), or 28 days later (FIG. 2D) showed almost complete TTR protein knockdown (>95%) within 3-7 days after treatment with the saponin component. Also irrespectively of when the saponin component was administered (in relation to the administration GN3-siTTR administration timepoint), there was no loss of durability of effect: saponin compound administered at either day 7, or day 14, or day 21, or day 28 after initial GN3-siTTR administration, resulted in up to 80% TTR serum level reduction for any of these regimens at the end of study at day 49 (FIG. 2B, 2C, 2D, 2E). Any of these co-treatment regimens were also equally well tolerated, as in general, no saponin-compound-induced increase in the ALT enzyme levels which absence of an increased level serves as a proxy of liver tolerability was observed (not shown). Taken together, this data shows that co-administration of a saponin component either at the same time, but more so at a delayed time point, to GN3-siTTR significantly increases efficacy, measured in this example as protein reduction, compared to treatment with the same dose of GN3-siTTR alone. Marked to almost complete reduction (>95%) of TTR protein expression by 0.3 mg/kg GN3-siTTR was reached only following co-dosing with a saponin compound GN3-SC-SO1861, but independently of the timepoint of saponin compound administration, i.e., either at day 7, or day 14, or day 21, or day 28 throughout the study. Results obtained with saponin component GN3-AH-SO1861 were similar to largely identical (not shown). Taken together these results confirm that saponin components can mediate efficient release of the siRNA from the endosomal (depot) compartment over at least 28 days after initial dosing of the siRNA compound. Additionally, no loss of durability of effect was observed. Remarkably, in all cases where treatment was sequential (i.e., GN3-siTTR on day 0 and saponin compound addition after 7, 14, 21, or 28 days), the levels of TTR were reduced by 80% at day 49 after initial GN3-siTTR treatment. Taken together, saponin components were not only able to enhance the efficacy of a 0.3 mg/kg dose of GN3-siTTR, but a timed, saponin-induced release markedly prolonged the durability of effect.

### Example 3: Depot Release of Liver Targeted siRNAs by Saponin Component In Vitro: Efficacy and Durability Enhancement of GN3-siRNA Under Influence of Saponin Components

The timed, saponin-component-inducible release of a nucleic acid from endosomes after prior loading cells with the nucleic acid was also assessed in vitro in murine primary hepatocytes (MPH) in different dosing regimens. To this end, MPH were exposed to 0.05 nM GN3-siTTR for 6 hrs to allow uptake. The cells were then washed with medium and incubated with medium containing saponin components or medium with equivalent amounts of PBS as control for an additional 24 hrs or 48 hrs, after which remaining Ttr RNA levels were analysed by qRT-PCR. MPH receiving no saponin components served as control (see FIG. 3A for the incubation scheme). Compared to control cells, a loading of 6 hrs in 0.05 nM GN3-siTTR resulted in 62% remaining Ttr levels at 24 hrs after the removal of the GN3-siTTR (FIG. 3B). In contrast, when MPH were exposed to saponin components for 24 hrs after the initial 6 hrs loading with GN3-siTTR, only approximately 5-15% Ttr levels remained, for all saponin components (SO1861, SO1861-AH-Maleimide-Block (saponin according to formula (V), also referred to as SO1861-AH-Block), or GN3-AH-SO1861) (FIG. 3B). These results were comparable when the incubation with saponin components or PBS was prolonged to 48 hrs, following GN3-siTTR (see FIG. 3A for the incubation scheme). The remaining Ttr RNA level slightly decreased for 0.05 nM GN3-siTTR followed by 48 hrs of PBS to 54% at the experiment end (FIG. 3C), compared to the 62% after 24 hrs (FIG. 3B). For timed release in cells treated for 48 hrs by saponin components, the Ttr levels were reduced down to 3-4% for saponin components with acylhydrazone (AH), i.e. SO1861-AH-Maleimide-Block and GN3-AH-SO1861, respectively (FIG. 3C), and to 10% for the saponin component SO1861 (FIG. 3C). Taken together, these data show that, surprisingly, saponin components can increase potency of a preloaded GN3-siTTR in a timed and inducible manner after a short 6 hr-loading of GN3-siTTR.

The timed, saponin-component-inducible release was also assessed after a short pulse of saponin component (FIG. 3D for experiment set up and FIG. 3D for results) in murine primary hepatocytes (MPH). To this end, MPH were exposed to 0.5 nM GN3-siTTR for 6 hrs to allow uptake. The cells were then washed and incubated with medium for 18 hrs (resting period) before adding saponin components (or medium with PBS for control) for only 6 hrs. After this release pulse, cells were washed and placed in full medium again until analysis. The remaining Ttr RNA levels were analysed by qRT-PCR. MPH receiving no GN3-siRNA and no saponin components served as normalization control (100% Ttr RNA, FIG. 3E). MPH cells loaded with GN3-siTTR for 6 hrs followed by only medium/PBS treatments, but no saponin component pulse served as GN3-siTTR efficacy controls; this treatment resulted in 10% remaining Ttr levels (FIG. 3E). In contrast, when MPH were exposed to saponin components for only 6 hrs after the initial GN3-siTTR loading+resting period in medium, only approximately 4-7% Ttr levels remained, irrespective of which saponin component (SO1861, SO1861-AH-Maleimide-Block (also referred to as also referred to as SO1861-AH-Block), or GN3-AH-SO1861) was added. Thus, the saponin component can trigger the GN3-siTTR delivery on target also with a delay after a resting period. To assess the exposure time required to elicit release, the pulse of saponin component was reduced even further to 3 hrs after the resting period, followed medium incubation until the end of the experiment. Here, the reduction was in all cases comparable to the longer pulse of 6 hrs with saponin component, i.e. around 11% for 0.5 nM GN3-siTTR alone, but 3-7% for the saponin-component-pulsed conditions, irrespective of which saponin component was used (data not shown). Thus, the pulse with saponin component could be halved without affecting the achieved efficacy. Taken together, these data show that saponin components can increase potency of a preloaded GN3-siRNA in a timed and inducible manner after a rest period and a short “release pulse” of saponin-component, underscoring a preload dosing regimen followed by short exposure to a saponin components.

### Example 4: Depot Release in Epidermoid Carcinoma and Metastatic Melanoma Cells by Saponin Components In Vitro: Efficacy and Durability Enhancement of STAT3 ASO Under Influence of Saponin Components

A431 and A2058 cell Treatment

Cells were cultured in DMEM (PAN-Biotech GmbH) supplemented with 10% fetal bovine serum (FBS) (PAN-Biotech GmbH) and Pen/Strep (PAN-Biotech GmbH) at 37° C. and 5% CO₂. Cells were seeded in a 24-well plate at 30.000 cells/well in 600 μL/well and incubated overnight at 37° C. The next day, 10× concentrated compound-mix samples were prepared in PBS, which contained the compounds as indicated in the experimental set up (FIG. 4A, 4B, 4C, 4D), i.e. PBS, an antisense oligonucleotide (ASO) and/or saponin component at 10× final concentration. Additional 210 μL culture medium was added to the wells, followed by the addition of 90 μL compound-mix/well. Cells were treated with either ASO or PBS vehicle control for 6 or 24 hrs, followed by wash and either immediate saponin component in medium followed by treatment for 48 hrs or 6 hrs/24 hrs resting period (medium incubation), followed by subsequent addition of saponin component treatment for 48 hrs as indicated in the experimental set up (FIG. 4A, 4B, 4C, 4D). Alternatively, cells were treated with ASO and saponin component mix for 48 hrs without medium change, all at 37° C. Times and details of treatment are as indicated in the experimental set up (FIG. 4A, 4B, 4C, 4D). At the end of the experiment, cells were harvested for gene expression analyses.

### STAT3 ASO

A STAT3 antisense oligonucleotide (ASO) with the following sequence and following modification [SEQ ID NO: 8]: 3*1*2*T*T*T*G*G*A*T*G*T*0*2*4*3, with 0=5-Methyl-dC, 1=2′MOE-5Me-rU, 2=2′MOE-rA, 3=2′MOE-5Me-rC, 4=2′MOE-rG, *=Thioate, was produced by BioSpring Gesellschaft für Biotechnologie GmbH, Germany, according to methods known in the art.

RNA Isolation and Gene Expression Analysis from A431 and A2058 Cells RNA from cells was isolated using TRIzoI™ Reagent (Thermo Scientific) according to the manufacturer's instruction. Conversion into cDNA was performed using iScript™ cDNA Synthesis Kit (BioRad) using standard protocols. STAT3 expression levels and levels of housekeeping genes were determined using quantitative real-time PCR assays (qRT-PCR) using iTaq™ Universal SYBR@Green Supermix (BioRad) and the Light Cycler 480 II (Roche Diagnostics) with specific DNA primers, listed in Table A3. Analysis was done by the ΔCt method to determine STAT3 expression relative to 2 housekeeping control mRNAs, HBMS and SDHA. Each analysis reaction was performed in triplicate.

TABLE A3
Primers used in qRT-PCR analysis of A431 and A2058 cells

| | | |
|---|---|---|
| STAT3 | Forward | ACATGCCACTTTGGTGTTTCATAA [SEQ ID NO: 9] |
| | Reverse | TCTTCGTAGATTGTGCTGATAGAGAAC [SEQ ID NO: 10] |
| HBMS | Forward | CACCCACACACAGCCTACTT [SEQ ID NO: 11] |
| | Reverse | GTACCCACGCGAATCACTCT [SEQ ID NO: 12] |
| SDHA | Forward | GCATTTGGCCTTTCTGAGGC [SEQ ID NO: 13] |
| | Reverse | CTCCATGTTCCCCAGAGCAG [SEQ ID NO: 14] |

### Example 5: Depot Release in Epidermoid Carcinoma Cells and Metastatic Melanoma Cells by Saponin Components In Vitro

To assess depot release by saponin components extrahepatically, human A431 epidermoid carcinoma cells and A2058 metastatic melanoma cells were incubated with a STAT3-targeting ASO and various saponin components (SO1861, SO1861-AH-Maleimide-Block, or Cet-AH-SO1861) at different time points, as illustrated in FIG. 4A, 4B, 4C, 4D.

Treatment for 48 hrs with ASO alone caused a significant reduction in STAT3 expression in A431 cells to a residual 32%, but co-administration of ASO plus a saponin component (both targeted and non-targeted) showed an increased reduction down to 11% to 18% (FIG. 4E), irrespective of which saponin component was used. In A2058 cells a similar effect was observed, to the end that ASO alone caused a reduction in STAT3 expression to 75% but the combination treatment of ASO+(non-targeted) saponin component showed an increased reduction, even down to down to 4% to 5% (FIG. 4I). Expectedly, the targeted saponin component Cet-AH-SO1861 did not enhance STAT3 silencing in A2058 cells, as these cells do not express the cetuximab-target, i.e. the EGF receptor (data not shown). Taken together, the data shows that at the concentrations and experiment duration examined, only co-treatment of the ASO and the saponin component is highly effective in reducing STAT3 expression in multiple human carcinoma cell types to 11%-18% in A431 and 4% to 5% in A2058, respectively.

Next, we tested whether these extrahepatic carcinoma cells contain an endosomal effector depot and whether also non-targeted nucleic acids could be loaded and effectively released with saponin components in a time resolved manner in these cells. To this end, the STAT3 ASO was first loaded into cells. Cells were then washed after 24 hrs, followed either directly by a saponin component incubation or following a 6 hrs or a 24 hrs resting period in medium (as described in FIG. 4B, 4C, 4D) before saponin components were added to the cells. In benchmark conditions, when the STAT3 ASO loading was followed by control (PBS) incubation, STAT3 expression was slightly reduced to 87% in A431 cells (FIG. 4F), and an additional resting period of 6 hrs did not further reduce the STAT3 mRNA levels (also 87%, FIG. 4G), while an additional 24 hrs in PBS lead to a remaining 43% STAT3 levels (FIG. 4H). In contrast, treatment with saponin components directly after ASO loading caused a significant decrease in STAT3 RNA levels, reducing it to 12% or 42%, following application of non-targeted or targeted saponin components, respectively (FIG. 4F). When an additional resting period of 6 hrs was included, all saponin components reduced the STAT3 expression very efficiently to only 20% in A431 cells (FIG. 4G) However, a resting period of 24 hrs even reduced it further to 10%-13% (FIG. 4H). In A2058 cells similar effects were observed: when the STAT3 ASO loading was followed by control treatment (PBS), it did not reduce the STAT3 expression in this set up (99% remaining, FIG. 4J). Neither did a resting period of 6 hrs (FIG. 4K) nor 24 hrs (FIG. 4L) change this result (97% and 83% STAT3 RNA remaining, respectively). In stark contrast, the addition of a (non-targeted) saponin component effectively reduced the STAT3 ASO levels to 4-16%, irrespective of the (non-targeted) saponin component used and whether it was added directly after the ASO loading FIG. 4J), or following a 6 hr resting period (FIG. 4K), or following a 24 hr-resting period FIG. 4L). Again, as expected, the Cet-AH-SO1861 was not effective on A2058 cells, as these cells lack the EGFR (data not shown). In all cases, no marked effect on cell viability, as monitored by CTG assay, was observed (data not shown).

Combined, this data shows that the endosomal effector depot of carcinoma cells loaded with a nucleic acid can be reached by saponin components resulting in superior efficacy. When addition of the saponin component to the cells is delayed with respect to addition of payload (here the nucleic acid component), the longer resting periods achieved efficacies that were at least similar but in most cases were even superior to co-incubations of the nucleic acid component and the saponin component. Both cell lines used clearly show evidence to suggest that a STAT3 ASO depot is formed that can be targeted by saponin components up to at least 24 hr after the removal of the ASO. Even after this 24 hr resting period, saponin components can still induce maximum potency of the STAT3 ASO, which is shown by the high efficacy in the sequential mode (with washout or resting periods) compared to 48 hr direct co-administration of the nucleic acid component and saponin component (FIG. 4A-4D experimental set up, FIG. 4E-4L results).

### Example 6: Visualized Gradual Depot Release in Enidermoid Carcinoma Cells by Saponin Compounds In Vitro

### A431 Cell Treatment and Image Recording

Cells were cultured in DMEM (PAN-Biotech GmbH) supplemented with 10% fetal bovine serum (FBS) (PAN-Biotech GmbH) and Pen/Strep (PAN-Biotech GmbH) at 37° C. and 5% CO2. Cells were seeded in a 96-well plate at 5.000 cells/well in 100 μL/well and incubated overnight at 37° C. The next day, Cy5-labeled cetuximab (Cet-Cy5) was added to a final concentration of 2 nM in a total volume of 200 μL/well and the cells were incubated at 37° C. (FIG. 5A). After 48 hrs medium was removed and the cells were washed with PBS and then fresh culture medium was added. The cell plate was placed in the incubator of a temperature-controlled imaging system (xCELLigence RTCA eSight (Agilent)). Imaging was started and every hour an image of the brightfield and Cy5 fluorescence was recorded. Then, 72 hrs after the start of Cet-Cy5 treatment, saponin components or PBS as control was added, and imaging continued until the end of the experiment at 160 hrs.

eSight Image Analysis

Brightfield and Cy5 fluorescence images were collected every hour between 48 hrs and 160 hrs of the experiment. From these images, the fluorescence intensity in the Cy5 channel was analyzed and the total integrated intensity (RI×μm2 per image) was calculated and plotted as function of time of experiment.

### Visualized Gradual Depot Release by Different Saponin Components

A431 cells were incubated according to the scheme in FIG. 5A. To this end, cells were first loaded with a fluorescently Cy5-labeled cetuximab (Cet-Cy5) for 48 hrs, followed by a wash and incubation with culture medium. As FIG. 5B shows the intensity of the Cy5-signal continues to build up in this resting phase, being interpreted as the (continued) uptake of Cet-Cy5 from the medium and internalisation of Cet-Cy5 that is EGFR bound, which is highly expressed on A431 cells. In the medium, the Cy5 signal is only weakly detectable as it is diffuse and diluted, while after uptake it becomes accumulated in the cells and endosomes, where it is concentrated and thus fluorescence intensity can be clearly visualized. The total intensity of fluorescence increases over time per RI×μm2 per image. After this resting period, saponin components (SO1861 or Cet-AH-SO1861), or PBS as control, were added. The result is an onset of gradual fluorescence intensity loss under influence of different saponin components, starting between 74-110 hrs. While the loss of Cy5-fluorescence intensity is gradual, it is very pronounced and the saponin component-treated A431 cells reach ‘zero fluorescent state’ at the end of the experiment at 160 hrs. In contrast, the loss of fluorescence intensity under influence of PBS is not only markedly slower but also does not reach ‘zero fluorescent state’ in the course of the experiment FIG. 5B. This data shows that under influence of saponin components, Cy5 is gradually and improvingly released from endosomes, compared to PBS control. Notably, there is no instant, burst-like loss observed under the influence of the saponin components. Without wishing to be bound by any theory, this is indicative for a small-molecule release enhancing effect that leaves the endosomes from which the small molecule (here Cy5) releases, intact. This example illustrates that also (antibody-conjugated) small molecules release from endosomes can benefit from a delayed saponin administration.

### Example 7: Depot Release of Proteinaceous Payload (Toxin) in Epidermoid Carcinoma and Metastatic Melanoma Cells by Saponin Components

Anti-CD71-saporin Synthesis

Custom anti-CD71-saporin (aCD71-SPRN) conjugate, consisting of an antibody targeting CD71 and the protein toxin, saporin, was produced and purchased from Advanced Targeting Systems (San Diego, CA). CD71 antibody (anti-CD71, clone OKT-9, InVivoMab) was purchased from BioXCell.

### A431 and A2058 Cell Treatment

Cells were cultured in DMEM (PAN-Biotech GmbH) supplemented with 10% fetal bovine serum (FBS) (PAN-Biotech GmbH) and Pen/Strep (PAN-Biotech GmbH) at 37° C. and 5% CO2. Cells were seeded in a 96-well plate at 5.000-6.000 cells/well in 100 μL/well and incubated overnight at 37° C. The next day, 10× concentrated compound-mix samples were prepared in PBS, which contained the compounds as indicated in FIG. 6A, 6B, 6C, 6D, i.e. anti-CD71-saporin (aCD71-SPRN, protein toxin) and/or saponin components. The medium was removed from the cell culture plate and replaced by 180 μL fresh culture medium, followed by the addition of 20 μL 10× compound-mix/well. PBS treatment served as vehicle control. After 24 hrs treatment, the cells were washed and incubated with medium or medium containing PBS/saponin components for the time indicated in FIG. 6B, 6C, 6D at 37° C. At the end of the experiment at 48 hr after saponin component treatment or PBS treatment the cells were harvested for cell viability analysis. Alternatively, as a positive control, the cells were treated with a co-administration of protein toxin and saponin component (or PBS) mix for 48 hrs without medium change, FIG. 6A.

### Cell Viability Assay (MTS)

After treatment cell viability was determined by an MTS-assay, performed according to the manufacturer's instruction (CellTiter 96@AQueous One Solution Cell Proliferation Assay, Promega). Briefly, the MTS solution was diluted 20′ in DMEM without phenol red (PAN-Biotech GmbH) supplemented with 10% FBS. The cells were washed once with 200 μL/PBS well, after which 100 μL diluted MTS solution was added/well. The plate was incubated for approximately 20-30 minutes at 37° C. Subsequently, the OD at 492 nm was measured on a Spectramax iD5 μlate reader (Molecular Devices). For quantification the background signal of ‘medium only’ wells was subtracted from all other wells, before the cell viability percentage of treated/untreated cells was calculated, by dividing the background corrected signal of treated wells over the background corrected signal of the untreated wells (×100).

### Depot Release of Proteinaceous Payload (Toxin) in Epidermoid Carcinoma and Metastatic Melanoma Cells by Saponin Components In Vitro.

Immunotoxins consisting of a monoclonal antibody linked to the ribosome inactivating protein (RIP) saporin have been developed and evaluated in clinical trials in patients with leukaemia and lymphoma.

One disadvantage of these types of immunotoxins for clinical use is their relatively narrow therapeutic window and associated potentially life-threatening toxicities at dose levels that are therapeutic. Here, the in vitro efficacy and enhancement of effect durability of an immunotoxin (anti-CD71-saporin, also referred to as aCD71-SPRN, protein toxin) by endosome escape enhancer saponin components was assessed in epidermoid carcinoma cells and metastatic melanoma cells. A concentration range of a saponin component was added either together with a fixed concentration of 5 pM aCD71-SPRN to the cancer cells, or at various time points after the loading of the cancer cells with a fixed concentration of 5 pM aCD71-SPRN (according to dosing schemes in FIG. 6A, 6B, 6C, 6D). As a negative control, cells were loaded with protein toxin according to scheme FIG. 6A-D but received medium with PBS instead of saponin component.

Dosing of the epidermoid carcinoma A431 cells with a fixed concentration of aCD71-SPRN and a concentration range of saponin component 1 (SO1861, FIG. 6E), saponin component 2 (SO1861-AH-Maleimide-Block (saponin molecule according to formula (V), also referred to as SO1861-AH-Block), FIG. 6F) or saponin component 3 (cetuximab-AH-SO1861, FIG. 6G) shows that 48 hr co-administration of the toxin and saponin component together reduces cell viability and is efficacious at low doses of the immunotoxin (5 pM). Surprisingly, when the saponin component is not added together with aCD71-SPRN, but sequentially, i.e. after loading of aCD71-SPRN, a comparable high potency is observed (FIG. 6E, 6F, 6G). Even more so, when the aCD71-SPRN loading is first followed by a 6 hrs resting period, and then the saponin component is added, the potency is again very comparable to the co-administration FIG. 6E, 6F, 6G). Interestingly, and even more surprisingly, when a 24 hrs resting period is introduced between the end of toxin loading and the start of incubation of the cancer cells with saponin components, the efficacy enhancement of the aCD71-SPRN protein toxin is still apparent under influence of saponin component 1, 2 and 3 (FIG. 6E, 6F, 6G). Compared to the control treatment, there is still efficacy (i.e., cell killing) observable. This data shows that the efficacy of a proteinaceous effector moiety, here saporin in the context of an antibody-drug conjugate (ADC) can benefit from depot release stimulation, here by exposure to saponin components.

When the A2058, metastatic melanoma cells, were treated according to the same dosing scheme with a fixed concentration of aCD71-SPRN and a concentration range of different saponin components (according to dosing schemes in FIG. 6A, 6B, 6C, 6D), similar results were obtained as for the A431 cells. Also in the A2058 cells a very similar aCD71-SPRN potency enhancement by untargeted saponin components was observed between 48 hr co-administration and a treatment schedule in which the saponin component was added directly upon removal of the aCD71-SPRN protein toxin or after a 6 hr resting period (FIG. 7A, 7B); this sequential treatment regimen resulted in highly efficacious cell killing, which was much improved over cell killing without saponin components. Again, and highly surprisingly, even with the 24 hr resting period the efficacy enhancement of aCD71-SPRN was still apparent, and more potent than the control treatment without saponin components.

This suggests that epidermoid carcinoma cells and metastatic melanoma cells do have a depot for proteinaceous payloads and saponin components can effectively release the protein payload from these compartments. The dosing regimen might differ for a given payload, and for protein payloads a shorter resting period may be more beneficial to achieve full efficacy.

### Example 8: Depot Release by Saponin Components In Vivo: Tolerability, Efficacy and Durability of Effect of GN3-siRNA Under Influence of (Co-)Administration of Saponin Components in the Non-Human Primate

### NHP In Vivo Study Design (GN3-siAT3+GN3-Saponin)

Non-human primate (NHP) care and experimental procedures were performed in compliance with Council Directive No. 2010/63/EU and French decree No. 2013-118. Before study initiation, NHPs were acclimated in-house for two weeks, and a complete clinical examination and full clinical chemistry screening was performed. Details of the study design are shown in Table A5. In brief, 8 male non-human primates (Macaca fascicularis) were allocated to 5 dosing groups and received a single s.c. dose administration (1 ml/kg) of GN3-siAT3 (0.3 mg/kg) on day 1 (dosing day), which was for most groups combined with a single s.c. dose administration (1 ml/kg) of GN3-saponin on either day 1 (3, 1 or 0.3 mg/kg), or day 28 (0.1 mg/kg), see Table A5. Blood sampling (3 ml), collected via venipuncture, was performed before study initiation, and on day 3, 7, 14, 16, 21, 23, 28, 30, 35, 38, 42 and 45 (end of study), unless indicated otherwise in Table A5. After blood sampling, serum was prepared for antithrombin III (AT3) protein and clinical chemistry analysis. NHPs were terminated on day 45, unless indicated otherwise in Table A5. As a tolerability control group, one female NHP received a single s.c. dose administration of 3 mg/kg GN3-saponin on day 0, and blood was collected at several timepoints for extensive clinical pathology analysis (Table A5). Following sacrifice, liver samples were harvested for SERPINC1 mRNA analysis. Liver samples were preserved in RNAlater for 24-72 hours at 4° C., then snap frozen, and subsequently stored at −80° C. until analysis.

### Serum Preparation

Blood samples were collected in plain tubes with clot activator, allowed to clot for at least 30 minutes at room temperature, then centrifuged at 3,000×g at 400 for 10 minutes. Serum samples were aliquoted, frozen over dry ice or at −80° C. until analysis.

### Bioanalysis and Biochemistry Analysis

ALT and creatinine protein levels, as a reporter for liver and kidney tolerability, were assessed in serum samples with an Advia 1800 analyzer. AT3 protein levels were measured by ELISA using the Human AT3 AssayMax™ ELISA Kit (#EA3301-1, AssayPro LLC), according to the manufacturer's instructions.

TABLE A5
Dosing groups in vivo tolerability, efficacy and durability of different
dosing regimens of GN3-saponins (GN3-AH-SO1861) in combination with
trivalent-GalNAc-siRNA (GN3-siAT3) in non-human primates (NHP).

| Treatment, | | Dose, | Treatment | | | |
|---|---|---|---|---|---|---|
| Component | Treatment, | Component | Component | Number | Blood | Sacrifice |
| 1 | Component | 2 | 2 | of | sampling | [study |
| 0.3 mg/kg | 2 | [mg/kg] | [day] | animals | Timepoint | day] |
| GN3-siAT3 | n/a | 0 | n/a | 1 | predose, d 3, | d 45 |
| | | | | | d 7, d 14, d 16, | |
| | | | | | d 21, d 23, d 28, | |
| | | | | | d 30, d 35, d 38, | |
| | | | | | d 42, d 45 | |
| GN3-siAT3 | GN3- | 0.1 | 28 | 1 | predose, d 3, | d 45 |
| | SO1861 | | | | d 7, d 14, d 16, | |
| | | | | | d 21, d 23, d 28, | |
| | | | | | d 30, d 35, d 38, | |
| | | | | | d 42, d 45 | |
| GN3-siAT3 | GN3- | 0.3 | 1 | 2 | predose, d 3, | d 11¹, d 17¹ |
| | SO1861 | | (Co-Admin) | | d 7, (d 11)¹ or | |
| | | | | | d 14, (d 16, | |
| | | | | | d 17) | |
| GN3-siAT3 | GN3- | 1 | 1 | 2 | predose, d 3, | d 12¹, d 13¹ |
| | SO1861 | | (Co-Admin) | | d 7, d 12 or d 13 | |
| GN3-siAT3 | GN3- | 3 | 1 | 2 | predose, d 3, | d 7¹, d 11¹ |
| | SO1861 | | (Co-Admin) | | d 7, (d 11)¹ | |
| n/a | GN3- | 3 | n/a | 1 | predose, d 3, | d 15 |
| | SO1861 | | | | d 9, d 12, d 14, | |
| | | | | | d 15 | |

¹NHPs reached the humane endpoint (related to exaggerated efficacy) and were prematurely terminated.

### Results

GN3-saponin efficacy translation to higher species was investigated in non-human primates (NHPs) using GN3-saponin and an GN3-siRNA targeting the antithrombin III (AT3)-encoding SERPINC1 mRNA (GN3-siAT3). A compound with the same sequence and chemical modifications as GN3-siAT3 is currently being evaluated in Phase 3 clinical studies under the INN fitusiran, as a therapeutic suppressing AT3 protein to promote hemostasis in severe hemophilia patients. Fitusiran is active with a well described dose and PD half-life (Boianelli, A., Aoki, Y., Ivanov, M., Dahlen, A. and Gennemark, P. (2022) Cross-Species Translation of Biophase Half-Life and Potency of GalNAc-Conjugated siRNAs. Nucleic Acid Ther, 32, 507-512 (Boianelli et al.)), and good interspecies PK/PD model (Boianelli et al.). A single dose of 30 mg/kg of a GN3-siRNA targeting SERPINC1 was shown to result in >90% AT3 protein reduction in NHPs (Sehgal, A., Barros, S., Ivanciu, L., Cooley, B., Qin, J., Racie, T., Hettinger, J., Carioto, M., Jiang, Y., Brodsky, J., et al. (2015) An RNAi therapeutic targeting antithrombin to rebalance the coagulation system and promote hemostasis in hemophilia. Nat Med, 21, 492-497). The targeted therapeutic range of AT3 in hemophilia patients is 15-35% remaining AT3 protein (Young, G., Lenting, P. J., Croteau, S. E., Nolan, B. and Srivastava, A. (2023) Antithrombin lowering in hemophilia: a closer look at fitusiran. Res Pract Thromb Haemost, 7 (Young et al.); Kaddi C et al. (2022) Development of a Quantitative Systems Pharmacology Model to Explore Hemostatic Equivalency of Antithrombin Lowering. Blood (2022) 140 (Supplement 1): 5606-5607. https://doi.org/10.1182/blood-2022-169043 [conference abstract]). Low AT3 levels have been shown to increase the thrombotic risk (Young et al.).

To test whether GN3-saponin can improve the efficacy of GN3-siRNA (here GN3-siAT3), NHPs received a suboptimal dose of 0.3 mg/kg GN3-siRNA on day 1. This suboptimal dose should allow for (at least) a low level of efficacy of GN3-siRNA on its own (as extrapolated from (Boianelli et al.), while leaving a 3-5-fold window for efficacy improvement by GN3-saponin before leading to AT3 protein levels below 10% in blood, associated with increased thrombosis risk and incidence (Young et al.). As expected, treatment with 0.3 mg/kg GN3-siRNA alone resulted in low-level (maximally up to 27%) reduction in AT3 protein levels (FIG. 12). In contrast, co-administration of 0.3 mg/kg GN3-siRNA with 0.3, 1 or 3 mg/kg GN3-saponin on day 1 resulted in a fast and strong reduction in AT3 protein levels, achieving around 90% or more AT3 serum reduction within the first 7 days of treatment (FIG. 12A, 12B, 12C). Interestingly, the 3 different co-administered doses of GN3-SPT (to 0.3 mg/kg GN3-siRNA) on day 1, all showed comparable results (FIG. 12A, 12B, 12C), indicating that the minimal efficacious dose of GN3-saponin to induce maximal siRNA release from endosomal compartments is 0.3 mg/kg or lower. Note that within two weeks of co-administration, AT3 protein levels dropped below a critical AT3 level of <10%. The low AT3 levels were associated with several concomitant clinical and clinical chemistry findings, including a marked increase in prothrombin time (PT) and activated partial thromboplastin time (aPTT) and other signs of a thrombotic event, ultimately leading to NHPs meeting their humane endpoint. Accordingly, analysis of hepatic SERPINC1 mRNA levels at termination revealed almost complete SERPINC1 knockdown (<2% remaining) after co-administration of either 1 or 0.3 mg/kg GN3-saponion with GN3-siRNA, which is a striking reduction in SERPINC1 levels compared to treatment with GN3-siRNA alone. Together, the data support the interpretation that upon treatment with GN3-siRNA alone, the bulk of GN3-siRNA remains trapped in endosomes and insufficiently reaches its intracellular target. Co-treatment with only 0.3 mg/kg GN3-SPT already markedly enhances endosomal GN3-siRNA release and thus potency of GN3-siRNA, resulting in a strong, and even exaggerated PD effect, of a suboptimal dose of GN3-siRNA. One NHP was treated with a supra-efficacious dose of 3 mg/kg GN3-saponin alone. This dose was well tolerated without any clinical findings, without changes in an extensive clinical pathology panel (clinical chemistry, including ALT levels, hematology, and coagulation), nor findings in gross organ morphology or renal and kidney histopathology (data not shown).

To further assess whether GN3-siRNA was indeed trapped in endosomes and to test the minimally efficacious GN3-saponin dose, 0.1 mg/kg GN3-saponin was administered at a delayed timepoint (day 28) to 0.3 mg/kg GN3-siRNA (day 1) (FIG. 13). While treatment with GN3-siAT3 (or GN3-siRNA) alone did not meaningfully reduce AT3 protein levels, strong AT3 protein knockdown (78%) was achieved after delayed boost administration with GN3-saponin, and the effect was maintained for at least 17 days through the study end at day 45. Interestingly, this treatment realized a clinically meaningful AT3 reduction, to a residual AT3 level in the range of 15-35%, while the GN3-siRNA dose used was not efficacious without co-treatment with GN3-saponin. Accordingly, a strong reduction in hepatic SERPINC1 levels was observed (35% remaining) compared to treatment with GN3-siRNA alone, as observed in a PCR analysis. Taken together, these data support the interpretation of initial delivery and entrapment of GN3-siRNA in endosomal compartments, and the ability of low-dose GN3-saponin to reach such depot endosomes and release entrapped GN3-siRNA, allowing it to reach its intracellular target, even 4 weeks after initial GN3-siRNA dosing.

In summary, these data show that co-treatment with GN3-saponin was not only able to enhance the efficacy of GN3-siRNA, but also enables a delayed boost with good durability of effect when administered at a delayed timepoint to GN3-siRNA. Interestingly, the results also indicate that the minimal efficacious dose of GN3-saponin is 0.1 mg/kg or lower in NHPs, when applied 28 days after GN3-siRNA treatment. Further, co-treatment with GN3-saponin apparently enables a ˜100-fold dose reduction of GN3-siRNA compared to previous studies (Sehgal, A. et al.). This finding is even more remarkable when taking the half-life of GN3-siAT3 into account. The non-empirical, PD-based prediction of half-life of GN3-siAT3 is ˜6 days in NHPs, as modelled by Boianelli et al, which would mean that at day 28, i.e. after ˜4-5 half-lives, only <5% intact GN3-siAT3 remains. This would equal an effective dose of less than 0.02 mg/kg GN3-siAT3 available, which was efficiently released from endosomal compartments by low-dose GN3-saponin. Together, these findings show successful concept translation from mice to higher species and indicate that the minimal efficacious dose is 0.1 mg/kg GN3-saponin or lower in NHPs.

1.-36. (canceled)

37. A therapeutic method of treating a subject suffering from a disease or condition related to a defect in the expression of a gene and/or a disease or condition that is treatable by modulating the expression and/or expression level of a gene; said therapeutic method comprising:
i) the administration, to said subject, preferably the repeated administration, of a nucleic acid or oligonucleotide therapeutic that is capable of modulating the expression of said gene and/or modulating the expression level of said gene; and
ii) the administration, to said subject, of the saponin component;
wherein the saponin component is administered after the nucleic acid or oligonucleotide therapeutic has been administered, resulting in an extension of the duration of effect of the nucleic acid or oligonucleotide therapeutic and/or in an extension of the dosing interval of the nucleic acid or oligonucleotide therapeutic and/or in a reduction of the dosing frequency of the nucleic acid or oligonucleotide therapeutic and/or in a (delayed) boost in the effect of the nucleic acid or oligonucleotide therapeutic.

38. The therapeutic method according to claim 37, wherein said saponin component comprises a saponin moiety or consists of a saponin molecule, wherein the saponin moiety or the saponin molecule is a penta-cyclic triterpene saponin of the 12,13-dehydrooleanane type; preferably a penta-cyclic triterpene saponin of the 12,13-dehydrooleanane type comprising an aldehyde function in position C-23 of the aglycone core structure of the saponin molecule; and/or wherein the saponin moiety or the saponin molecule is a mono-desmosidic saponin or a bi-desmosidic saponin, preferably a bi-desmosidic saponin; and/or
wherein the saponin moiety or the saponin molecule comprises a first saccharide chain bound to position C-3 of its aglycone core structure, selected from Group A listed in Table 1A, wherein preferably said first saccharide chain of the saponin molecule comprises a glucuronic acid group or optionally said first saccharide chain of the saponin moiety comprises a glucuronic acid group, preferably the saponin moiety or the saponin molecule comprises a first saccharide chain Gal-(1→2)-[Xyl-(1→3)]-GlcA; and/or
wherein the saponin moiety or the saponin molecule comprises an aglycone core structure selected from quillaic acid, gypsogenin, and a derivative thereof.

39. The therapeutic method according to claim 37, wherein said saponin component comprises a saponin moiety or consists of a saponin molecule, wherein the saponin moiety or the saponin molecule comprises:
a gypsogenin aglycone core structure and is selected from:
SA1641, gypsoside A, NP-017772, NP-017774, NP-017777, NP-017778, NP-018109, NP-017888, NP-017889, NP-018108, S01658 and Phytolaccagenin; or
a quillaic acid aglycone core structure and is selected from:
AG1856, AG1, AG2, Agrostemmoside E, GE1741, Gypsophila saponin 1 (Gyp1), NP-017674, NP-017810, NP-003881, NP-017676, NP-017677, NP-017705, NP-017706, NP-017773, NP-017775, SA1657, Saponarioside B, S01542, S01584, S01674, S01700, S01730, S01772, S01832, SO1861, SO1862, S01904, QS-7, QS-7 api, QS-17, QS-18, QS-21 A-apio, QS-21 A-xylo, QS-21 B-apio and QS-21 B-xylo.

40. The therapeutic method according to claim 37, wherein said saponin component comprises a saponin moiety with covalently bound thereto a linker, or wherein said saponin component comprises a saponin moiety with covalently bound thereto a linker with a further molecule covalently bound to the linker, wherein said further molecule is a ligand for binding to an endocytic cell-receptor.

41. The therapeutic method according to claim 40, wherein the ligand is a proteinaceous ligand or a non-proteinaceous ligand or a combination thereof.

42. The therapeutic method according to claim 40, wherein the ligand is a protein ligand capable of binding to an endocytic cell-surface receptor, such as a cytokine or EGF; an antibody; a non-proteinaceous ligand capable of binding to an endocytic cell-surface receptor, such as a ligand for asialoglycoprotein receptor (ASGPR), preferably one or more GalNAc moieties, more preferably three GalNAc moieties.

43. The therapeutic method according to claim 37, wherein the nucleic acid or oligonucleotide therapeutic is selected from the group consisting of deoxyribonucleic acid (DNA) oligomer, ribonucleic acid (RNA) oligomer, antisense oligonucleotide (ASO, AON), short interfering RNA (siRNA), anti-microRNA (anti-miRNA), DNA aptamer, RNA aptamer, mRNA, mini-circle DNA, peptide nucleic acid (PNA), phosphoramidate morpholino oligomer (PMO), phosphorothioate-modified antisense oligonucleotide (PS-ASO), 2′-O-methyl (2′-OMe) phosphorothioate RNA, 2′-O-methoxyethyl (2′-O-MOE) RNA {2′-O-methoxyethyl-RNA (MOE)}, locked nucleic acid (LNA), bridged nucleic acid (BNA), 2′-deoxy-2′-fluoroarabino nucleic acid (FANA), 2′-O-methoxyethyl-RNA (MOE), 3′-fluoro hexitol nucleic acid (FHNA), glycol nucleic acid (GNA), xeno nucleic acid oligonucleotide and threose nucleic acid (TNA); and/or
is selected from the group consisting of short interfering RNA (siRNA), short hairpin RNA (shRNA), anti-hairpin-shaped microRNA (miRNA), single-stranded RNA, aptamer RNA, double-stranded RNA (dsRNA), anti-microRNA (anti-miRNA, anti-miR), antisense oligonucleotide (ASO), mRNA, DNA, antisense DNA, locked nucleic acid (LNA), bridged nucleic acid (BNA), 2′-O,4′-aminoethylene bridged nucleic Acid (BNANC), BNA-based siRNA, and BNA-based antisense oligonucleotide (BNA-AON).

44. The therapeutic method according to claim 37, wherein the nucleic acid or oligonucleotide therapeutic is capable of silencing a gene, when present in a cell comprising such gene, preferably wherein the gene is any one of the following genes: apolipoprotein B (apoB), HSP27, transthyretin (TTR), proprotein convertase subtilisin/kexin type 9 (PCSK9), delta-aminolevulinate synthase 1 (ALAS1), antithrombin 3 (AT3), glycolate oxidase (GO), complement component C5 (CC5), X gene of hepatitis B virus (HBV), S gene of HBV, alpha-1 antitrypsin (AAT) and lactate dehydrogenase (LDH), and/or is capable of targeting an aberrant miRNA when present in a cell comprising such aberrant miRNA; and/or
wherein the nucleic acid or oligonucleotide therapeutic targets a gene selected from the group consisting of: IRS1, ICAM1, TTR, FUS, APOC3, LPA, CEP290, SOD1, HTT, TGFB2, GFAP, CCR3/CSF2RB, GHR, ITGA4, PCSK9, FOXP3, viral HBV, viral UL123, ApoB100, ARSA, ALAS, GO, VEGF, MAPT, PCED, STAT3, KLB1, DYN2, UBE2A, DGAT2, SNCA, ATXN2, LRRK2, AGT, F11, GCGR, KLBK1, AR, SCNN1A, TMPRSS6, TGFB2, DMD (dystrophin), GRB2, RHO, USH2A, SCN1A, ANGPTL3, C2orf72, SERPINC1, LDHA, CASP2, TP53, TRPV1, SERPINA1, HSD17B13, ANGPTL3, APOC3, ADRB2, SERPINA1, SERPINHI, C5, CHST15, CTGF, KRAS, PTGS2/TGFB1, HBsAG, MIR21, CEBPA, MIR29B1, ALAS1, HAO1, SMN2, APOB, CMV virus IE2, GJA1 and CFB.

45. The therapeutic method according to claim 37, wherein the disease or condition is selected from the group consisting of: Homozygous familial hypercholesterolemia and/or (other) apoB-100-related diseases; Spinal muscular atrophy and/or (other) SMN2-related diseases; Duchenne muscular dystrophy and/or (other) DMD-related diseases; Veno-occlusive disease and/or (other) ARSA-related diseases; Primary hypercholesterolemia and/or (other) PCSK9-related diseases; Familial chylomicronemia syndrome; and/or (other) apoC3-related diseases; Duchenne muscular dystrophy (DMD), and/or (other) ITGA4-related diseases; chronic hepatitis B (CHB), and/or (other) viral HBV-related diseases; hypercholesterolaemia, and/or (other) PCSK9-related diseases; congenital structural myopathies, and/or (other) DYN2-related diseases; non-alcoholic steatohepatitis (NASH), and/or (other) DGAT2-related diseases; complement-mediated diseases, including IgA nephropathy and Age-related macular degeneration (AMD); clotting disorders, including thrombosis, and end-stage renal disease (ESRD), and/or (other) F11-related diseases; hepatitis B virus (HBV) infections, and/or (other) viral HBV-related diseases; hepatic steatosis, and hypertriglyceridaemia, and/or (other) ANGPTL3-related diseases; primary hyperoxaluria (PH), and/or (other) LDHA-related diseases; hepatitis B virus (HBV) infections, and/or (other) HBsAg-related diseases; gastrointestinal disorders including non-alcoholic steatohepatitis, and/or (other) HSD17B13-related diseases; homozygous familial hypercholesterolemia, and/or (other) ANGPTL3-related diseases; immunoglobulin A nephropathy, and/or (other) C5-related diseases; alpha-1 antitrypsin (AAT) deficiency-associated liver disease (AATLD), and/or (other) SERPINA1-related diseases; hepatitis B virus (HBV) infections, and/or (other) viral HBV-related diseases; chronic hepatitis B virus (HBV) infection, and/or (other) HBsAg-related diseases; basal cell cancer, Bowen's disease, hypertrophic scars, keloids, Cholangiocarcinoma, Liver cancer, obesity, bladder cancer, and/or (other) PTGS2- or TGFB1-related diseases; hepatitis B and hepatitis D virus infections, and/or (other) HBsAg-related diseases; Alport Syndrome, and/or (other) MIR21-related diseases; liver cancer, and/or (other) CEBPA-related diseases.

46. The therapeutic method according to claim 37, wherein the nucleic acid or oligonucleotide therapeutic is a nucleic acid or oligonucleotide therapeutic with covalently bound thereto a linker with a further molecule covalently bound to the linker, wherein said further molecule is a ligand for binding to an endocytic cell-surface receptor.

47. The therapeutic method according to claim 46, wherein the ligand is a proteinaceous ligand or a non-proteinaceous ligand or a combination thereof.

48. The therapeutic method according to claim 46, wherein the ligand is a protein ligand capable of binding to an endocytic cell-surface receptor, such as a cytokine or EGF; an antibody; a non-proteinaceous ligand capable of binding to an endocytic cell-surface receptor, such as a ligand for asialoglycoprotein receptor (ASGPR), preferably one or more GalNAc moieties, more preferably three GalNAc moieties.

49. The therapeutic method according to claim 37, wherein the nucleic acid or oligonucleotide therapeutic and the saponin component both comprise a ligand, wherein the nucleic acid or oligonucleotide therapeutic either comprises the same ligand as the saponin component, or the nucleic acid or oligonucleotide therapeutic comprises a ligand that differs from the ligand comprised by the saponin component, with the proviso that the different ligands both bind to an endocytic cell-surface receptor present on the same cell.

50. The therapeutic method according to claim 37, wherein the saponin component is administered after the moment the peak in pharmacological effect following the administration of the effector component has been reached, preferably the saponin component is administered at least 1 day after the nucleic acid or oligonucleotide therapeutic is administered, preferably at least 2 days, at least 3 days, at least 1 week, at least 2 weeks, at least 3 weeks, at least 1 month, at least 2 months, at least 3 months, at least 4 months, at least 5 months, or at least 6 months after the nucleic acid or oligonucleotide therapeutic is administered.

51. The therapeutic method according to claim 37, wherein the saponin component is administered during the second, third or fourth quarter of the normal dosing interval of the nucleic acid or oligonucleotide therapeutic; and/or
wherein the saponin component is administered during the second half of the normal dosing interval of the nucleic acid or oligonucleotide therapeutic.

52. The therapeutic method according to claim 37, wherein the nucleic acid or oligonucleotide therapeutic is administered during the fourth quarter of the normal dosing interval of the nucleic acid or oligonucleotide therapeutic.

53. The therapeutic method according to claim 37, wherein said therapeutic method results in the prolonging of the effect of the nucleic acid or oligonucleotide therapeutic and/or the prolonging of the dosing interval of the nucleic acid or oligonucleotide therapeutic by a factor 1.25 or more, preferably a factor 1.5, more preferably a factor 2; and/or
results in the reduction of the dosing frequency of the nucleic acid or oligonucleotide therapeutic by a factor 0.90 or lower, preferably a factor 0.75, more preferably a factor 0.5.

54. The therapeutic method according to claim 37, wherein during an administration interval of the nucleic acid or oligonucleotide therapeutic, the saponin component is administered at least once, such as once, twice, three times or four times, preferably at fixed/regular intervals; and/or
wherein during each administration interval of the nucleic acid or oligonucleotide therapeutic, the saponin component is administered at least once, such as once, twice, three times or four times, preferably at fixed/regular intervals.

55. A therapeutic method of treating a subject suffering from a disease or condition related to a defect in the expression of a gene and/or a disease or condition that is treatable by modulating the expression and/or expression level of said gene, said therapeutic method comprising administration of a pharmaceutical combination comprising a saponin component and a nucleic acid or oligonucleotide therapeutic that is capable of regulating the expression of a gene and/or that is capable of modulating the expression level of said gene, wherein
i) the administration, to said subject, preferably the repeated administration, of said nucleic acid or oligonucleotide therapeutic; and
ii) the administration, to said subject, of the saponin component;
wherein the saponin component is administered after the nucleic acid or oligonucleotide therapeutic has been administered, resulting in an extension of the duration of effect of the nucleic acid or oligonucleotide therapeutic and/or in an extension of the dosing interval of the nucleic acid or oligonucleotide therapeutic and/or in a reduction of the dosing frequency of the nucleic acid or oligonucleotide therapeutic and/or in a (delayed) boost in the effect of the nucleic acid or oligonucleotide therapeutic.

56. A pharmaceutical kit comprising a package comprising a) one or more dosage units comprising a saponin component, and b) printed instructions to use the dosage units comprised in the kit in a therapeutic method of treatment of a disease or condition related to a defect in the expression of a gene and/or a disease or condition that is treatable by modulating the expression and/or expression level of a gene, said method of treatment comprising: [symbol]
